SEQUENCE LISTING (353 sequences)

SEQ ID NO: 1 - DNA, 363 bp, synthetic construct
  misc_feature 1..363: note = Synthetic
  source 1..363: mol_type = other DNA
gaggtgcagctggtggagtctgggggaggcttggtccagcctggggggtccctgagactctcctgtgcagcctctggattcacctttagtaggttttggatgagctgggtccgccaggctccagggaaggggctggagtgggtggccaacataaaccaagatggaactgagaaatactatgtggactctgtgaagggccgattcaccatctccagagacaacgccaagaactcactgtatctgcaaatgaacagcctgagagccggggacacggctgtgtattactgtgcgaatacgtattacgatttttggagtggtcactttgactactggggccagggaaccctggtcaccgtctcctca

SEQ ID NO: 2 - protein, 121 aa, synthetic construct
  REGION 1..121: note = Synthetic
EVQLVESGGGLVQPGGSLRLSCAASGFTFSRFWMSWVRQAPGKGLEWVANINQDGTEKYYVDSVKGRFTISRDNAKNSLYLQMNSLRAGDTAVYYCANTYYDFWSGHFDYWGQGTLVTVSS

SEQ ID NO: 3 - DNA, 24 bp, synthetic construct
  misc_feature 1..24: note = Synthetic
  source 1..24: mol_type = other DNA
ggattcacctttagtaggttttgg

SEQ ID NO: 4 - protein, 8 aa, synthetic construct
  REGION 1..8: note = Synthetic
GFTFSRFW

SEQ ID NO: 5 - DNA, 24 bp, synthetic construct
  misc_feature 1..24: note = Synthetic
  source 1..24: mol_type = other DNA
ataaaccaagatggaactgagaaa

SEQ ID NO: 6 - protein, 8 aa, synthetic construct
  REGION 1..8: note = Synthetic
INQDGTEK

SEQ ID NO: 7 - DNA, 42 bp, synthetic construct
  misc_feature 1..42: note = Synthetic
  source 1..42: mol_type = other DNA
gcgaatacgtattacgatttttggagtggtcactttgactac

SEQ ID NO: 8 - protein, 14 aa, synthetic construct
  REGION 1..14: note = Synthetic
ANTYYDFWSGHFDY

SEQ ID NO: 9 - DNA, 321 bp, synthetic construct
  misc_feature 1..321: note = Synthetic
  source 1..321: mol_type = other DNA
gacatccagatgacccagtctccttccaccctgtctgcatctgtaggagacagagtcaccatcacttgtcgggccagtcagagtattagtaattggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtatcatagttattcgtacacttttggccaggggaccaagctggagatcaaa

SEQ ID NO: 10 - protein, 107 aa, synthetic construct
  REGION 1..107: note = Synthetic
DIQMTQSPSTLSASVGDRVTITCRASQSISNWLAWYQQKPGKAPKLLIYKASSLESGVPSRFSGSGSGTEFTLTISSLQPDDFATYYCQQYHSYSYTFGQGTKLEIK

SEQ ID NO: 11 - DNA, 18 bp, synthetic construct
  misc_feature 1..18: note = Synthetic
  source 1..18: mol_type = other DNA
cagagtattagtaattgg

SEQ ID NO: 12 - protein, 6 aa, synthetic construct
  REGION 1..6: note = Synthetic
QSISNW

SEQ ID NO: 13-14 - skipped (2 entries)

SEQ ID NO: 15 - DNA, 27 bp, synthetic construct
  misc_feature 1..27: note = Synthetic
  source 1..27: mol_type = other DNA
caacagtatcatagttattcgtacact

SEQ ID NO: 16 - protein, 9 aa, synthetic construct
  REGION 1..9: note = Synthetic
QQYHSYSYT

SEQ ID NO: 17 - DNA, 363 bp, synthetic construct
  misc_feature 1..363: note = Synthetic
  source 1..363: mol_type = other DNA
caggagcacctggtggagtctgggggaggcgtggtccagcctgggaggtccctgagactctcctgtgaagcgtctggattcaccttcagtaactttggcatgcactgggtccgccaggctccaggcaaggggctggagtgggtggcagctttatggtctgatggaagtaataaatactatgcagactccgtgaagggtcgagtcaccatctccagagacaattccaagaacacactgtatctgcaaatgaacagcctgagagccgaggacacggctgtctattactgtgcgagagggagaggagcccccggtattccgatttttgggtactggggccagggaaccctggtcaccgtctcctca

SEQ ID NO: 18 - protein, 121 aa, synthetic construct
  REGION 1..121: note = Synthetic
QEHLVESGGGVVQPGRSLRLSCEASGFTFSNFGMHWVRQAPGKGLEWVAALWSDGSNKYYADSVKGRVTISRDNSKNTLYLQMNSLRAEDTAVYYCARGRGAPGIPIFGYWGQGTLVTVSS

SEQ ID NO: 19 - DNA, 24 bp, synthetic construct
  misc_feature 1..24: note = Synthetic
  source 1..24: mol_type = other DNA
ggattcaccttcagtaactttggc

SEQ ID NO: 20 - protein, 8 aa, synthetic construct
  REGION 1..8: note = Synthetic
GFTFSNFG

SEQ ID NO: 21 - DNA, 24 bp, synthetic construct
  misc_feature 1..24: note = Synthetic
  source 1..24: mol_type = other DNA
ttatggtctgatggaagtaataaa

SEQ ID NO: 22 - protein, 8 aa, synthetic construct
  REGION 1..8: note = Synthetic
LWSDGSNK

SEQ ID NO: 23 - DNA, 42 bp, synthetic construct
  misc_feature 1..42: note = Synthetic
  source 1..42: mol_type = other DNA
gcgagagggagaggagcccccggtattccgatttttgggtac

SEQ ID NO: 24 - protein, 14 aa, synthetic construct
  REGION 1..14: note = Synthetic
ARGRGAPGIPIFGY

SEQ ID NO: 25 - DNA, 321 bp, synthetic construct
  misc_feature 1..321: note = Synthetic
  source 1..321: mol_type = other DNA
gacatccagatgacccagtctccatcctccctgtctgcatctgttggagacagagtcaccatcacttgccgggcaagtcagggcattagaaatgatttaggctggtatcagcagaaaccagggaaagcccctaagcgcctgatctatactgcatccagtttgcaaagtggggtcccatcaagattcagcggcagtggatctgggacagaattcactctcacaatcagcagcctacagcctgaagattttgcaacttattactgtctacaacataatagttaccctctcacattcggcggagggaccaaggtggcgatcaaa

SEQ ID NO: 26 - protein, 107 aa, synthetic construct
  REGION 1..107: note = Synthetic
DIQMTQSPSSLSASVGDRVTITCRASQGIRNDLGWYQQKPGKAPKRLIYTASSLQSGVPSRFSGSGSGTEFTLTISSLQPEDFATYYCLQHNSYPLTFGGGTKVAIK

SEQ ID NO: 27 - DNA, 18 bp, synthetic construct
  misc_feature 1..18: note = Synthetic
  source 1..18: mol_type = other DNA
cagggcattagaaatgat

SEQ ID NO: 28 - protein, 6 aa, synthetic construct
  REGION 1..6: note = Synthetic
QGIRND

SEQ ID NO: 29-30 - skipped (2 entries)

SEQ ID NO: 31 - DNA, 27 bp, synthetic construct
  misc_feature 1..27: note = Synthetic
  source 1..27: mol_type = other DNA
ctacaacataatagttaccctctcaca

SEQ ID NO: 32 - protein, 9 aa, synthetic construct
  REGION 1..9: note = Synthetic
LQHNSYPLT

SEQ ID NO: 33 - DNA, 390 bp, synthetic construct
  misc_feature 1..390: note = Synthetic
  source 1..390: mol_type = other DNA
gaggtgcagctggtggagtctgggggaggcttggtaaagcctggggggtcccttagactctcctgtgcagcctctggattcactttcagtaacgcctggatgagctgggtccgccaggctccagggaaggggctggagtgggttggccgtattaaaaggaaaactgatggtgggacaacagactacgctgcacccgtgaaaggcagattcaccatctcaagagatgattcaaaaaatacgctgcatctgcaaatgaacagcctgaaaaccgaggacacagccgtgtattactgtaccacagatgatattgtagttgtaccagctgttatgagggaatactacttcggtatggacgtctggggccaagggaccacggtcaccgtctcctca

SEQ ID NO: 34 - protein, 130 aa, synthetic construct
  REGION 1..130: note = Synthetic
EVQLVESGGGLVKPGGSLRLSCAASGFTFSNAWMSWVRQAPGKGLEWVGRIKRKTDGGTTDYAAPVKGRFTISRDDSKNTLHLQMNSLKTEDTAVYYCTTDDIVVVPAVMREYYFGMDVWGQGTTVTVSS

SEQ ID NO: 35 - DNA, 24 bp, synthetic construct
  misc_feature 1..24: note = Synthetic
  source 1..24: mol_type = other DNA
ggattcactttcagtaacgcctgg

SEQ ID NO: 36 - protein, 8 aa, synthetic construct
  REGION 1..8: note = Synthetic
GFTFSNAW

SEQ ID NO: 37 - DNA, 30 bp, synthetic construct
  misc_feature 1..30: note = Synthetic
  source 1..30: mol_type = other DNA
attaaaaggaaaactgatggtgggacaaca

SEQ ID NO: 38 - protein, 10 aa, synthetic construct
  REGION 1..10: note = Synthetic
IKRKTDGGTT

SEQ ID NO: 39 - DNA, 63 bp, synthetic construct
  misc_feature 1..63: note = Synthetic
  source 1..63: mol_type = other DNA
accacagatgatattgtagttgtaccagctgttatgagggaatactacttcggtatggacgtc

SEQ ID NO: 40 - protein, 21 aa, synthetic construct
  REGION 1..21: note = Synthetic
TTDDIVVVPAVMREYYFGMDV

SEQ ID NO: 41 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccggacaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataataattacccgtacacttttggccaggggaccaagctggagatcaaa

SEQ ID NO: 42 - protein, 107 aa, synthetic construct
  REGION 1..107: note = Synthetic
DIQMTQSPSSLSASVGDRVTITCRTSQGIRNDLGWYQQKPGKAPKRLIYAASSLQSGVPSRFSGSGSGTEFTLTISSLQPEDFATYYCLQHNNYPYTFGQGTKLEIK

SEQ ID NO: 43 - DNA, 18 bp, synthetic construct
  misc_feature 1..18: note = Synthetic
  source 1..18: mol_type = other DNA
cagggcattagaaatgat

SEQ ID NO: 44 - protein, 6 aa, synthetic construct
  REGION 1..6: note = Synthetic
QGIRND

SEQ ID NO: 45-46 - skipped (2 entries)

SEQ ID NO: 47 - DNA, 27 bp, synthetic construct
  misc_feature 1..27: note = Synthetic
  source 1..27: mol_type = other DNA
ctacagcataataattacccgtacact

SEQ ID NO: 48 - protein, 9 aa, synthetic construct
  REGION 1..9: note = Synthetic
LQHNNYPYT

SEQ ID NO: 49 - DNA, 363 bp, synthetic construct
  misc_feature 1..363: note = Synthetic
  source 1..363: mol_type = other DNA
caggtgcaattggtgcagtctggggcggaggtgaagaagcctggggcctcagtgcaggtctcctgcaaggcttctggatactccttcaccggctactatatacactgggtgcgacaggcccctggacaaggacttgagtggatgggatggatcaaccctaacagtggcaccaaaaagtatgcacacaagtttcagggcagggtcaccatgaccagggacacgtccatcgacacagcctacatgattttgagcagtctgatatccgacgacacggccgtgtattactgtgcgagagatgaggactggaactttgggagctggttcgactcctggggccagggaaccctggtcaccgtctcctca

SEQ ID NO: 50 - protein, 121 aa, synthetic construct
  REGION 1..121: note = Synthetic
QVQLVQSGAEVKKPGASVQVSCKASGYSFTGYYIHWVRQAPGQGLEWMGWINPNSGTKKYAHKFQGRVTMTRDTSIDTAYMILSSLISDDTAVYYCARDEDWNFGSWFDSWGQGTLVTVSS

SEQ ID NO: 51 - DNA, 24 bp, synthetic construct
  misc_feature 1..24: note = Synthetic
  source 1..24: mol_type = other DNA
ggatactccttcaccggctactat

SEQ ID NO: 52 - protein, 8 aa, synthetic construct
  REGION 1..8: note = Synthetic
GYSFTGYY

SEQ ID NO: 53 - DNA, 24 bp, synthetic construct
  misc_feature 1..24: note = Synthetic
  source 1..24: mol_type = other DNA
atcaaccctaacagtggcaccaaa

SEQ ID NO: 54 - protein, 8 aa, synthetic construct
  REGION 1..8: note = Synthetic
INPNSGTK

SEQ ID NO: 55 - DNA, 42 bp, synthetic construct
  misc_feature 1..42: note = Synthetic
  source 1..42: mol_type = other DNA
gcgagagatgaggactggaactttgggagctggttcgactcc

SEQ ID NO: 56 - protein, 14 aa, synthetic construct
  REGION 1..14: note = Synthetic
ARDEDWNFGSWFDS

SEQ ID NO: 57 - DNA, 336 bp, synthetic construct
  misc_feature 1..336: note = Synthetic
  source 1..336: mol_type = other DNA
gatattgtgatgacccagactccactctcctcacctgtcacccttggacagccggcctccatctcctgcaggtctagtcaaaccctcgtacacggtgatggaaacacgtacttgagttggattcagcagaggccaggccagcctccgagactcctcatttataaggtttctaatcagttctctggggtcccagacagattcagtggcagtggggcagggacagatttcacactgaaaatcagcagggtggaagctgaggatgtcgggctttatttctgcatgcaagctacacattttccgatcaccttcggccaagggacacgactggagattaaa

SEQ ID NO: 58 - protein, 112 aa, synthetic construct
  REGION 1..112: note = Synthetic
DIVMTQTPLSSPVTLGQPASISCRSSQTLVHGDGNTYLSWIQQRPGQPPRLLIYKVSNQFSGVPDRFSGSGAGTDFTLKISRVEAEDVGLYFCMQATHFPITFGQGTRLEIK

SEQ ID NO: 59 - DNA, 33 bp, synthetic construct
  misc_feature 1..33: note = Synthetic
  source 1..33: mol_type = other DNA
caaaccctcgtacacggtgatggaaacacgtac

SEQ ID NO: 60 - protein, 11 aa, synthetic construct
  REGION 1..11: note = Synthetic
QTLVHGDGNTY

SEQ ID NO: 61-62 - skipped (2 entries)

SEQ ID NO: 63 - DNA, 27 bp, synthetic construct
  misc_feature 1..27: note = Synthetic
  source 1..27: mol_type = other DNA
atgcaagctacacattttccgatcacc

SEQ ID NO: 64 - protein, 9 aa, synthetic construct
  REGION 1..9: note = Synthetic
MQATHFPIT

SEQ ID NO: 65 - DNA, 363 bp, synthetic construct
  misc_feature 1..363: note = Synthetic
  source 1..363: mol_type = other DNA
caggtacacctggtgcagtctggggctgaggtgaagaagcctggggcctcagtgaaggtctcctgcaaggcttctggatacaccttcaccggctactatatacattgggtgcgacaggcccctggacacgggcttgagtggatgggatggctcaaccctaatactggtaccacaaagtatatacagaactttcagggcagggtcaccatgaccagggacacgtccagcagcacagcctacatggagctgaccaggctgagatctgacgacacggccgtgtattactgtgcgagagatgaggactggaattatgggagctggttcgacacctggggccagggaaccctggtcacagtctcctca

SEQ ID NO: 66 - protein, 121 aa, synthetic construct
  REGION 1..121: note = Synthetic
QVHLVQSGAEVKKPGASVKVSCKASGYTFTGYYIHWVRQAPGHGLEWMGWLNPNTGTTKYIQNFQGRVTMTRDTSSSTAYMELTRLRSDDTAVYYCARDEDWNYGSWFDTWGQGTLVTVSS

SEQ ID NO: 67 - DNA, 24 bp, synthetic construct
  misc_feature 1..24: note = Synthetic
  source 1..24: mol_type = other DNA
ggatacaccttcaccggctactat

SEQ ID NO: 68 - protein, 8 aa, synthetic construct
  REGION 1..8: note = Synthetic
GYTFTGYY

SEQ ID NO: 69 - DNA, 24 bp, synthetic construct
  misc_feature 1..24: note = Synthetic
  source 1..24: mol_type = other DNA
ctcaaccctaatactggtaccaca

SEQ ID NO: 70 - protein, 8 aa, synthetic construct
  REGION 1..8: note = Synthetic
LNPNTGTT

SEQ ID NO: 71 - DNA, 42 bp, synthetic construct
  misc_feature 1..42: note = Synthetic
  source 1..42: mol_type = other DNA
gcgagagatgaggactggaattatgggagctggttcgacacc

SEQ ID NO: 72 - protein, 14 aa, synthetic construct
  REGION 1..14: note = Synthetic
ARDEDWNYGSWFDT

SEQ ID NO: 73 - DNA, 336 bp, synthetic construct
  misc_feature 1..336: note = Synthetic
  source 1..336: mol_type = other DNA
gatattgtaatgacccagactccactctcctcacctgtcacccttggacagccggcctccatctcctgcaggtctagtccaagcctcgtacacagtgatggaaacacctacttgagttggcttcagcagaggccaggccagcctccaagactcctaatttataagatttctaaccgattctctggggtcccagacagattcagtggcagtggggcagggacagatttcacgctgaaaatcagcagggtggaagctgaggatgtcggggtttattactgcatgcaagctacacattttccgatcaccttcggccaagggacacgactggagattaga

SEQ ID NO: 74 - protein, 112 aa, synthetic construct
  REGION 1..112: note = Synthetic
DIVMTQTPLSSPVTLGQPASISCRSSPSLVHSDGNTYLSWLQQRPGQPPRLLIYKISNRFSGVPDRFSGSGAGTDFTLKISRVEAEDVGVYYCMQATHFPITFGQGTRLEIR

SEQ ID NO: 75 - DNA, 33 bp, synthetic construct
  misc_feature 1..33: note = Synthetic
  source 1..33: mol_type = other DNA
ccaagcctcgtacacagtgatggaaacacctac

SEQ ID NO: 76 - protein, 11 aa, synthetic construct
  REGION 1..11: note = Synthetic
PSLVHSDGNTY

SEQ ID NO: 77-78 - skipped (2 entries)

SEQ ID NO: 79 - DNA, 27 bp, synthetic construct
  misc_feature 1..27: note = Synthetic
  source 1..27: mol_type = other DNA
atgcaagctacacattttccgatcacc

SEQ ID NO: 80 - protein, 9 aa, synthetic construct
  REGION 1..9: note = Synthetic
MQATHFPIT

SEQ ID NO: 81 - DNA, 360 bp, synthetic construct
  misc_feature 1..360: note = Synthetic
  source 1..360: mol_type = other DNA
gaggtgcagctggtggaatctgggggaggtgtggtgcggcctggggggtccctgagactctcctgtgcagcctctggattcacttttgatgattatggcatgacctgggtccgccaagctccagggaggggcctggaatgggtctctggtattcattggcatggtaaacgcacaggttatgcagactctgtgaagggccgattcaccatatccagagacaacgccaagaaatccctgtatctgcaaatgaacagtctgaaaggcgaggacacggccttgtatcattgtgtgagggggggaatgagtacaggggactggttcgacccctggggccagggaaccctggtcatcgtctcctca

SEQ ID NO: 82 - protein, 120 aa, synthetic construct
  REGION 1..120: note = Synthetic
EVQLVESGGGVVRPGGSLRLSCAASGFTFDDYGMTWVRQAPGRGLEWVSGIHWHGKRTGYADSVKGRFTISRDNAKKSLYLQMNSLKGEDTALYHCVRGGMSTGDWFDPWGQGTLVIVSS

SEQ ID NO: 83 - DNA, 24 bp, synthetic construct
  misc_feature 1..24: note = Synthetic
  source 1..24: mol_type = other DNA
ggattcacttttgatgattatggc

SEQ ID NO: 84 - protein, 8 aa, synthetic construct
  REGION 1..8: note = Synthetic
GFTFDDYG

SEQ ID NO: 85 - DNA, 24 bp, synthetic construct
  misc_feature 1..24: note = Synthetic
  source 1..24: mol_type = other DNA
attcattggcatggtaaacgcaca

SEQ ID NO: 86 - protein, 8 aa, synthetic construct
  REGION 1..8: note = Synthetic
IHWHGKRT

SEQ ID NO: 87 - DNA, 39 bp, synthetic construct
  misc_feature 1..39: note = Synthetic
  source 1..39: mol_type = other DNA
gtgagggggggaatgagtacaggggactggttcgacccc

SEQ ID NO: 88 - protein, 13 aa, synthetic construct
  REGION 1..13: note = Synthetic
VRGGMSTGDWFDP

SEQ ID NO: 89 - DNA, 324 bp, synthetic construct
  misc_feature 1..324: note = Synthetic
  source 1..324: mol_type = other DNA
gacatccagatgacccagtctccatcctccctgtctgcatctctaggagacagagtcaccatcacttgccgggcaagtcagagcattaacagttatttaaattggtatcagcagaaaccagggaaagcccctaaactcctgatctatgttgcatccagtttgcaaagtggggtcccatcaaggttcagtggcagtggatctgggacagaattcactctcaccatcagcaatctgcaacctgaagattttgcaacttactactgtcaacagagttacagtacccctccgatcaccttcggccaagggacacgactggagattaaa

SEQ ID NO: 90 - protein, 108 aa, synthetic construct
  REGION 1..108: note = Synthetic
DIQMTQSPSSLSASLGDRVTITCRASQSINSYLNWYQQKPGKAPKLLIYVASSLQSGVPSRFSGSGSGTEFTLTISNLQPEDFATYYCQQSYSTPPITFGQGTRLEIK

SEQ ID NO: 91 - DNA, 18 bp, synthetic construct
  misc_feature 1..18: note = Synthetic
  source 1..18: mol_type = other DNA
cagagcattaacagttat

SEQ ID NO: 92 - protein, 6 aa, synthetic construct
  REGION 1..6: note = Synthetic
QSINSY

SEQ ID NO: 93-94 - skipped (2 entries)

SEQ ID NO: 95 - DNA, 30 bp, synthetic construct
  misc_feature 1..30: note = Synthetic
  source 1..30: mol_type = other DNA
caacagagttacagtacccctccgatcacc

SEQ ID NO: 96 - protein, 10 aa, synthetic construct
  REGION 1..10: note = Synthetic
QQSYSTPPIT

SEQ ID NO: 97 - DNA, 360 bp, synthetic construct
  misc_feature 1..360: note = Synthetic
  source 1..360: mol_type = other DNA
gaggtgcagctggtggagtctgggggaggtgtggtacggccgggggggtccctgagactctcctgtgcagcctctggattcacctttgatgattatggcatgacctgggtccgccaagttccagggaaggggctggagtgggtctctggtattcattggagtggtagaagcacaggttatgcagactctgtgaagggccgattcaccatctccagagacaacgccaagaactccctgtatctgcaaatgaacagtctgagagccgaggacacggccttgtattactgtgcgagggggggaatgagtacgggggactggttcgacccctggggccagggaaccctggtcaccgtctcctca

SEQ ID NO: 98 - protein, 120 aa, synthetic construct
  REGION 1..120: note = Synthetic
EVQLVESGGGVVRPGGSLRLSCAASGFTFDDYGMTWVRQVPGKGLEWVSGIHWSGRSTGYADSVKGRFTISRDNAKNSLYLQMNSLRAEDTALYYCARGGMSTGDWFDPWGQGTLVTVSS

SEQ ID NO: 99 - DNA, 24 bp, synthetic construct
  misc_feature 1..24: note = Synthetic
  source 1..24: mol_type = other DNA
ggattcacctttgatgattatggc

SEQ ID NO: 100 - protein, 8 aa, synthetic construct
  REGION 1..8: note = Synthetic
GFTFDDYG

SEQ ID NO: 101 - DNA, 24 bp, synthetic construct
  misc_feature 1..24: note = Synthetic
  source 1..24: mol_type = other DNA
attcattggagtggtagaagcaca

SEQ ID NO: 102 - protein, 8 aa, synthetic construct
  REGION 1..8: note = Synthetic
IHWSGRST

SEQ ID NO: 103 - DNA, 39 bp, synthetic construct
  misc_feature 1..39: note = Synthetic
  source 1..39: mol_type = other DNA
gcgagggggggaatgagtacgggggactggttcgacccc

SEQ ID NO: 104 - protein, 13 aa, synthetic construct
  REGION 1..13: note = Synthetic
ARGGMSTGDWFDP

SEQ ID NO: 105 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgttgcatccagtttgcaaagtggggtcccatcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 106 - protein, 108 aa, synthetic construct
  REGION 1..108: note = Synthetic
DIQMTQSPSSLSASVGDRVTITCRASQSISSYLNWYQQKPGKAPKLLIYVASSLQSGVPSRFSGSGSGTDFTLTISSLQPEDFATYYCQQSYSTPPITFGQGTRLEIK

SEQ ID NO: 107 - DNA, 18 bp, synthetic construct
  misc_feature 1..18: note = Synthetic
  source 1..18: mol_type = other DNA
cagagcattagcagctat

SEQ ID NO: 108 - protein, 6 aa, synthetic construct
  REGION 1..6: note = Synthetic
QSISSY

SEQ ID NO: 109-110 - skipped (2 entries)

SEQ ID NO: 111 - DNA, 30 bp, synthetic construct; identical to SEQ ID NO: 95
  misc_feature 1..30: note = Synthetic
  source 1..30: mol_type = other DNA

SEQ ID NO: 112 - protein, 10 aa, synthetic construct
  REGION 1..10: note = Synthetic
QQSYSTPPIT

SEQ ID NO: 113 - DNA, 345 bp, synthetic construct
  misc_feature 1..345: note = Synthetic
  source 1..345: mol_type = other DNA
gaggtgcagttggtggagtctggaggaggcttggtccagcctggggggtccctaagactctcctgtgcagcctctgggttcaccgtcggtagtaactacatgaactgggtccgtcaggctccagggaagggactggagtgggtctcagttatttatagtggtggtagtacatactacgcagattccgtgaagggccgattcaccatctccagactcacttccaagaacacactgtatcttcaaatgagcagcctgagacctgaggacacggccgtgtattattgtgcgagagggattaggggtctggacgtctggggccaagggaccacggtcaccgtctcttca

SEQ ID NO: 114 - protein, 115 aa, synthetic construct
  REGION 1..115: note = Synthetic
EVQLVESGGGLVQPGGSLRLSCAASGFTVGSNYMNWVRQAPGKGLEWVSVIYSGGSTYYADSVKGRFTISRLTSKNTLYLQMSSLRPEDTAVYYCARGIRGLDVWGQGTTVTVSS

SEQ ID NO: 115 - DNA, 24 bp, synthetic construct
  misc_feature 1..24: note = Synthetic
  source 1..24: mol_type = other DNA
gggttcaccgtcggtagtaactac

SEQ ID NO: 116 - protein, 8 aa, synthetic construct
  REGION 1..8: note = Synthetic
GFTVGSNY

SEQ ID NO: 117 - DNA, 21 bp, synthetic construct
  misc_feature 1..21: note = Synthetic
  source 1..21: mol_type = other DNA
atttatagtggtggtagtaca

SEQ ID NO: 118 - protein, 7 aa, synthetic construct
  REGION 1..7: note = Synthetic
IYSGGST

SEQ ID NO: 119 - DNA, 27 bp, synthetic construct
  misc_feature 1..27: note = Synthetic
  source 1..27: mol_type = other DNA
gcgagagggattaggggtctggacgtc

SEQ ID NO: 120 - protein, 9 aa, synthetic construct
  REGION 1..9: note = Synthetic
ARGIRGLDV

SEQ ID NO: 121 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagaccattaacatctatttaaattggtatcagcagaaaccagggagagcccctaggctcctgatctatgctgcatccagtttgcaaagtggggtcccatcaaggttcagtggcagtggatctgggacagatttcactctcaccatcagcagtctgcaacctgaagattttgcaacttactactgtcaccagagttacagtacccctccgatcaccttcggccaagggacacgactggagattaaa

SEQ ID NO: 122 - protein, 108 aa, synthetic construct
  REGION 1..108: note = Synthetic
DIQMTQSPSSLSASVGDRVTITCRASQTINIYLNWYQQKPGRAPRLLIYAASSLQSGVPSRFSGSGSGTDFTLTISSLQPEDFATYYCHQSYSTPPITFGQGTRLEIK

SEQ ID NO: 123 - DNA, 18 bp, synthetic construct
  misc_feature 1..18: note = Synthetic
  source 1..18: mol_type = other DNA
cagaccattaacatctat

SEQ ID NO: 124 - protein, 6 aa, synthetic construct
  REGION 1..6: note = Synthetic
QTINIY

SEQ ID NO: 125-126 - skipped (2 entries)

SEQ ID NO: 127 - DNA, 30 bp, synthetic construct
  misc_feature 1..30: note = Synthetic
  source 1..30: mol_type = other DNA
caccagagttacagtacccctccgatcacc

SEQ ID NO: 128 - protein, 10 aa, synthetic construct
  REGION 1..10: note = Synthetic
HQSYSTPPIT

SEQ ID NO: 129 - DNA, 345 bp, synthetic construct
  misc_feature 1..345: note = Synthetic
  source 1..345: mol_type = other DNA
gaggaacggttggtggagtctggaggagacttggtccagcctggggggtccctgagactctcctgtgcagcctctggcatcaccgtcggtactaattatatgaactgggtccgccaggctccagggaagggactggagtgggtctcagttatttctagcggtggtaatacacactacgcagactccgtgaagggccgattcattatgtccagacaaacttccaaaaacacgctgtatcttcagatgaatagcctggaaactgaggacacggccgtatattattgtgcgagggggatcagaggtttggacgtctggggccaagggaccatggtcaccgtctcctca

SEQ ID NO: 130 - protein, 115 aa, synthetic construct
  REGION 1..115: note = Synthetic
EERLVESGGDLVQPGGSLRLSCAASGITVGTNYMNWVRQAPGKGLEWVSVISSGGNTHYADSVKGRFIMSRQTSKNTLYLQMNSLETEDTAVYYCARGIRGLDVWGQGTMVTVSS

SEQ ID NO: 131 - DNA, 24 bp, synthetic construct
  misc_feature 1..24: note = Synthetic
  source 1..24: mol_type = other DNA
ggcatcaccgtcggtactaattat

SEQ ID NO: 132 - protein, 8 aa, synthetic construct
  REGION 1..8: note = Synthetic
GITVGTNY

SEQ ID NO: 133 - DNA, 21 bp, synthetic construct
  misc_feature 1..21: note = Synthetic
  source 1..21: mol_type = other DNA
atttctagcggtggtaataca

SEQ ID NO: 134 - protein, 7 aa, synthetic construct
  REGION 1..7: note = Synthetic
ISSGGNT

SEQ ID NO: 135 - DNA, 27 bp, synthetic construct
  misc_feature 1..27: note = Synthetic
  source 1..27: mol_type = other DNA
gcgagggggatcagaggtttggacgtc

SEQ ID NO: 136 - protein, 9 aa, synthetic construct
  REGION 1..9: note = Synthetic
ARGIRGLDV

SEQ ID NO: 137 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcatgagcagctatttaaattggtatcagcagaaaccagggagagcccctaagctcctgatctttgctgcatccagtttgcaaagtggggtcccatcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 138 - protein, 108 aa, synthetic construct
  REGION 1..108: note = Synthetic
DIQMTQSPSSLSASVGDRVTITCRASQSMSSYLNWYQQKPGRAPKLLIFAASSLQSGVPSRFSGSGSGTDFTLTISSLQPEDFATYYCQQSYSTPPITFGQGTRLEIK

SEQ ID NO: 139 - DNA, 18 bp, synthetic construct
  misc_feature 1..18: note = Synthetic
  source 1..18: mol_type = other DNA
cagagcatgagcagctat

SEQ ID NO: 140 - protein, 6 aa, synthetic construct
  REGION 1..6: note = Synthetic
QSMSSY

SEQ ID NO: 141-142 - skipped (2 entries)

SEQ ID NO: 143 - DNA, 30 bp, synthetic construct; identical to SEQ ID NO: 95
  misc_feature 1..30: note = Synthetic
  source 1..30: mol_type = other DNA

SEQ ID NO: 144 - protein, 10 aa, synthetic construct
  REGION 1..10: note = Synthetic
QQSYSTPPIT

SEQ ID NO: 145 - DNA, 354 bp, synthetic construct
  misc_feature 1..354: note = Synthetic
  source 1..354: mol_type = other DNA
caggtccagctggtgcagtctggggctgaggtgaagatgcctgggtcctcggtgagggtctcctgcaaggcttctggaggcatcttcagcagttctactatcagttgggtgcgacaggcccctggacaagggcttgaatggatgggagagatcatccctgtctttggtacagtaaactacgcacagaagttccaggacagagtcatatttaccgcggacgaatctacgactacagcctacatggagctgagcagcctgaaatctggggacacggccgtatatttctgtgcgcgaaattggggattaggctctttttatatctggggccaagggacaatggtcaccgtctcttca

SEQ ID NO: 146 - protein, 118 aa, synthetic construct
  REGION 1..118: note = Synthetic
QVQLVQSGAEVKMPGSSVRVSCKASGGIFSSSTISWVRQAPGQGLEWMGEIIPVFGTVNYAQKFQDRVIFTADESTTTAYMELSSLKSGDTAVYFCARNWGLGSFYIWGQGTMVTVSS

SEQ ID NO: 147 - DNA, 24 bp, synthetic construct
  misc_feature 1..24: note = Synthetic
  source 1..24: mol_type = other DNA
ggaggcatcttcagcagttctact

SEQ ID NO: 148 - protein, 8 aa, synthetic construct
  REGION 1..8: note = Synthetic
GGIFSSST

SEQ ID NO: 149 - DNA, 24 bp, synthetic construct
  misc_feature 1..24: note = Synthetic
  source 1..24: mol_type = other DNA
atcatccctgtctttggtacagta

SEQ ID NO: 150 - protein, 8 aa, synthetic construct
  REGION 1..8: note = Synthetic
IIPVFGTV

SEQ ID NO: 151 - DNA, 33 bp, synthetic construct
  misc_feature 1..33: note = Synthetic
  source 1..33: mol_type = other DNA
gcgcgaaattggggattaggctctttttatatc

SEQ ID NO: 152 - protein, 11 aa, synthetic construct
  REGION 1..11: note = Synthetic
ARNWGLGSFYI

SEQ ID NO: 153 - DNA, 324 bp, synthetic construct
  misc_feature 1..324: note = Synthetic
  source 1..324: mol_type = other DNA
gaaattgtgttgacgcagtctccaggcaccctgtctttgtctccaggggaaagagccaccctctcctgcagggccagtcagagttttaacttcaactacttagcctggtaccagcagaaacctggccaggctcccagactcctcatctatggtgcatccagcagggccactggcatcccagacaggttcagtggcagtgggtctgggacagacttcactctcaccatcaacaggctggagcctgaagattttggagtgttttattgtcagcagtatgaaagcgcaccttggacgttcggccaagggaccaaggtggaaatcaaa

SEQ ID NO: 154 - protein, 108 aa, synthetic construct
  REGION 1..108: note = Synthetic
EIVLTQSPGTLSLSPGERATLSCRASQSFNFNYLAWYQQKPGQAPRLLIYGASSRATGIPDRFSGSGSGTDFTLTINRLEPEDFGVFYCQQYESAPWTFGQGTKVEIK

SEQ ID NO: 155 - DNA, 21 bp, synthetic construct
  misc_feature 1..21: note = Synthetic
  source 1..21: mol_type = other DNA
cagagttttaacttcaactac

SEQ ID NO: 156 - protein, 7 aa, synthetic construct
  REGION 1..7: note = Synthetic
QSFNFNY

SEQ ID NO: 157-158 - skipped (2 entries)

SEQ ID NO: 159 - DNA, 27 bp, synthetic construct
  misc_feature 1..27: note = Synthetic
  source 1..27: mol_type = other DNA
cagcagtatgaaagcgcaccttggacg

SEQ ID NO: 160 - protein, 9 aa, synthetic construct
  REGION 1..9: note = Synthetic
QQYESAPWT

SEQ ID NO: 161 - DNA, 345 bp, synthetic construct
  misc_feature 1..345: note = Synthetic
  source 1..345: mol_type = other DNA
gaggtgcagcttgtagagtctgggggagacttggtacatcctggcaggtccctgagactctcctgtgcagcctctggtttcccctttgatgagtatgccatgcactgggtccggcaagttccagggaagggcctggagtgggtctcaggtattagttggagtaataataacataggctatgcggactctgtgaagggccgattcaccatctccagagacaacgccaaaaactccctgtatctacaaatgaacagtctgagacctgaggacacggccttttattactgtgcaaaatctggaatctttgactcctggggccagggaaccctggtcaccgtctcctca

SEQ ID NO: 162 - protein, 115 aa, synthetic construct
  REGION 1..115: note = Synthetic
EVQLVESGGDLVHPGRSLRLSCAASGFPFDEYAMHWVRQVPGKGLEWVSGISWSNNNIGYADSVKGRFTISRDNAKNSLYLQMNSLRPEDTAFYYCAKSGIFDSWGQGTLVTVSS

SEQ ID NO: 163 - DNA, 24 bp, synthetic construct
  misc_feature 1..24: note = Synthetic
  source 1..24: mol_type = other DNA
ggtttcccctttgatgagtatgcc

SEQ ID NO: 164 - protein, 8 aa, synthetic construct
  REGION 1..8: note = Synthetic
GFPFDEYA

SEQ ID NO: 165 - DNA, 24 bp, synthetic construct
  misc_feature 1..24: note = Synthetic
  source 1..24: mol_type = other DNA
attagttggagtaataataacata

SEQ ID NO: 166 - protein, 8 aa, synthetic construct
  REGION 1..8: note = Synthetic
ISWSNNNI

SEQ ID NO: 167 - DNA, 24 bp, synthetic construct
  misc_feature 1..24: note = Synthetic
  source 1..24: mol_type = other DNA
gcaaaatctggaatctttgactcc

SEQ ID NO: 168 - protein, 8 aa, synthetic construct
  REGION 1..8: note = Synthetic
AKSGIFDS

SEQ ID NO: 169 - DNA, 315 bp, synthetic construct
  misc_feature 1..315: note = Synthetic
  source 1..315: mol_type = other DNA
gacatccagatgacccagtctccatcctccctgtctgcatctgtaggagacagagtcaccatcacttgccgggcaagtcagagcattagcagctatttaaattggtatcagcagaaaccagggaagctcctgatctatgctgcatccagtttgcaaagtggggtcccatcacggttcagtggcggtggatctgggacagatttcactctcaccatcagcagtctgcgacctgaagattttgcaacttactactgtcaacagagttactgtacccctccgatcaccttcggccaagggacacgactggagattaaa

SEQ ID NO: 170 - protein, 105 aa, synthetic construct
  REGION 1..105: note = Synthetic
DIQMTQSPSSLSASVGDRVTITCRASQSISSYLNWYQQKPGKLLIYAASSLQSGVPSRFSGGGSGTDFTLTISSLRPEDFATYYCQQSYCTPPITFGQGTRLEIK

SEQ ID NO: 171 - DNA, 18 bp, synthetic construct
  misc_feature 1..18: note = Synthetic
  source 1..18: mol_type = other DNA
cagagcattagcagctat

SEQ ID NO: 172 - protein, 6 aa, synthetic construct
  REGION 1..6: note = Synthetic
QSISSY

SEQ ID NO: 173-174 - skipped (2 entries)

SEQ ID NO: 175 - DNA, 30 bp, synthetic construct
  misc_feature 1..30: note = Synthetic
  source 1..30: mol_type = other DNA
caacagagttactgtacccctccgatcacc

SEQ ID NO: 176 - protein, 10 aa, synthetic construct
  REGION 1..10: note = Synthetic
QQSYCTPPIT

SEQ ID NO: 177 - DNA, 354 bp, synthetic construct
  misc_feature 1..354: note = Synthetic
  source 1..354: mol_type = other DNA
gaggtgcagctggtggagtccgggggaggcgtggtccagcctgggaggtccctgagactctcctgtgcagcctctggattcaccttcagtagttatggcatgcactgggtccgccaggctccaggcaagggactggagtgggtgacacttatatcatatgagggaaggaataaatactatgcagactccgtgaagggccgattcaccatttccagagacaattccaagaacacgctgtatctgcaaatgaacagcctgagagctgaggacacggctgtatattactgtgcgaaagataggaccctttacggtatggacgtctggggccaaggaaccacggtcaccgtctcctca

SEQ ID NO: 178 - protein, 118 aa, synthetic construct
  REGION 1..118: note = Synthetic
EVQLVESGGGVVQPGRSLRLSCAASGFTFSSYGMHWVRQAPGKGLEWVTLISYEGRNKYYADSVKGRFTISRDNSKNTLYLQMNSLRAEDTAVYYCAKDRTLYGMDVWGQGTTVTVSS

SEQ ID NO: 179 - DNA, 24 bp, synthetic construct
  misc_feature 1..24: note = Synthetic
  source 1..24: mol_type = other DNA
ggattcaccttcagtagttatggc

SEQ ID NO: 180 - protein, 8 aa, synthetic construct
  REGION 1..8: note = Synthetic
GFTFSSYG

SEQ ID NO: 181 - DNA, 24 bp, synthetic construct
  misc_feature 1..24: note = Synthetic
  source 1..24: mol_type = other DNA
atatcatatgagggaaggaataaa

SEQ ID NO: 182 - protein, 8 aa, synthetic construct
  REGION 1..8: note = Synthetic
ISYEGRNK

SEQ ID NO: 183 - DNA, 33 bp, synthetic construct
  misc_feature 1..33: note = Synthetic
  source 1..33: mol_type = other DNA
gcgaaagataggaccctttacggtatggacgtc

SEQ ID NO: 184 - protein, 11 aa, synthetic construct
  REGION 1..11: note = Synthetic
AKDRTLYGMDV

SEQ ID NO: 185 - DNA, 363 bp, synthetic construct
  misc_feature 1..363: note = Synthetic
  source 1..363: mol_type = other DNA
caggtcaccttgagggagtctggtcctgcgctggtgaaaaccacacagaccctcacactgacctgcaccttctctgggttctcactcagcactaatagaatgtgtgtgacctggatccgtcagcccccagggaaggccctggagtggcttgcgcgcattgattgggatggtgttaaatactacaacacatctctgaagaccaggctcaccatctccaaggacacctccaaaaaccaggtggtccttacaatgaccaacatggaccctgtggacacagccactttttactgtgcacggtcgacttcgttgactttttactactttgactactggggccagggaaccctggtcaccgtctcctca

SEQ ID NO: 186 - protein, 121 aa, synthetic construct
  REGION 1..121: note = Synthetic
QVTLRESGPALVKTTQTLTLTCTFSGFSLSTNRMCVTWIRQPPGKALEWLARIDWDGVKYYNTSLKTRLTISKDTSKNQVVLTMTNMDPVDTATFYCARSTSLTFYYFDYWGQGTLVTVSS

SEQ ID NO: 187 - DNA, 30 bp, synthetic construct
  misc_feature 1..30: note = Synthetic
  source 1..30: mol_type = other DNA
gggttctcactcagcactaatagaatgtgt

SEQ ID NO: 188 - protein, 10 aa, synthetic construct
  REGION 1..10: note = Synthetic
GFSLSTNRMC

SEQ ID NO: 189 - DNA, 21 bp, synthetic construct
  misc_feature 1..21: note = Synthetic
  source 1..21: mol_type = other DNA
attgattgggatggtgttaaa

SEQ ID NO: 190 - protein, 7 aa, synthetic construct
  REGION 1..7: note = Synthetic
IDWDGVK

SEQ ID NO: 191 - DNA, 39 bp, synthetic construct
  misc_feature 1..39: note = Synthetic
  source 1..39: mol_type = other DNA
gcacggtcgacttcgttgactttttactactttgactac

SEQ ID NO: 192 - protein, 13 aa, synthetic construct
  REGION 1..13: note = Synthetic
ARSTSLTFYYFDY

SEQ ID NO: 193 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 194 - protein, 108 aa, synthetic construct
  REGION 1..108: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 195 - DNA, 18 bp, synthetic construct
  misc_feature 1..18: note = Synthetic
  source 1..18: mol_type = other DNA
cagagcattagcagctat

SEQ ID NO: 196 - protein, 6 aa, synthetic construct
  REGION 1..6: note = Synthetic
QSISSY

SEQ ID NO: 197-198 - skipped (2 entries)

SEQ ID NO: 199 - DNA, 30 bp, synthetic construct; identical to SEQ ID NO: 95
  misc_feature 1..30: note = Synthetic
  source 1..30: mol_type = other DNA

SEQ ID NO: 200 - protein, 10 aa, synthetic construct
  REGION 1..10: note = Synthetic
QQSYSTPPIT

SEQ ID NO: 201 - DNA, 345 bp, synthetic construct
  misc_feature 1..345: note = Synthetic
  source 1..345: mol_type = other DNA
gaggtgcagctggtggagtctggaggaggcttggtccagccgggggggtccctgagactctcctgtgcagcctctgagttcaccgtcggtaccaaccacatgaactgggtccgccaggctccagggaagggactggagtgggtctcagttatttatagcggtggtaacacattctacgcagactccgtgaagggccgattcaccatctccagacacacttccaagaacacgctgtatcttcaaatgaacagcctgacagcagaggacacggccgtatattactgtgcgcgaggattggggggtatggacgtctggggccaagggaccacggtcaccgtctcctca

SEQ ID NO: 202 - protein, 115 aa, synthetic construct
  REGION 1..115: note = Synthetic
EVQLVESGGGLVQPGGSLRLSCAASEFTVGTNHMNWVRQAPGKGLEWVSVIYSGGNTFYADSVKGRFTISRHTSKNTLYLQMNSLTAEDTAVYYCARGLGGMDVWGQGTTVTVSS

SEQ ID NO: 203 - DNA, 24 bp, synthetic construct
  misc_feature 1..24: note = Synthetic
  source 1..24: mol_type = other DNA
gagttcaccgtcggtaccaaccac

SEQ ID NO: 204 - protein, 8 aa, synthetic construct
  REGION 1..8: note = Synthetic
EFTVGTNH

SEQ ID NO: 205 - DNA, 21 bp, synthetic construct
  misc_feature 1..21: note = Synthetic
  source 1..21: mol_type = other DNA
atttatagcggtggtaacaca

SEQ ID NO: 206 - protein, 7 aa, synthetic construct
  REGION 1..7: note = Synthetic
IYSGGNT

SEQ ID NO: 207 - DNA, 27 bp, synthetic construct
  misc_feature 1..27: note = Synthetic
  source 1..27: mol_type = other DNA
gcgcgaggattggggggtatggacgtc

SEQ ID NO: 208 - protein, 9 aa, synthetic construct
  REGION 1..9: note = Synthetic
ARGLGGMDV

SEQ ID NO: 209 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcgagtcaggtcattagcaattatttagcctggtatcagcagaaaccagggaaagttcctaggctcctgatctatgctgcatccactttgcaatcaggggtcccatctcgtttcagtggcagtggatctgggacagatttcactctcaccatcagcagcctgcagcctgaagatgttgcaacttattactgtcaaaagtataacagtgcccctcggacgttcggccaagggaccaaggtggaaatcaaa

SEQ ID NO: 210 - protein, 107 aa, synthetic construct
  REGION 1..107: note = Synthetic
DIQMTQSPSSLSASVGDRVTITCRASQVISNYLAWYQQKPGKVPRLLIYAASTLQSGVPSRFSGSGSGTDFTLTISSLQPEDVATYYCQKYNSAPRTFGQGTKVEIK

SEQ ID NO: 211 - DNA, 18 bp, synthetic construct
  misc_feature 1..18: note = Synthetic
  source 1..18: mol_type = other DNA
caggtcattagcaattat

SEQ ID NO: 212 - protein, 6 aa, synthetic construct
  REGION 1..6: note = Synthetic
QVISNY

SEQ ID NO: 213-214 - skipped (2 entries)

SEQ ID NO: 215 - DNA, 27 bp, synthetic construct
  misc_feature 1..27: note = Synthetic
  source 1..27: mol_type = other DNA
caaaagtataacagtgcccctcggacg

SEQ ID NO: 216 - protein, 9 aa, synthetic construct
  REGION 1..9: note = Synthetic
QKYNSAPRT

SEQ ID NO: 217 - DNA, 360 bp, synthetic construct
  misc_feature 1..360: note = Synthetic
  source 1..360: mol_type = other DNA
gaggtgcagctggtggagtctgggggaggcttggtccagcggggggagtccctgagactttactgtgcagcctctggattcacctttagtaaatattggatgaactgggtccgccaggctccagggaaggggctggagtgggtggccaacataaagggagatggaagtgagaaatactatgtggactctgtgaagggccggttcaccatctccagagacaacgccaagaactcactatatctacaaatgaacagcctgagagccgaggacacggctgtttattactgtgcgagagattattggggatcaggctactactttgacttctggggccagggaaccctggtcaccgtctcctca

SEQ ID NO: 218 - protein, 120 aa, synthetic construct
  REGION 1..120: note = Synthetic
EVQLVESGGGLVQRGESLRLYCAASGFTFSKYWMNWVRQAPGKGLEWVANIKGDGSEKYYVDSVKGRFTISRDNAKNSLYLQMNSLRAEDTAVYYCARDYWGSGYYFDFWGQGTLVTVSS

SEQ ID NO: 219 - DNA, 24 bp, synthetic construct
  misc_feature 1..24: note = Synthetic
  source 1..24: mol_type = other DNA
ggattcacctttagtaaatattgg

SEQ ID NO: 220 - protein, 8 aa, synthetic construct
  REGION 1..8: note = Synthetic
GFTFSKYW

SEQ ID NO: 221 - DNA, 24 bp, synthetic construct
  misc_feature 1..24: note = Synthetic
  source 1..24: mol_type = other DNA
ataaagggagatggaagtgagaaa

SEQ ID NO: 222 - protein, 8 aa, synthetic construct
  REGION 1..8: note = Synthetic
IKGDGSEK

SEQ ID NO: 223 - DNA, 39 bp, synthetic construct
  misc_feature 1..39: note = Synthetic
  source 1..39: mol_type = other DNA
gcgagagattattggggatcaggctactactttgacttc

SEQ ID NO: 224 - protein, 13 aa, synthetic construct
  REGION 1..13: note = Synthetic
ARDYWGSGYYFDF

SEQ ID NO: 225 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcaagtcagaacattaacaactatttaaattggtatcagcagaaaccagggaaagcccctaaactcctgatctatgctgcatccagtttccaaaatgcggtcccatcaaggttcagtggcagtggatctgggacagatttcactctcaccatcagcagtctgcaacctgaagattttgcaacttactactgtcaacagagttacaataccccgctcactttcggcggggggaccaaggtggagatcaaa

SEQ ID NO: 226 - protein, 107 aa, synthetic construct
  REGION 1..107: note = Synthetic
DIQMTQSPSSLSASVGDRVTITCRASQNINNYLNWYQQKPGKAPKLLIYAASSFQNAVPSRFSGSGSGTDFTLTISSLQPEDFATYYCQQSYNTPLTFGGGTKVEIK

SEQ ID NO: 227 - DNA, 18 bp, synthetic construct
  misc_feature 1..18: note = Synthetic
  source 1..18: mol_type = other DNA
cagaacattaacaactat

SEQ ID NO: 228 - protein, 6 aa, synthetic construct
  REGION 1..6: note = Synthetic
QNINNY

SEQ ID NO: 229-230 - skipped (2 entries)

SEQ ID NO: 231 - DNA, 27 bp, synthetic construct
  misc_feature 1..27: note = Synthetic
  source 1..27: mol_type = other DNA
caacagagttacaataccccgctcact

SEQ ID NO: 232 - protein, 9 aa, synthetic construct
  REGION 1..9: note = Synthetic
QQSYNTPLT

SEQ ID NO: 233 - DNA, 390 bp, synthetic construct
  misc_feature 1..390: note = Synthetic
  source 1..390: mol_type = other DNA
gaggtgcagctggtggagtctgggggaggcttggtccagtctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgatgacacggctgtgtattactgtgcgagagatgatattgtagtagtaccagctcctatgggatattactactactacttcggtatggacgtctggggccaagggaccacggtcaccgtctcctca

SEQ ID NO: 234 - protein, 130 aa, synthetic construct
  REGION 1..130: note = Synthetic
EVQLVESGGGLVQSGGSLRLSCAASGFTFSSYWMSWVRQAPGKGLEWVANIKQDGSEKYYVDSVKGRFTISRDNAKNSLYLQMNSLRADDTAVYYCARDDIVVVPAPMGYYYYYFGMDVWGQGTTVTVSS

SEQ ID NO: 235 - DNA, 24 bp, synthetic construct
  misc_feature 1..24: note = Synthetic
  source 1..24: mol_type = other DNA
ggattcacctttagtagctattgg

SEQ ID NO: 236 - protein, 8 aa, synthetic construct
  REGION 1..8: note = Synthetic
GFTFSSYW

SEQ ID NO: 237 - DNA, 24 bp, synthetic construct
  misc_feature 1..24: note = Synthetic
  source 1..24: mol_type = other DNA
ataaagcaagatggaagtgagaaa

SEQ ID NO: 238 - protein, 8 aa, synthetic construct
  REGION 1..8: note = Synthetic
IKQDGSEK

SEQ ID NO: 239 - DNA, 69 bp, synthetic construct
  misc_feature 1..69: note = Synthetic
  source 1..69: mol_type = other DNA
gcgagagatgatattgtagtagtaccagctcctatgggatattactactactacttcggtatggacgtc

SEQ ID NO: 240 - protein, 23 aa, synthetic construct
  REGION 1..23: note = Synthetic
ARDDIVVVPAPMGYYYYYFGMDV

SEQ ID NO: 241 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtacacttttggccaggggaccaagctggagatcaaa

SEQ ID NO: 242 - protein, 107 aa, synthetic construct
  REGION 1..107: note = Synthetic
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKLEIK

SEQ ID NO: 243 - DNA, 18 bp, synthetic construct
  misc_feature 1..18: note = Synthetic
  source 1..18: mol_type = other DNA
cagggcattagaaatgat

SEQ ID NO: 244 - protein, 6 aa, synthetic construct
  REGION 1..6: note = Synthetic
QGIRND

SEQ ID NO: 245-246 - skipped (2 entries)

SEQ ID NO: 247 - DNA, 27 bp, synthetic construct
  misc_feature 1..27: note = Synthetic
  source 1..27: mol_type = other DNA
ctacagcataatagttacccgtacact

SEQ ID NO: 248 - protein, 9 aa, synthetic construct
  REGION 1..9: note = Synthetic
LQHNSYPYT

SEQ ID NO: 249 - DNA, 369 bp, synthetic construct
  misc_feature 1..369: note = Synthetic
  source 1..369: mol_type = other DNA
gaagtgcagctggtggagtctgggggaggcttggttcagcctggcaggtccctgagactctcctgtgcagcctctggattcacctttgatgattttgccatgcactgggtccgacaagctccagggaagggcctggagtgggtctcaggtattagttggactggtggtaacatggactatgcgaactctgtgaagggccgattcaccatctccagagaggacgccaagaattccctgtatctgcaaatgaacagtctgagagctgcggacacggccttgtattactgtgtaaaagatataagggggatagtggctacggggggggcttttgatatctggggccgagggacaatggtcaccgtctcttca

SEQ ID NO: 250 - protein, 123 aa, synthetic construct
  REGION 1..123: note = Synthetic
EVQLVESGGGLVQPGRSLRLSCAASGFTFDDFAMHWVRQAPGKGLEWVSGISWTGGNMDYANSVKGRFTISREDAKNSLYLQMNSLRAADTALYYCVKDIRGIVATGGAFDIWGRGTMVTVSS

SEQ ID NO: 251 - DNA, 24 bp, synthetic construct
  misc_feature 1..24: note = Synthetic
  source 1..24: mol_type = other DNA
ggattcacctttgatgattttgcc

SEQ ID NO: 252 - protein, 8 aa, synthetic construct
  REGION 1..8: note = Synthetic
GFTFDDFA

SEQ ID NO: 253 - DNA, 24 bp, synthetic construct
  misc_feature 1..24: note = Synthetic
  source 1..24: mol_type = other DNA
attagttggactggtggtaacatg

SEQ ID NO: 254 - protein, 8 aa, synthetic construct
  REGION 1..8: note = Synthetic
ISWTGGNM

SEQ ID NO: 255 - DNA, 48 bp, synthetic construct
  misc_feature 1..48: note = Synthetic
  source 1..48: mol_type = other DNA
gtaaaagatataagggggatagtggctacggggggggcttttgatatc

SEQ ID NO: 256 - protein, 16 aa, synthetic construct
  REGION 1..16: note = Synthetic
VKDIRGIVATGGAFDI

SEQ ID NO: 257 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatctcttgccgggcaagtcagaccattagcacttatttaaattggtttcagcagaaaccagggaaagcccctaagctcctgatctatgttgtgtccagtttgcaaagtggggtcccatcaaggttcagtggcagtggatctgggacagatttcactctcaccatcagcagtctgcaacctgaagattttgcaacttattactgtcaacagagttacagtaccccattcactttcggccctgggaccaaagtggatatcaaa

SEQ ID NO: 258 - protein, 107 aa, synthetic construct
  REGION 1..107: note = Synthetic
DIQMTQSPSSLSASVGDRVTISCRASQTISTYLNWFQQKPGKAPKLLIYVVSSLQSGVPSRFSGSGSGTDFTLTISSLQPEDFATYYCQQSYSTPFTFGPGTKVDIK

SEQ ID NO: 259 - DNA, 18 bp, synthetic construct
  misc_feature 1..18: note = Synthetic
  source 1..18: mol_type = other DNA
cagaccattagcacttat

SEQ ID NO: 260 - protein, 6 aa, synthetic construct
  REGION 1..6: note = Synthetic
QTISTY

SEQ ID NO: 261-262 - skipped (2 entries)

SEQ ID NO: 263 - DNA, 27 bp, synthetic construct
  misc_feature 1..27: note = Synthetic
  source 1..27: mol_type = other DNA
caacagagttacagtaccccattcact

SEQ ID NO: 264 - protein, 9 aa, synthetic construct
  REGION 1..9: note = Synthetic
QQSYSTPFT

SEQ ID NO: 265 - DNA, 345 bp, synthetic construct
  misc_feature 1..345: note = Synthetic
  source 1..345: mol_type = other DNA
gaggtgcagctggtggagtctggaggaggcttggtccagccgggggggtccctgagactctcctgtgcagcctctggattcaccgtcggtaccaactacatgaactgggtccgccaggctccagggaagggactggagtggatctcagttatttatagcggtggtagcacattctacgcagactccgtgaagggccgattcaccatctccagacagacttcccagaacacgctgtatcttcaaatgaacagcctgagacctgaggacacggccgtatattactgtgcgagaggtatacgtggttttgatatctggggccaagggacaatggtcaccgtctcttca

SEQ ID NO: 266 - protein, 115 aa, synthetic construct
  REGION 1..115: note = Synthetic
EVQLVESGGGLVQPGGSLRLSCAASGFTVGTNYMNWVRQAPGKGLEWISVIYSGGSTFYADSVKGRFTISRQTSQNTLYLQMNSLRPEDTAVYYCARGIRGFDIWGQGTMVTVSS

SEQ ID NO: 267 - DNA, 24 bp, synthetic construct
  misc_feature 1..24: note = Synthetic
  source 1..24: mol_type = other DNA
ggattcaccgtcggtaccaactac

SEQ ID NO: 268 - protein, 8 aa, synthetic construct
  REGION 1..8: note = Synthetic
GFTVGTNY

SEQ ID NO: 269 - DNA, 21 bp, synthetic construct
  misc_feature 1..21: note = Synthetic
  source 1..21: mol_type = other DNA
atttatagcggtggtagcaca

SEQ ID NO: 270 - protein, 7 aa, synthetic construct
  REGION 1..7: note = Synthetic
IYSGGST

SEQ ID NO: 271 - DNA, 27 bp, synthetic construct
  misc_feature 1..27: note = Synthetic
  source 1..27: mol_type = other DNA
gcgagaggtatacgtggttttgatatc

SEQ ID NO: 272 - protein, 9 aa, synthetic construct
  REGION 1..9: note = Synthetic
ARGIRGFDI

SEQ ID NO: 273 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 274 - protein, 108 aa, synthetic construct; identical to SEQ ID NO: 194
  REGION 1..108: note = Synthetic

SEQ ID NO: 275 - DNA, 18 bp, synthetic construct
  misc_feature 1..18: note = Synthetic
  source 1..18: mol_type = other DNA
cagagcattagcagctat

SEQ ID NO: 276 - protein, 6 aa, synthetic construct
  REGION 1..6: note = Synthetic
QSISSY

SEQ ID NO: 277-278 - skipped (2 entries)

SEQ ID NO: 279 - DNA, 30 bp, synthetic construct; identical to SEQ ID NO: 95
  misc_feature 1..30: note = Synthetic
  source 1..30: mol_type = other DNA

SEQ ID NO: 280 - protein, 10 aa, synthetic construct
  REGION 1..10: note = Synthetic
QQSYSTPPIT

SEQ ID NO: 281 - DNA, 345 bp, synthetic construct
  misc_feature 1..345: note = Synthetic
  source 1..345: mol_type = other DNA
gaggtgcagctggtggagtctggaggaggcttggtccagccgggggggtccctgagactctcctgtgcagcctctgggtttaccatcagtaccaactacatgaactgggtccgccaggctccagggaaggggctggagtgggtcgcagttatttatagcagtggttccacatactatatcgactccgtgaagggccgattcaccatctccagactcacttccaagaacacggtgtatcttcaaatgagcagcctgaattctgaagacacggccgtgtattactgtgcgagggggatcaggggttttgatatttggggccaagggacaatggtcaccgtctcttca

SEQ ID NO: 282 - protein, 115 aa, synthetic construct
  REGION 1..115: note = Synthetic
EVQLVESGGGLVQPGGSLRLSCAASGFTISTNYMNWVRQAPGKGLEWVAVIYSSGSTYYIDSVKGRFTISRLTSKNTVYLQMSSLNSEDTAVYYCARGIRGFDIWGQGTMVTVSS

SEQ ID NO: 283 - DNA, 24 bp, synthetic construct
  misc_feature 1..24: note = Synthetic
  source 1..24: mol_type = other DNA
gggtttaccatcagtaccaactac

SEQ ID NO: 284 - protein, 8 aa, synthetic construct
  REGION 1..8: note = Synthetic
GFTISTNY

SEQ ID NO: 285 - DNA, 21 bp, synthetic construct
  misc_feature 1..21: note = Synthetic
  source 1..21: mol_type = other DNA
atttatagcagtggttccaca

SEQ ID NO: 286 - protein, 7 aa, synthetic construct
  REGION 1..7: note = Synthetic
IYSSGST

SEQ ID NO: 287 - DNA, 27 bp, synthetic construct
  misc_feature 1..27: note = Synthetic
  source 1..27: mol_type = other DNA
gcgagggggatcaggggttttgatatt

SEQ ID NO: 288 - protein, 9 aa, synthetic construct
  REGION 1..9: note = Synthetic
ARGIRGFDI

SEQ ID NO: 289 - DNA, 372 bp, synthetic construct
  misc_feature 1..372: note = Synthetic
  source 1..372: mol_type = other DNA
gaagtgcagctggtggagtcggggggaggcttggtacagcctggcaggtccctgagactctcctgtgcagcctctggattcaccattgatgatagtgccatgcactgggtccggcaaactccagggaagggcctggagtgggtctcaggtattagttggaaaagtggtagcataggttatgcggactctgtgaggggccgattcaccatctccagagacaacgccaagaattccctctatctgcaaatgaacagtctgagagttgaggacacggccttgtattactgtgtaaaagatataaggggcaactggaactacgggggaaactggttcgacccctggggccagggaaccctggtcactgtctcctca

SEQ ID NO: 290 - protein, 124 aa, synthetic construct
  REGION 1..124: note = Synthetic
EVQLVESGGGLVQPGRSLRLSCAASGFTIDDSAMHWVRQTPGKGLEWVSGISWKSGSIGYADSVRGRFTISRDNAKNSLYLQMNSLRVEDTALYYCVKDIRGNWNYGGNWFDPWGQGTLVTVSS

SEQ ID NO: 291 - DNA, 24 bp, synthetic construct
  misc_feature 1..24: note = Synthetic
  source 1..24: mol_type = other DNA
ggattcaccattgatgatagtgcc

SEQ ID NO: 292 - protein, 8 aa, synthetic construct
  REGION 1..8: note = Synthetic
GFTIDDSA

SEQ ID NO: 293 - DNA, 24 bp, synthetic construct
  misc_feature 1..24: note = Synthetic
  source 1..24: mol_type = other DNA
attagttggaaaagtggtagcata

SEQ ID NO: 294 - protein, 8 aa, synthetic construct
  REGION 1..8: note = Synthetic
ISWKSGSI

SEQ ID NO: 295 - DNA, 51 bp, synthetic construct
  misc_feature 1..51: note = Synthetic
  source 1..51: mol_type = other DNA
gtaaaagatataaggggcaactggaactacgggggaaactggttcgacccc

SEQ ID NO: 296 - protein, 17 aa, synthetic construct
  REGION 1..17: note = Synthetic
VKDIRGNWNYGGNWFDP

SEQ ID NO: 297 - DNA, 345 bp, synthetic construct
  misc_feature 1..345: note = Synthetic
  source 1..345: mol_type = other DNA
gaggtgcagctggtggagtctggaggaggcttggtccagcctggggggtccctgagactctcatgtgaagcctctgggttcaccgtcggtgtcaaccacatgaactgggtccgccaggctccagggaagggtctggagtgggtctcagttattttcagtagtggtaggacattctacggagactacgtgaaggggcgattaaccatcttcagacaaacctcccagaacacggtgtatcttcaaatgaatagcctgagaagtgaggacacggccatatattactgtgcgagagggattggcggtttggacatctggggccgagggacaatggtcaccgtctcttca

SEQ ID NO: 298 - protein, 115 aa, synthetic construct
  REGION 1..115: note = Synthetic
EVQLVESGGGLVQPGGSLRLSCEASGFTVGVNHMNWVRQAPGKGLEWVSVIFSSGRTFYGDYVKGRLTIFRQTSQNTVYLQMNSLRSEDTAIYYCARGIGGLDIWGRGTMVTVSS

SEQ ID NO: 299 - DNA, 24 bp, synthetic construct
  misc_feature 1..24: note = Synthetic
  source 1..24: mol_type = other DNA
gggttcaccgtcggtgtcaaccac

SEQ ID NO: 300 - protein, 8 aa, synthetic construct
  REGION 1..8: note = Synthetic
GFTVGVNH

SEQ ID NO: 301 - DNA, 21 bp, synthetic construct
  misc_feature 1..21: note = Synthetic
  source 1..21: mol_type = other DNA
attttcagtagtggtaggaca

SEQ ID NO: 302 - protein, 7 aa, synthetic construct
  REGION 1..7: note = Synthetic
IFSSGRT

SEQ ID NO: 303 - DNA, 27 bp, synthetic construct
  misc_feature 1..27: note = Synthetic
  source 1..27: mol_type = other DNA
gcgagagggattggcggtttggacatc

SEQ ID NO: 304 - protein, 9 aa, synthetic construct
  REGION 1..9: note = Synthetic
ARGIGGLDI

SEQ ID NO: 305 - DNA, 369 bp, synthetic construct
  misc_feature 1..369: note = Synthetic
  source 1..369: mol_type = other DNA
gaagtgcagctggtggagtctgggggaggcttggttcagcctggcaggtccctaagactctcctgtgcagcctctggattcacctttgatgattatgccttgcactgggtccggcaagctccagggaagggcctggagtgggtctcaggtattagttggactggtggtactatagactatgcggactctgtgaagggccgattcaccatctccagagacaacgccaagaactccctgtatctgcaaatgagcagtctgagaactgaggacacggccatatattactgtacaagagatatccgggggaactggaagtacggaggctggttcgacccctggggccagggaaccctggtcaccgtctcctca

SEQ ID NO: 306 - protein, 123 aa, synthetic construct
  REGION 1..123: note = Synthetic
EVQLVESGGGLVQPGRSLRLSCAASGFTFDDYALHWVRQAPGKGLEWVSGISWTGGTIDYADSVKGRFTISRDNAKNSLYLQMSSLRTEDTAIYYCTRDIRGNWKYGGWFDPWGQGTLVTVSS

SEQ ID NO: 307 - DNA, 24 bp, synthetic construct
  misc_feature 1..24: note = Synthetic
  source 1..24: mol_type = other DNA
ggattcacctttgatgattatgcc

SEQ ID NO: 308 - protein, 8 aa, synthetic construct
  REGION 1..8: note = Synthetic
GFTFDDYA

SEQ ID NO: 309 - DNA, 24 bp, synthetic construct
  misc_feature 1..24: note = Synthetic
  source 1..24: mol_type = other DNA
attagttggactggtggtactata

SEQ ID NO: 310 - protein, 8 aa, synthetic construct
  REGION 1..8: note = Synthetic
ISWTGGTI

SEQ ID NO: 311 - DNA, 48 bp, synthetic construct
  misc_feature 1..48: note = Synthetic
  source 1..48: mol_type = other DNA
acaagagatatccgggggaactggaagtacggaggctggttcgacccc

SEQ ID NO: 312 - protein, 16 aa, synthetic construct
  REGION 1..16: note = Synthetic
TRDIRGNWKYGGWFDP

SEQ ID NO: 313 - DNA, 360 bp, synthetic construct
  misc_feature 1..360: note = Synthetic
  source 1..360: mol_type = other DNA
caggtgcagctggtgcagtctgggactgaggtgaagaagcctggggcctcagtgaaggtctcctgcaaggcttctggatacaccttcaccgcctactatatgcactgggtgcgacaggcccctggtcaaggacttgactggatgggatggatcagccctaacagtggtttcacaaactatgcacagaagtttcagggcagggtcaccatgaccagggacacgtccatcaacacattttatatggagctgagtggactgagatctgacgacacggccgtatattactgtgcgcgagagggttctactcaccacaattctttcgacccctggggccagggaaccctggtcaccgtctcctca

SEQ ID NO: 314 - protein, 120 aa, synthetic construct
  REGION 1..120: note = Synthetic
QVQLVQSGTEVKKPGASVKVSCKASGYTFTAYYMHWVRQAPGQGLDWMGWISPNSGFTNYAQKFQGRVTMTRDTSINTFYMELSGLRSDDTAVYYCAREGSTHHNSFDPWGQGTLVTVSS

SEQ ID NO: 315 - DNA, 24 bp, synthetic construct
  misc_feature 1..24: note = Synthetic
  source 1..24: mol_type = other DNA
ggatacaccttcaccgcctactat

SEQ ID NO: 316 - protein, 8 aa, synthetic construct
  REGION 1..8: note = Synthetic
GYTFTAYY

SEQ ID NO: 317 - DNA, 24 bp, synthetic construct
  misc_feature 1..24: note = Synthetic
  source 1..24: mol_type = other DNA
atcagccctaacagtggtttcaca

SEQ ID NO: 318 - protein, 8 aa, synthetic construct
  REGION 1..8: note = Synthetic
ISPNSGFT

SEQ ID NO: 319 - DNA, 39 bp, synthetic construct
  misc_feature 1..39: note = Synthetic
  source 1..39: mol_type = other DNA
gcgcgagagggttctactcaccacaattctttcgacccc

SEQ ID NO: 320 - protein, 13 aa, synthetic construct
  REGION 1..13: note = Synthetic
AREGSTHHNSFDP

SEQ ID NO: 321 - DNA, 342 bp, synthetic construct
  misc_feature 1..342: note = Synthetic
  source 1..342: mol_type = other DNA
gaggtgcagctggtggagtctggaggaggcttggtccaaccgggggggtccctgaggctctcctgtgcagcctctgggttcaccgtcggtactaacttcatgaattgggtccgccaggctccagggaaggggctggagtgggtctcagcgatttatagcggtggtaccgctaactacgcagactccgtgaagggccgattcaccatttccagagacacttccaggaacacgctgtatcttcaaatgaacagcctgagaactgaggacacggccgtttattattgtgcgagaggggggggtatggacgtctggggccaagggaccacggtcaccgtctcctca

SEQ ID NO: 322 - protein, 114 aa, synthetic construct
  REGION 1..114: note = Synthetic
EVQLVESGGGLVQPGGSLRLSCAASGFTVGTNFMNWVRQAPGKGLEWVSAIYSGGTANYADSVKGRFTISRDTSRNTLYLQMNSLRTEDTAVYYCARGGGMDVWGQGTTVTVSS

SEQ ID NO: 323 - DNA, 24 bp, synthetic construct
  misc_feature 1..24: note = Synthetic
  source 1..24: mol_type = other DNA
gggttcaccgtcggtactaacttc

SEQ ID NO: 324 - protein, 8 aa, synthetic construct
  REGION 1..8: note = Synthetic
GFTVGTNF

SEQ ID NO: 325 - DNA, 21 bp, synthetic construct
  misc_feature 1..21: note = Synthetic
  source 1..21: mol_type = other DNA
atttatagcggtggtaccgct

SEQ ID NO: 326 - protein, 7 aa, synthetic construct
  REGION 1..7: note = Synthetic
IYSGGTA

SEQ ID NO: 327 - DNA, 24 bp, synthetic construct
  misc_feature 1..24: note = Synthetic
  source 1..24: mol_type = other DNA
gcgagaggggggggtatggacgtc

SEQ ID NO: 328 - protein, 8 aa, synthetic construct
  REGION 1..8: note = Synthetic
ARGGGMDV

SEQ ID NO: 329 - DNA, 354 bp, synthetic construct
  misc_feature 1..354: note = Synthetic
  source 1..354: mol_type = other DNA
caggtccagctggtgcagtctggggctgaggtgaagaagcctgggtcctcggtgaaggtctcctgcaaggcttctggaggcaccttcaacacctatgttctcagctgggtgcgacaggcccctggacaagggcttgagtggatgggagagatcatccctatcttaggtgcagcaaactacgcacagaacttccagggcagagtcacttttaccacggacgaatccacgaatacagcctacatggacctgagcagcctaagatctgaggacacggccgtgtattactgtgcgagagatcggacctccggggggttcgacccctggggccagggaaccctggtcactgtctcctca

SEQ ID NO: 330 - protein, 118 aa, synthetic construct
  REGION 1..118: note = Synthetic
QVQLVQSGAEVKKPGSSVKVSCKASGGTFNTYVLSWVRQAPGQGLEWMGEIIPILGAANYAQNFQGRVTFTTDESTNTAYMDLSSLRSEDTAVYYCARDRTSGGFDPWGQGTLVTVSS

SEQ ID NO: 331 - DNA, 24 bp, synthetic construct
  misc_feature 1..24: note = Synthetic
  source 1..24: mol_type = other DNA
ggaggcaccttcaacacctatgtt

SEQ ID NO: 332 - protein, 8 aa, synthetic construct
  REGION 1..8: note = Synthetic
GGTFNTYV

SEQ ID NO: 333 - DNA, 24 bp, synthetic construct
  misc_feature 1..24: note = Synthetic
  source 1..24: mol_type = other DNA
atcatccctatcttaggtgcagca

SEQ ID NO: 334 - protein, 8 aa, synthetic construct
  REGION 1..8: note = Synthetic
IIPILGAA

SEQ ID NO: 335 - DNA, 33 bp, synthetic construct
  misc_feature 1..33: note = Synthetic
  source 1..33: mol_type = other DNA
gcgagagatcggacctccggggggttcgacccc

SEQ ID NO: 336 - protein, 11 aa, synthetic construct
  REGION 1..11: note = Synthetic
ARDRTSGGFDP

SEQ ID NO: 337 - DNA, 357 bp, synthetic construct
  misc_feature 1..357: note = Synthetic
  source 1..357: mol_type = other DNA
caggttcagctggtgcagtctggagctgaggtggagaagcctggggcctcagtgaaggtctcctgcaaggcttctggttacatctttacccactatggtatcagctgggtgcgacaggcccctggacaaggacttgagtgggtgggctggatcagcccttacaatggttacacagactatgcacagaaactccagggcagagtcaccttgaccacagacacatccacgaccacagcctacatggagctgaggaacctgagatctgacgacacggccatgtattactgttcgagagggaggggcccttactggtccttcgatctctggggccgtggcaccctggtcaccgtctcctca

SEQ ID NO: 338 - protein, 119 aa, synthetic construct
  REGION 1..119: note = Synthetic
QVQLVQSGAEVEKPGASVKVSCKASGYIFTHYGISWVRQAPGQGLEWVGWISPYNGYTDYAQKLQGRVTLTTDTSTTTAYMELRNLRSDDTAMYYCSRGRGPYWSFDLWGRGTLVTVSS

SEQ ID NO: 339 - DNA, 24 bp, synthetic construct
  misc_feature 1..24: note = Synthetic
  source 1..24: mol_type = other DNA
ggttacatctttacccactatggt

SEQ ID NO: 340 - protein, 8 aa, synthetic construct
  REGION 1..8: note = Synthetic
GYIFTHYG

SEQ ID NO: 341 - DNA, 24 bp, synthetic construct
  misc_feature 1..24: note = Synthetic
  source 1..24: mol_type = other DNA
atcagcccttacaatggttacaca

SEQ ID NO: 342 - protein, 8 aa, synthetic construct
  REGION 1..8: note = Synthetic
ISPYNGYT

SEQ ID NO: 343 - DNA, 36 bp, synthetic construct
  misc_feature 1..36: note = Synthetic
  source 1..36: mol_type = other DNA
tcgagagggaggggcccttactggtccttcgatctc

SEQ ID NO: 344 - protein, 12 aa, synthetic construct
  REGION 1..12: note = Synthetic
SRGRGPYWSFDL

SEQ ID NO: 345 - protein, 249 aa, synthetic construct
  REGION 1..249: note = hPD-L1-MMH
  REGION 1..221: note = 1-221: aa 19-239 of NP_054862.1
  REGION 222..249: note = 222-249: myc-myc-hexahistidine tag
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTEQKLISEEDLGGEQKLISEEDLHHHHHH

SEQ ID NO: 346 - protein, 249 aa, synthetic construct
  REGION 1..249: note = MfPD-L1-MMH
  REGION 1..221: note = 1-221:MfPD-L1
  REGION 222..249: note = 222-249: myc-myc-hexahistidine tag
FTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERTEQKLISEEDLGGEQKLISEEDLHHHHHH

SEQ ID NO: 347 - protein, 448 aa, synthetic construct
  REGION 1..448: note = hPD-L1-hFc
  REGION 1..221: note = 1-221: aa 19-239 of NP_054862.1
  REGION 222..448: note = 222-448: aa 104-330 of P01857
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 348 - protein, 454 aa, synthetic construct
  REGION 1..454: note = hPD-L1-mFc
  REGION 1..221: note = 1-221: aa 19-239 of NP_054862.1
  REGION 222..454: note = 222-454: aa 98-330 of P01863
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 349 - protein, 379 aa, synthetic construct
  REGION 1..379: note = hPD-1-mFc
  REGION 1..146: note = 1-146: aa 25-170 of NP_005009.2 with C93S
  REGION 147..379: note = 147-379: aa 98-330 of P01863
LDSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQFQTLVEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 350 - protein, 373 aa, synthetic construct
  REGION 1..373: note = hPD-1-hFc
  REGION 1..146: note = 1-146: aa 25-170 of NP_005009.2 with C93S
  REGION 141..373: note = 147-373: aa 104-330 of P01857
LDSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQFQTLVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 290 aa, synthetic construct
  REGION 1..290: note = hPD-L1 NP_054862.1
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 352 - protein, 288 aa, synthetic construct
  REGION 1..288: note = hPD-1 NP_005009.2
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 353 - protein, 454 aa, synthetic construct
  REGION 1..454: note = MfPD-L1-mFc aa1-221: Mf PD-L1 (aa 19-239 of XP_005581836.1) aa222-454: mFc tag (aa 98-330 of P01863)
FTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERTEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

(19) United States
(12) Patent Application Publication
(10) Pub. No.: US 2025/0092139 A1
(43) Pub. Date: Mar. 20, 2025

(54) HUMAN ANTIBODIES TO PD-L1

(71) Applicant: Regeneron Pharmaceuticals, Inc., Tarrytown, NY (US)

(72) Inventors: Nicholas J. Papadopoulos, LaGrangeville, NY (US); Andrew J. Murphy, Croton-on-Hudson, NY (US); Gavin Thurston, Briarcliff Manor, NY (US); Ella Ioffe, Bronx, NY (US); Elena Burova, Mount Kisco, NY (US)

(21) Appl. No.: 18/442,255

(22) Filed: Feb. 15, 2024

Related U.S. Application Data
(63) Continuation of application No. 17/399,829, filed on Aug. 11, 2021, now abandoned.
Application No. 17/399,829 is a continuation of application No. 15/905,653, filed on Feb. 26, 2018, now Pat. No. 11,117,970.
Application No. 15/905,653 is a continuation of application No. 14/603,808, filed on Jan. 23, 2015, now Pat. No. 9,938,345.
(60) Provisional application No. 61/930,582, filed on Jan. 23, 2014.
(60) Provisional application No. 62/089,549, filed on Dec. 9, 2014.

(51) Int. Cl. C07K 16/28; A61K 39/00; A61K 39/395; A61K 45/06; C07K 16/22; C07K 16/30

(52) U.S. Cl. CPC C07K 16/2827; A61K 39/3955; A61K 39/39558; A61K 45/06; C07K 16/22; C07K 16/30; A61K 2039/505; A61K 2039/507; C07K 2317/21; C07K 2317/565; C07K 2317/75; C07K 2317/76; C07K 2317/92

(57) ABSTRACT

The present invention provides antibodies that bind to the T-cell co-inhibitor ligand programmed death-ligand 1 (PD-L1) protein, and methods of use. In various embodiments of the invention, the antibodies are fully human antibodies that bind to PD-L1. In certain embodiments, the present invention provides multi-specific antigen-binding molecules comprising a first binding specificity that binds to PD-L1 and a second binding specificity that binds to a tumor cell antigen, an infected cell-specific antigen, or a T-cell co-inhibitor. In some embodiments, the antibodies of the invention are useful for inhibiting or neutralizing PD-L1 activity, thus providing a means of treating a disease or disorder such as cancer or viral infection.

### CROSS-REFERENCE TO RELATED APPLICATIONS

This application is a continuation of U.S. patent application Ser. No. 17/399,829 filed Aug. 11, 2021, which is a continuation of U.S. patent application Ser. No. 15/905,653 filed Feb. 26, 2018 (now U.S. Pat. No. 11,117,970), which is a continuation of U.S. patent application Ser. No. 14/603,808 filed Jan. 23, 2015 (now U.S. Pat. No. 9,938,345), which claims the benefit of priority under 35 U.S.C. § 119 (e) of U.S. Provisional Application No. 61/930,582 filed Jan. 23, 2014 and U.S. Provisional Application No. 62/089,549 filed Dec. 9, 2014, the disclosures of all of which are hereby incorporated by reference in their entireties.

### SEQUENCE LISTING

The sequence listing of the present application is submitted electronically as a computer readable sequence listing in XML format with a file name “SeqListA0029.xml,” a creation date of Feb. 13, 2024, and a size of 432,258 bytes. This sequence listing submitted is part of the specification and is hereby incorporated by reference in its entirety.

### FIELD OF THE INVENTION

The present invention is related to human antibodies and antigen-binding fragments of human antibodies that specifically bind to the immunomodulatory receptor ligand programmed death-ligand 1 (PD-L1), and therapeutic and diagnostic methods of using those antibodies.

STATEMENT OF RELATED ART

Programmed death-ligand 1 (PD-L1) (also called B7-H1 or CD274) is a 290 amino acid protein receptor ligand expressed widely on both lymphoid and non-lymphoid tissues such as CD4 and CD8 T-cells, macrophage lineage cells, peripheral tissues as well as on tumor cells, and virally-infected cells (Dong et al 1999, Nature Med.). PD-L1 binds to receptors PD-1 and B7-1 which belong to the CD28/CTLA-4 (cytotoxic T lymphocyte antigen)/ICOS (inducible co-stimulator) family of T-cell co-inhibitory receptors (Chen et al 2013, Nature Rev. Immunol. 13:227-242) and attenuates the immune response by inhibiting T-cell activation. PD-L1 binding to PD-1 or B7-1 results in decreased T-cell proliferation and cytokine secretion, compromising humoral and cellular immune responses in diseases such as cancer, and viral infection.

The expression of PD-L1 on tumor cells and virally-infected cells is exploited by tumors and chronic viral infections to evade immune response. PD-L1 is expressed on a wide variety of tumors and studies on animal models have shown that PD-L1 on tumors inhibits T-cell activation and lysis of tumor cells and may lead to increased death of tumor-specific T-cells. In chronic viral infections, PD-L1 expressed on virally-infected cells binds to PD-1 on virus-specific T-cells and these T-cells become “exhausted” with loss of effector functions and proliferative capacity (Freeman 2008, PNAS 105:10275-10276). The PD-1: PD-L1 system also plays an important role in induced T-regulatory (Treg) cell development and in sustaining Treg function (Francisco et al 2010, Immunol. Rev. 236:219-242).

Since PD-L1 plays an important role in tumor immunity and infectious immunity, it is an ideal target for immunotherapy. Blocking PD-L1 with antagonists, including monoclonal antibodies, has been studied in treatments of cancer and chronic viral infections (Ribas 2012, NEJM 366:2517-2519; Freeman 2008, PNAS 105:10275-10276; Sheridan 2012, Nature Biotechnology 30:729-730).

Monoclonal antibodies to PD-L1 are known in the art and have been described, for example, in US Patent/Publication Nos. 7943742, 8383796, 8217149, 20090055944, 20120003056, 20130034559, 20130045200, 20130045201, 20130045202, and in WO2007005874, WO2011066389, WO2010077634, EP1907424, and EP1899379.

### BRIEF SUMMARY OF THE INVENTION

The present invention provides antibodies and antigen-binding fragments thereof that bind PD-L1. The antibodies of the present invention are useful, inter alia, for targeting cells expressing PD-L1 such as cancer cells or virally-infected cells, and for modulating PD-L1 activity. In certain embodiments, the antibodies of the invention are useful for inhibiting or neutralizing PD-L1 activity and for stimulating T cell activation, e.g., under circumstances where T cell-mediated killing is beneficial or desirable. The anti-PD-L1 antibodies of the invention, or antigen-binding portions thereof, may be included as part of a multi-specific antigen-binding molecule, for example, to modulate the immune response and/or to target the antibodies to a specific cell type, such as a tumor cell, or a virally infected cell. The antibodies are useful in treating a disease or disorder such as cancer and viral infection.

The antibodies of the invention can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al., 2000, J. Immunol. 164:1925-1933). In certain embodiments, the antibodies may be bispecific.

In a first aspect, the present invention provides isolated monoclonal antibodies or antigen-binding fragments thereof that bind specifically to PD-L1. Exemplary anti-PD-L1 antibodies of the present invention are listed in Tables 1 and 2 herein. Table 1 sets forth the amino acid sequence identifiers of the heavy chain variable regions (HCVRs), light chain variable regions (LCVRs), heavy chain complementarity determining regions (HCDR1, HCDR2 and HCDR3), and light chain complementarity determining regions (LCDR1, LCDR2 and LCDR3) of the exemplary anti-PD-L1 antibodies. Table 2 sets forth the nucleic acid sequence identifiers of the HCVRs, LCVRs, HCDR1, HCDR2 HCDR3, LCDR1, LCDR2 and LCDR3 of the exemplary anti-PD-L1 antibodies.

The present invention provides antibodies, or antigen-binding fragments thereof, comprising an HCVR comprising an amino acid sequence selected from any of the HCVR amino acid sequences listed in Table 1, or a substantially similar sequence thereof having at least 90%, at least 95%, at least 98% or at least 99% sequence identity thereto.

The present invention also provides antibodies, or antigen-binding fragments thereof, comprising an LCVR comprising an amino acid sequence selected from any of the LCVR amino acid sequences listed in Table 1, or a substantially similar sequence thereof having at least 90%, at least 95%, at least 98% or at least 99% sequence identity thereto.

The present invention also provides antibodies, or antigen-binding fragments thereof, comprising an HCVR and an LCVR amino acid sequence pair (HCVR/LCVR) comprising any of the HCVR amino acid sequences listed in Table 1 paired with any of the LCVR amino acid sequences listed in Table 1. According to certain embodiments, the present invention provides antibodies, or antigen-binding fragments thereof, comprising an HCVR/LCVR amino acid sequence pair contained within any of the exemplary anti-PD-L1 antibodies listed in Table 1. In certain embodiments, the HCVR/LCVR amino acid sequence pair is selected from the group consisting of SEQ ID NOs: 2/10, 18/26, 34/42, 50/58, 66/74, 82/90, 98/106, 114/122, 130/138, 146/154, 162/170, 178/170, 186/194, 202/210, 218/226, 234/242, 250/258, 266/274, 282/274, 290/274, 298/274, 306/274, 314/274, 322/274, 330/274, and 338/274. In certain embodiments, the HCVR/LCVR amino acid sequence pair is selected from one of SEQ ID NOs: 82/90 (e.g., H2M8314N), 162/170 (e.g., H2M8718N), 306/274 (e.g., H1H9364P2), and 314/274 (e.g., H1H9373P2). In certain other embodiments, the HCVR/LCVR amino acid sequence pair is selected from one of SEQ ID NOs: 98/106 (e.g., H2M8316N), 146/154 (e.g., H2M8323N), 290/274 (e.g., H1H9351P2), and 330/274 (e.g., H1H9387P2).

The present invention also provides antibodies, or antigen-binding fragments thereof, comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR1 (LCDR1) comprising an amino acid sequence selected from any of the LCDR1 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR2 (LCDR2) comprising an amino acid sequence selected from any of the LCDR2 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR3 (LCDR3) comprising an amino acid sequence selected from any of the LCDR3 amino acid sequences listed in Table 1 or a substantially similar sequence thereof having at least 90%, at least 95%, at least 98% or at least 99% sequence identity.

The present invention also provides antibodies, or antigen-binding fragments thereof, comprising an HCDR3 and an LCDR3 amino acid sequence pair (HCDR3/LCDR3) comprising any of the HCDR3 amino acid sequences listed in Table 1 paired with any of the LCDR3 amino acid sequences listed in Table 1. According to certain embodiments, the present invention provides antibodies, or antigen-binding fragments thereof, comprising an HCDR3/LCDR3 amino acid sequence pair contained within any of the exemplary anti-PD-L1 antibodies listed in Table 1. In certain embodiments, the HCDR3/LCDR3 amino acid sequence pair is selected from the group consisting of SEQ ID NOs: 88/96 (e.g., H2M8314N), 168/176 (e.g., H2M8718N), 312/280 (e.g., H1H9364P2), and 320/280 (e.g., H1H9373P2). In certain other embodiments, the HCDR3/LCDR3 amino acid sequence pair is selected from the group consisting of SEQ ID NOs: 104/112 (e.g., H2M8316N), 152/160 (e.g., H2M8323N), 296/280 (e.g., H1H9351P2), and 336/280 (e.g., H1H9387P2).

The present invention also provides antibodies, or antigen-binding fragments thereof, comprising a set of six CDRs (i.e., HCDR1-HCDR2-HCDR3-LCDR1-LCDR2-LCDR3) contained within any of the exemplary anti-PD-L1 antibodies listed in Table 1. In certain embodiments, the HCDR1-HCDR2-HCDR3-LCDR1-LCDR2-LCDR3 amino acid sequence set is selected from the group consisting of SEQ ID NOs: 84-86-88-92-94-96 (e.g., H2M8314N); 164-166-168-172-174-176 (e.g., H2M8718N); 308-310-312-276-278-280 (e.g., H1H9364P2); and 316-318-320-276-278-280 (e.g., H1H9373P2). In certain other embodiments, the HCDR1-HCDR2-HCDR3-LCDR1-LCDR2-LCDR3 amino acid sequence set is selected from the group consisting of SEQ ID NOs: 100-102-104-108-110-112 (e.g., H2M8316N); 148-150-152-156-158-160 (e.g., H2M8323N); 292-294-296-276-278-280 (e.g., H1H9351P2); and 332-334-336-276-278-280 (e.g., H1H9387P2).

In a related embodiment, the present invention provides antibodies, or antigen-binding fragments thereof, comprising a set of six CDRs (i.e., HCDR1-HCDR2-HCDR3-LCDR1-LCDR2-LCDR3) contained within an HCVR/LCVR amino acid sequence pair as defined by any of the exemplary anti-PD-L1 antibodies listed in Table 1. For example, the present invention includes antibodies, or antigen-binding fragments thereof, comprising the HCDR1-HCDR2-HCDR3-LCDR1-LCDR2-LCDR3 amino acid sequences set contained within an HCVR/LCVR amino acid sequence pair selected from the group consisting of SEQ ID NOs: 82/90 (e.g., H2M8314N), 98/106 (e.g., H2M8316N), 146/154 (e.g., H2M8323N), 162/170 (e.g., H2M8718N), 290/274 (e.g., H1H9351P2), 306/274 (e.g., H1H9364P2), 314/274 (e.g., H1H9373P2) and 330/274 (e.g., H1H9387P2). Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

The present invention includes anti-PD-L1 antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

The present invention also provides for antibodies and antigen-binding fragments thereof that compete for specific binding to PD-L1 with an antibody or antigen-binding fragment thereof comprising the CDRs of a HCVR and the CDRs of a LCVR, wherein the HCVR and LCVR each has an amino acid sequence selected from the HCVR and LCVR sequences listed in Table 1.

The present invention also provides isolated antibodies and antigen-binding fragments thereof that block PD-L1 binding to PD-1 or to B7-1. In some embodiments, the antibody or antigen-binding fragment thereof that blocks PD-L1 binding to PD-1 or to B7-1 may bind to the same epitope on PD-L1 as PD-1/B7-1 or may bind to a different epitope on PD-L1 as PD-1/B7-1. In certain embodiments, the antibodies of the invention that block PD-L1 binding to PD-1 or to B7-1 comprise the CDRs of an HCVR having an amino acid sequence selected from the group consisting of HCVR sequences listed in Table 1; and the CDRs of a LCVR having an amino acid sequence selected from the group consisting of LCVR sequences listed in Table 1.

In alternate embodiments, the present invention provides antibodies and antigen-binding fragments thereof that do not block PD-L1 binding to PD-1 or to B7-1. In certain embodiments, the present invention provides isolated antibodies or antigen-binding fragments thereof that bind PD-L1, wherein the antibodies or antigen-binding fragments thereof enhance PD-L1 binding to PD-1 or to B7-1. In some embodiments, the isolated antibodies or antigen-binding fragments thereof that enhance PD-L1 binding to PD-1/B7-1 comprise the CDRs of a HCVR, wherein the HCVR has an amino acid sequence selected from the group consisting of SEQ ID NOs: 18, 66, 114, 130, 202, 218, 266, 282, 298, 322 and 338; and the CDRs of a LCVR, wherein the LCVR has an amino acid sequence selected from the group consisting of SEQ ID NOs: 26, 74, 122, 138, 210, 226, and 274. In some embodiments, the isolated antibodies or antigen-binding fragments thereof comprise an HCVR/LCVR amino acid sequence pair selected from the group consisting of SEQ ID NOs: 18/26 (e.g., H2M8307N), 66/74 (e.g., H2M8312N), 114/122 (e.g., H2M8317N), 130/138 (e.g., H2M8321N), 202/210 (e.g., H1H9323P), 218/226 (e.g., H1H9327P), 266/274 (e.g., H1H9344P2), 282/274 (e.g., H1H9345P2), 298/274 (e.g., H1H9354P2), 322/274 (e.g., H1H9382P2), and 338/274 (e.g., H1H9396P2).

The present invention also provides antibodies and antigen-binding fragments thereof that bind specifically to PD-L1 from human or other species. In certain embodiments, the antibodies may bind to human PD-L1 and/or to cynomolgus PD-L1.

The present invention also provides antibodies and antigen-binding fragments thereof that cross-compete for binding to PD-L1 with a reference antibody or antigen-binding fragment thereof comprising the CDRs of a HCVR and the CDRs of a LCVR, wherein the HCVR and LCVR each has an amino acid sequence selected from the HCVR and LCVR sequences listed in Table 1.

In one embodiment, the invention provides an isolated antibody or antigen-binding fragment that has one or more of the following characteristics: (a) blocks the binding of PD-L1 to PD-1 or to B7-1; (b) binds specifically to human PD-L1 and/or cynomolgus PD-L1; (c) inhibits T-cell proliferation in a mixed lymphocyte reaction (MLR) assay; and (d) increases IL-2 and/or interferon-gamma secretion in a MLR assay.

In some embodiments, the antibody or antigen binding fragment thereof may bind specifically to PD-L1 in an agonist manner, i.e., it may enhance or stimulate PD-L1 binding and/or activity; in other embodiments, the antibody may bind specifically to PD-L1 in an antagonist manner, i.e., it may block PD-L1 from binding to its receptor.

In certain embodiments, the antibodies or antigen-binding fragments of the present invention are bispecific comprising a first binding specificity to PD-L1 and a second binding specificity for a second target epitope. The second target epitope may be another epitope on PD-L1 or on a different protein such as a T-cell co-inhibitor. In certain embodiments, the target epitope may be on a different cell including e.g., a different T-cell, a B-cell, a tumor cell, an autoimmune tissue cell or a virally infected cell.

In a second aspect, the present invention provides nucleic acid molecules encoding anti-PD-L1 antibodies or portions thereof. For example, the present invention provides nucleic acid molecules encoding any of the HCVR amino acid sequences listed in Table 1; in certain embodiments the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto.

The present invention also provides nucleic acid molecules encoding any of the LCVR amino acid sequences listed in Table 1; in certain embodiments the nucleic acid molecule comprises a polynucleotide sequence selected from any of the LCVR nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR1amino acid sequences listed in Table 1; in certain embodiments the nucleic acid molecule comprises a polynucleotide sequence selected from any of the HCDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR2amino acid sequences listed in Table 1; in certain embodiments the nucleic acid molecule comprises a polynucleotide sequence selected from any of the HCDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR3amino acid sequences listed in Table 1; in certain embodiments the nucleic acid molecule comprises a polynucleotide sequence selected from any of the HCDR3 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR1amino acid sequences listed in Table 1; in certain embodiments the nucleic acid molecule comprises a polynucleotide sequence selected from any of the LCDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR2amino acid sequences listed in Table 1; in certain embodiments the nucleic acid molecule comprises a polynucleotide sequence selected from any of the LCDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR3amino acid sequences listed in Table 1; in certain embodiments the nucleic acid molecule comprises a polynucleotide sequence selected from any of the LCDR3 nucleic acid sequences listed in Table 2, or a substantially similar sequence thereof having at least 90%, at least 95%, at least 98% or at least 99% sequence identity thereto.

The present invention also provides nucleic acid molecules encoding an HCVR, wherein the HCVR comprises a set of three CDRs (i.e., HCDR1-HCDR2-HCDR3), wherein the HCDR1-HCDR2-HCDR3 amino acid sequence set is as defined by any of the exemplary anti-PD-L1 antibodies listed in Table 1.

The present invention also provides nucleic acid molecules encoding an LCVR, wherein the LCVR comprises a set of three CDRs (i.e., LCDR1-LCDR2-LCDR3), wherein the LCDR1-LCDR2-LCDR3 amino acid sequence set is as defined by any of the exemplary anti-PD-L1 antibodies listed in Table 1.

The present invention also provides nucleic acid molecules encoding both an HCVR and an LCVR, wherein the HCVR comprises an amino acid sequence of any of the HCVR amino acid sequences listed in Table 1, and wherein the LCVR comprises an amino acid sequence of any of the LCVR amino acid sequences listed in Table 1. In certain embodiments, the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto, and a polynucleotide sequence selected from any of the LCVR nucleic acid sequences listed in Table 2, or a substantially similar sequence thereof having at least 90%, at least 95%, at least 98% or at least 99% sequence identity thereto. In certain embodiments according to this aspect of the invention, the nucleic acid molecule encodes an HCVR and LCVR, wherein the HCVR and LCVR are both derived from the same anti-PD-L1 antibody listed in Table 1.

In a related aspect, the present invention provides recombinant expression vectors capable of expressing a polypeptide comprising a heavy or light chain variable region of an anti-PD-L1 antibody. For example, the present invention includes recombinant expression vectors comprising any of the nucleic acid molecules mentioned above, i.e., nucleic acid molecules encoding any of the HCVR, LCVR, and/or CDR sequences as set forth in Table 1. Also included within the scope of the present invention are host cells into which such vectors have been introduced, as well as methods of producing the antibodies or portions thereof by culturing the host cells under conditions permitting production of the antibodies or antibody fragments, and recovering the antibodies and antibody fragments so produced.

In a third aspect, the present invention provides multi-specific antigen-binding molecules and antigen-binding fragments thereof comprising a first antigen-binding specificity that binds specifically to PD-L1 and a second antigen-binding specificity that binds specifically to an antigen selected from the group consisting of PD-L1, a tumor cell-specific antigen, an infected-cell-specific antigen, and a T-cell co-inhibitor. In certain embodiments, the first antigen-binding specificity may comprise three CDRs derived from a HCVR with an amino acid sequence selected from the HCVR sequences in Table 1 and three CDRs derived from a LCVR with an amino acid sequence selected from the LCVR sequences in Table 1. In one embodiment, the first antigen-binding specificity may comprise an extracellular domain of PD-1 or of B7-1, or a fragment thereof. The second antigen-binding specificity may target an antigen on the same cell as PD-L1 or on a different cell of the same tissue type or of a different tissue type. For example, the multi-specific antigen-binding molecule may bind to a T-cell wherein the first antigen-binding specificity may bind specifically to PD-L1 and the second antigen-binding specificity may bind to a T-cell co-inhibitor on the T-cell. Alternatively, in another embodiment, the first antigen-binding specificity binds specifically to PD-L1 on a T-cell and the second antigen-binding specificity is targeted to an antigen/receptor on a B-cell or a macrophage or antigen-presenting cell. In certain embodiments, the second antigen-binding specificity is directed to an antigen on a tumor cell, or on a cell infected with a virus. In one embodiment, the first antigen-binding specificity comprises an extracellular domain of PD-1 and the second antigen-binding specificity binds to a different epitope on PD-L1. In certain embodiments, the first antigen-binding specificity binds to PD-L1 with a lower affinity, for example, with a K_{D} more than 10⁻⁸ M, more than 10⁻⁷ M, more than 10⁻⁶ M, or more than 10⁻⁵ M.

In a fourth aspect, the invention provides a pharmaceutical composition comprising a recombinant human antibody or antigen-binding fragment thereof which specifically binds PD-L1 and a pharmaceutically acceptable carrier. In a related aspect, the invention features a composition which is a combination of an anti-PD-L1 antibody and a second therapeutic agent. In one embodiment, the second therapeutic agent is any agent that is advantageously combined with an anti-PD-L1 antibody. Exemplary agents that may be advantageously combined with an anti-PD-L1 antibody include, without limitation, other agents that bind and/or modulate PD-L1 signaling (including other antibodies or antigen-binding fragments thereof, etc.) and/or agents which do not directly bind PD-L1 but nonetheless modulate immune cell activation. Additional combination therapies and co-formulations involving the anti-PD-L1 antibodies and multi-specific antigen-binding molecules of the present invention are disclosed elsewhere herein.

In a fifth aspect, the invention provides methods to modulate the immune response in a subject, the method comprising administering a therapeutically effective amount of an anti-PD-L1 antibody or antigen-binding fragment thereof of the invention to the subject in need thereof. In certain embodiments, the invention provides methods to enhance the immune response in a subject, the methods comprising administering to the subject an effective amount of an antibody or fragment thereof of the invention that binds PD-L1 and blocks PD-L1 binding to PD-1 or to B7-1. In one embodiment, the invention provides a method to stimulate or enhance T-cell activation in a subject, the method comprising administering a blocking antibody or antigen-binding fragment thereof of the invention to the subject in need thereof. In one embodiment, the invention provides methods to inhibit a T-regulatory (Treg) cell in a subject, the methods comprising administering a blocking antibody or antigen-binding fragment thereof of the invention to the subject in need thereof. In certain embodiments, the subject in need thereof may suffer from a disease or disorder such as cancer or viral infection. In alternate embodiments, the present invention provides methods to inhibit T-cell activation, the methods comprising administering an activating antibody or antigen-binding fragment thereof of the invention to a subject in need thereof. In certain further embodiments, the subject in need thereof may suffer from an autoimmune disease.

In a sixth aspect, the invention provides therapeutic methods for treating a disease or disorder, for example, cancer or viral infection, in a subject using an anti-PD-L1 antibody or antigen-binding portion of an antibody of the invention, wherein the therapeutic methods comprise administering a therapeutically effective amount of a pharmaceutical composition comprising an antibody or fragment of an antibody of the invention to the subject in need thereof. The disorder treated is any disease or condition which is improved, ameliorated, inhibited or prevented by stimulation or inhibition of PD-L1 binding or activity. In certain embodiments, the antibody or antigen-binding fragment thereof the invention is administered in combination with a second therapeutic agent to the subject in need thereof. The second therapeutic agent may be selected from the group consisting of an antibody to a T-cell co-inhibitor, an antibody to a tumor cell antigen, an antibody to a T-cell receptor, an antibody to a Fc receptor, an antibody to an epitope on a virally infected cell, an antibody to PD-1, a cytotoxic agent, an anti-cancer drug, an anti-viral drug, an anti-inflammatory drug (e.g., corticosteroids), a VEGF antagonist, and any other drug or therapy known in the art. In certain embodiments, the second therapeutic agent may be an agent that helps to counteract or reduce any possible side effect(s) associated with an antibody or antigen-binding fragment thereof of the invention, if such side effect(s) should occur.

In certain embodiments, the present invention provides methods for suppressing tumor growth. In certain embodiments, the present invention provides methods to enhance survival of cancer patients. Examples of cancer include, but are not limited to, primary and/or recurrent cancer, including brain cancer (e.g., glioblastoma multiforme), lung cancer (e.g., non-small cell lung cancer), squamous cell carcinoma of head and neck, renal cell carcinoma, melanoma, multiple myeloma, prostate cancer, and colon cancer. The methods comprise administering a pharmaceutical composition comprising a therapeutically effective amount of an anti-PD-L1 antibody of the present invention in combination with a second therapeutic agent selected from the group consisting of a vascular endothelial growth factor (VEGF) antagonist (e.g., aflibercept, bevacizumab), an angiopoietin-2 (Ang2) inhibitor (e.g., an anti-Ang2 antibody such as nesvacumab), a lymphocyte activation gene 3 (LAG-3) inhibitor, a cytotoxic T-lymphocyte antigen 4 (CTLA-4) inhibitor (e.g., ipilimumab), a CCR4 inhibitor (e.g., mogamulizumab), a chemotherapeutic agent, and radiation therapy. Additional examples of additional therapies/therapeutic agents that can be used in combination with an anti-PD-L1 antibody of the invention for use in treating cancer are described elsewhere herein.

The antibody or fragment thereof may be administered subcutaneously, intravenously, intradermally, intraperitoneally, orally, intramuscularly, or intracranially. The antibody or fragment thereof may be administered at a dose of about 0.1 mg/kg of body weight to about 100 mg/kg of body weight of the subject.

The present invention also includes use of an anti-PD-L1 antibody or antigen-binding fragment thereof of the invention in the manufacture of a medicament for the treatment of a disease or disorder (e.g., cancer, and chronic viral infection) that would benefit from the blockade or enhancement of PD-L1 binding and/or signaling.

Other embodiments will become apparent from a review of the ensuing detailed description.

### BRIEF DESCRIPTION OF THE FIGURES

FIG. 1 is a schematic of the luciferase-based PD-L1 bioassay described in Example 8 herein. Panel A: Inactive Jurkat cells; Panel B: Jurkat cells are activated by T-cell receptor (TCR) clustering through the CD3xCD20 bispecific antibody; Panel C: PD-1 activation attenuates response in activated Jurkat cells; Panel D: Blocking PD-L1 rescues the response in activated Jurkat cells.

FIG. 2 illustrates tumor growth and survival results for mice implanted with Colon-26 tumor cells at Day 0 and treated with the indicated combinations of molecules by injection at Days 3, 6, 10, 13 and 19 (“early-treatment tumor model”). The graph depicts tumor volume (in mm³) for the different experimental groups at various time points after implantation. Upward arrows along the X-axis indicate the timing of treatment injections. “mlgG2a” is IgG2 isotype control; “Fc” is human Fc control; “VEGF Trap” is aflibercept; “anti-PD-1” is anti-mouse PD-1 clone RPMI-14; “anti-PD-L1” is an anti-PD-L1 monoclonal antibody as described elsewhere herein.

FIG. 3 illustrates tumor growth and survival results for mice implanted with Colon-26 tumor cells at Day 0 and treated with the indicated combinations of molecules by injection at Days 3, 6, 10, 13 and 19 (“early-treatment tumor model”). The graph shows the tumor volume (in mm³) of individual mice in each experimental group at Day 28 after implantation. “mlgG2a” is IgG2 isotype control; “Fc” is human Fc control; “VEGF Trap” is aflibercept; “anti-PD-1” is anti-mouse PD-1 clone RPMI-14; “anti-PD-L1” is an anti-PD-L1 monoclonal antibody as described elsewhere herein.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, preferred methods and materials are now described. All publications mentioned herein are incorporated herein by reference in their entirety.

The term “PD-L1” refers to programmed death-ligand 1, also known as CD274 and B7H1. The amino acid sequence of full-length PD-L1 is provided in GenBank as accession number NP_054862.1 and is also referred to herein as SEQ ID NO: 351. The term “PD-L1” also includes protein variants of PD-L1 having the amino acid sequence of SEQ ID NOs: 345, 346, 347 or 348. The term “PD-L1” includes recombinant PD-L1 or a fragment thereof. The term also encompasses PD-L1 or a fragment thereof coupled to, for example, histidine tag, mouse or human Fc, or a signal sequence such as ROR1. For example, the term includes sequences exemplified by SEQ ID NOs: 347 or 348, comprising a mouse Fc (mlgG2a) or human Fc (hlgG1) at the C-terminal, coupled to amino acid residues 19-239 of full-length PD-L1 (SEQ ID NO: 351; NP_054862.1). Protein variants as exemplified by SEQ ID NO: 345 comprise a histidine tag at the C-terminal, coupled to amino acid residues 19-239 of NP_054862.1. Unless specified as being from a non-human species, the term “PD-L1” means human PD-L1.

PD-L1 is a 290 amino acid protein with extracellular IgV-like and IgC-like domains (amino acids 19-239 of full length PD-L1), a transmembrane domain and an intracellular domain of approximately 30 amino acids. PD-L1 is constitutively expressed on many cells such as antigen presenting cells (e.g., dendritic cells, macrophages, and B-cells) and on hematopoietic and non-hematopoietic cells (e.g., vascular endothelial cells, pancreatic islets, and sites of immune privilege). PD-L1 is also expressed on a wide variety of tumors, and virally-infected cells and is a component of the immunosuppressive milieu (Ribas 2012, NEJM 366:2517-2519). PD-L1 binds to one of two T-cell co-inhibitors PD-1 and B7-1.

The term “PD-1” refers to the programmed death-1 protein, a T-cell co-inhibitor, also known as CD279. The amino acid sequence of full-length PD-1 is provided in GenBank as accession number NP_005009.2 and is also referred to herein as SEQ ID NO: 352. The term also encompasses PD-1 or a fragment thereof coupled to, for example, histidine tag, mouse or human Fc, or a signal sequence such as ROR1. For example, the term includes sequences exemplified by SEQ ID NOs: 349 or 350, comprising a mouse Fc (mlgG2a) or human Fc (hlgG1) at the C-terminal, coupled to amino acid residues 25-170 of NP_005009.2 with a C93S change.

PD-1 is a member of the CD28/CTLA-4/ICOS family of T-cell co-inhibitors. PD-1 is a 288-amino acid protein with an extracellular N-terminal domain which is IgV-like, a transmembrane domain and an intracellular domain containing an immunoreceptor tyrosine-based inhibitory (ITIM) motif and an immunoreceptor tyrosine-based switch (ITSM) motif (Chattopadhyay et al 2009, Immunol. Rev.). The PD-1 receptor has two ligands, PD-L1 and PD-L2.

The term “B7-1” refers to the T-lymphocyte activation antigen, also known as costimulatory factor CD80. B7-1 is a 288 amino acid membrane receptor with an extracellular N-terminal domain which comprises IgV-like (aa 37-138) and IgC-like (aa 154-232) regions, a transmembrane domain (aa 243-263) and a C-terminal intracellular region (aa 263-288). The amino acid sequence of full-length B7-1 is provided in GenBank as accession number NP_005182.1.

As used herein, the term “T-cell co-inhibitor” refers to a ligand and/or receptor which modulates the immune response via T-cell activation or suppression. The term “T-cell co-inhibitor”, also known as T-cell co-signaling molecule, includes, but is not limited to, PD-1, lymphocyte activation gene 3 protein (LAG-3, also known as CD223), cytotoxic T-lymphocyte antigen-4 (CTLA-4), B and T lymphocyte attenuator (BTLA), CD-28, 2B4, LY108, T-cell immunoglobulin and mucin-3 (TIM3), T-cell immunoreceptor with immunoglobulin and ITIM (TIGIT; also known as VSIG9), leucocyte associated immunoglobulin-like receptor 1 (LAIR1; also known as CD305), inducible T-cell costimulator (ICOS; also known as CD278), B7-1 (CD80), and CD160.

The term “antibody”, as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is comprised of a heavy chain variable region (“HCVR” or “V_{H}”) and a heavy chain constant region (comprised of domains C_{H}1, C_{H}2 and C_{H}3). Each light chain is comprised of a light chain variable region (“LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments of the invention, the FRs of the antibody (or antigen binding fragment thereof) may be identical to the human germline sequences, or may be naturally or artificially modified. An amino acid consensus sequence may be defined based on a side-by-side analysis of two or more CDRs.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The fully human anti-PD-L1 monoclonal antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The present invention also includes fully human anti-PD-L1 monoclonal antibodies comprising variants of any of the HCVR, LCVR, and/or CDR amino acid sequences disclosed herein having one or more conservative substitutions. For example, the present invention includes anti-PD-L1 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “human antibody”, as used herein, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences. The term includes antibodies recombinantly produced in a non-human mammal, or in cells of a non-human mammal. The term is not intended to include antibodies isolated from or generated in a human subject.

The term “multi-specific antigen-binding molecules”, as used herein refers to bispecific, tri-specific or multi-specific antigen-binding molecules, and antigen-binding fragments thereof. Multi-specific antigen-binding molecules may be specific for different epitopes of one target polypeptide or may contain antigen-binding domains specific for epitopes of more than one target polypeptide. A multi-specific antigen-binding molecule can be a single multifunctional polypeptide, or it can be a multimeric complex of two or more polypeptides that are covalently or non-covalently associated with one another. The term “multi-specific antigen-binding molecules” includes antibodies of the present invention that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity. According to the present invention, the term “multi-specific antigen-binding molecules” also includes bi-specific, tri-specific or multi-specific antibodies or antigen-binding fragments thereof. In certain embodiments, an antibody of the present invention is functionally linked to another antibody or antigen-binding fragment thereof to produce a bispecific antibody with a second binding specificity. Bispecific and multi-specific antibodies of the present invention are described elsewhere herein.

The term “specifically binds,” or “binds specifically to”, or the like, means that an antibody or antigen-binding fragment thereof forms a complex with an antigen that is relatively stable under physiologic conditions. Specific binding can be characterized by an equilibrium dissociation constant of at least about 1×10⁻⁸ M or less (e.g., a smaller K_{D} denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, which bind specifically to PD-L1. Moreover, multi-specific antibodies that bind to one domain in PD-L1 and one or more additional antigens or a bi-specific that binds to two different regions of PD-L1 are nonetheless considered antibodies that “specifically bind”, as used herein.

The term “high affinity” antibody refers to those mAbs having a binding affinity to PD-L1, expressed as K_{D}, of at least 10⁻⁸ M; preferably 10⁻⁹ M; more preferably 10⁻¹⁰ M, even more preferably 10⁻¹¹ M, even more preferably 10⁻¹² M, as measured by surface plasmon resonance, e.g., BIACORE™ or solution-affinity ELISA.

By the term “slow off rate”, “Koff” or “kd” is meant an antibody that dissociates from PD-L1, with a rate constant of 1×10⁻³s⁻¹ or less, preferably 1×10⁻⁴s⁻¹ or less, as determined by surface plasmon resonance, e.g., BIACORE™.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to bind to PD-L1.

In specific embodiments, antibody or antibody fragments of the invention may be conjugated to a moiety such a ligand or a therapeutic moiety (“immunoconjugate”), such as an antibiotic, a second anti-PD-L1 antibody, or an antibody to another antigen such a tumor-specific antigen, a virally-infected cell antigen, or a T-cell co-inhibitor, or an immunotoxin, or any other therapeutic moiety useful for treating a disease or condition including e.g., cancer, chronic viral infection or autoimmune disease.

An “isolated antibody”, as used herein, is intended to refer to an antibody that is substantially free of other antibodies (Abs) having different antigenic specificities (e.g., an isolated antibody that specifically binds PD-L1, or a fragment thereof, is substantially free of Abs that specifically bind antigens other than PD-L1.

A “blocking antibody” or a “neutralizing antibody”, as used herein (or an “antibody that neutralizes PD-L1 activity” or an “antagonist antibody”), is intended to refer to an antibody whose binding to PD-L1 results in inhibition of at least one biological activity of PD-L1. For example, an antibody of the invention may prevent or block PD-L1 binding to PD-1 or to B7-1.

An “activating antibody” or an “enhancing antibody”, as used herein (or an “agonist antibody”), is intended to refer to an antibody whose binding to PD-L1 results in increasing or stimulating at least one biological activity of PD-L1. For example, an antibody of the invention may increase or enhance PD-L1 binding to PD-1 or to B7-1.

The term “surface plasmon resonance”, as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The term “K_{D}”, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding).

As used herein, the term “subject” refers to an animal, preferably a mammal, in need of amelioration, prevention and/or treatment of a disease or disorder such as chronic viral infection, cancer or autoimmune disease.

As used herein, “anti-cancer drug” means any agent useful to treat cancer including, but not limited to, cytotoxins and agents such as antimetabolites, alkylating agents, anthracyclines, antibiotics, antimitotic agents, procarbazine, hydroxyurea, asparaginase, corticosteroids, mytotane (O,P′-(DDD)), interferons and radioactive agents. As used herein, “a cytotoxin or cytotoxic agent” means any agent that is detrimental to cells. Examples include Taxol® (paclitaxel), cytochalasin B, gramicidin D, ethidium bromide, emetine, mitomycin, etoposide, tenoposide, vincristine, vinbiastine, coichicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

As used herein, the term “anti-viral drug” refers to any drug or therapy used to treat, prevent, or ameliorate a viral infection in a host subject. The term “anti-viral drug” includes, but is not limited to zidovudine, lamivudine, abacavir, ribavirin, lopinavir, efavirenz, cobicistat, tenofovir, rilpivirine, analgesics and corticosteroids. In the context of the present invention, the viral infections include long-term or chronic infections caused by viruses including, but not limited to, human immunodeficiency virus (HIV), hepatitis B virus (HBV), hepatitis C virus (HCV), human papilloma virus (HPV), lymphocytic choriomeningitis virus (LCMV), and simian immunodeficiency virus (SIV).

The antibodies and antigen-binding fragments of the present invention specifically bind to PD-L1 and modulate the interaction of PD-L1 with PD-1 or with B7-1. The anti-PD-L1 antibodies may bind to PD-L1 with high affinity or with low affinity. In certain embodiments, the antibodies of the present invention are blocking antibodies wherein the antibodies bind to PD-L1 and block the interaction of PD-L1 with PD-1 or with B7-1. In some embodiments, the blocking antibodies of the invention block the binding of PD-L1 to PD-1 or to B7-1 and/or stimulate or enhance T-cell activation. In some embodiments, the blocking antibodies are useful for stimulating or enhancing the immune response and/or for treating a subject suffering from cancer, or a chronic viral infection. The antibodies when administered to a subject in need thereof may reduce the chronic infection by a virus such as HIV, LCMV or HBV in the subject. They may be used to inhibit the growth of tumor cells in a subject. They may be used alone or as adjunct therapy with other therapeutic moieties or modalities known in the art for treating cancer, or viral infection. In certain embodiments, the anti-PD-L1 antibodies that bind to PD-L1 with a low affinity are used as multi-specific antigen-binding molecules wherein the first binding specificity binds to PD-L1 with a low affinity and the second binding specificity binds to an antigen selected from the group consisting of a different epitope of PD-L1, a T-cell co-inhibitor such as PD-1, a tumor specific antigen and an infected-cell-specific antigen.

In certain embodiments, the antibodies of the present invention are agonist antibodies, wherein the antibodies bind to PD-L1 and enhance the interaction of PD-L1 and PD-1/B7-1. In some embodiments, the activating antibodies enhance binding of PD-L1 to PD-1 or to B7-1 and/or inhibit or suppress T-cell activation. The activating antibodies of the present invention may be useful for inhibiting the immune response in a subject and/or for treating autoimmune disease.

In certain embodiments, the anti-PD-L1 antibodies are multi-specific antigen-binding molecules, wherein they comprise a first binding specificity to PD-L1 and a second binding specificity to an antigen selected from the group consisting of a different epitope of PD-L1, a T-cell co-inhibitor such as PD-1, a tumor specific antigen and an infected-cell-specific antigen. In certain embodiments, the first binding specificity binds to PD-L1 with low affinity, e.g., with a K_{D} of 10⁻⁸ M, 10⁻⁷ M or more.

Certain anti-PD-L1 antibodies of the present invention are able to bind to and neutralize the activity of PD-L1, as determined by in vitro or in vivo assays. The ability of the antibodies of the invention to bind to and neutralize the activity of PD-L1 may be measured using any standard method known to those skilled in the art, including binding assays, or activity assays, as described herein.

Non-limiting, exemplary in vitro assays for measuring binding activity are illustrated in Example 3, herein. In Example 3, the binding affinities and kinetic constants of human anti-PD-L1 antibodies for human PD-L1 and cynomolgus PD-L1 were determined by surface plasmon resonance and the measurements were conducted on a T200 Biacore instrument. In Examples 4 and 5, blocking assays were used to determine the ability of the anti-PD-L1 antibodies to block PD-L1-binding ability of PD-1 or to B7-1 in vitro. In Example 6, blocking assays were used to determine cross-competition between different anti-PD-L1 antibodies. Example 7 describes the binding of the antibodies to cells overexpressing PD-L1. In Example 8, a luciferase assay was used to determine the ability of anti-PD-L1 antibodies to antagonize PD-1/PD-L1 signaling in T-cells.

In certain embodiments, the antibodies of the present invention are able to enhance or stimulate T-cell activation in vitro and in a subject with cancer or in a subject infected with a virus such as LCMV. In certain embodiments, the antibodies of the present invention are used in combination with a second therapeutic agent, such as an antibody to a tumor-specific antigen or a T-cell co-inhibitor, to enhance the immune response and inhibit tumor growth in a subject. In certain embodiments, the agonist antibodies of the invention able to enhance PD-L1 binding to PD-1 or to B7-1 and may inhibit T-cell activation in vitro and/or in a subject with autoimmune disease.

The antibodies specific for PD-L1 may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface. In one embodiment, the label may be a radionuclide, a fluorescent dye or a MRI-detectable label. In certain embodiments, such labeled antibodies may be used in diagnostic assays including imaging assays.

### Antigen-Binding Fragments of Antibodies

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to specifically bind to PD-L1. An antibody fragment may include a Fab fragment, a F(ab′)₂ fragment, a Fv fragment, a dAb fragment, a fragment containing a CDR, or an isolated CDR. In certain embodiments, the term “antigen-binding fragment” refers to a polypeptide or fragment thereof of a multi-specific antigen-binding molecule. In such embodiments, the term “antigen-binding fragment” includes, e.g., an extracellular domain of PD-1 which binds specifically to PD-L1. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (V) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present invention using routine techniques available in the art.

### Preparation of Human Antibodies

Methods for generating human antibodies in transgenic mice are known in the art. Any such known methods can be used in the context of the present invention to make human antibodies that specifically bind to PD-L1.

An immunogen comprising any one of the following can be used to generate antibodies to PD-L1. In certain embodiments, the antibodies of the invention are obtained from mice immunized with a primary immunogen, such as a full length PD-L1 [See GenBank accession number NP_054862.1 (SEQ ID NO: 351)] or with a recombinant form of PD-L1 or modified human PD-L1 fragments (SEQ ID NOs: 345, 347 or 348) or with modified cynomolgus PD-L1 fragments (SEQ ID NO: 346), followed by immunization with a secondary immunogen, or with an immunogenically active fragment of PD-L1.

In certain embodiments, the immunogen may be a peptide from the N terminal or C terminal end of PD-L1. In one embodiment, the immunogen is the extracellular IgV-like and/or IgC-like domain of PD-L1. In certain embodiments of the invention, the immunogen is a fragment of PD-L1 that ranges from about amino acid residues 19-239 of SEQ ID NO: 351 (NP_054862.1).

In some embodiments, the immunogen may be a recombinant PD-L1 peptide expressed in E. coli or in any other eukaryotic or mammalian cells such as Chinese hamster ovary (CHO) cells.

In certain embodiments, antibodies that bind specifically to PD-L1 may be prepared using fragments of the above-noted regions, or peptides that extend beyond the designated regions by about 5 to about 20 amino acid residues from either, or both, the N or C terminal ends of the regions described herein. In certain embodiments, any combination of the above-noted regions or fragments thereof may be used in the preparation of PD-L1 specific antibodies.

Using VELOCIMMUNE® technology (see, for example, U.S. Pat. No. 6,596,541, Regeneron Pharmaceuticals, VELOCIMMUNER) or any other known method for generating monoclonal antibodies, high affinity chimeric antibodies to PD-L1 are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

### Bioequivalents

The anti-PD-L1 antibodies and antibody fragments of the present invention encompass proteins having amino acid sequences that vary from those of the described antibodies, but that retain the ability to bind PD-L1. Such variant antibodies and antibody fragments comprise one or more additions, deletions, or substitutions of amino acids when compared to parent sequence, but exhibit biological activity that is essentially equivalent to that of the described antibodies. Likewise, the antibody-encoding DNA sequences of the present invention encompass sequences that comprise one or more additions, deletions, or substitutions of nucleotides when compared to the disclosed sequence, but that encode an antibody or antibody fragment that is essentially bioequivalent to an antibody or antibody fragment of the invention.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, or potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include antibody variants comprising amino acid changes, which modify the glycosylation characteristics of the antibodies, e.g., mutations that eliminate or remove glycosylation.

### Anti-PD-L1 Antibodies Comprising Fc Variants

According to certain embodiments of the present invention, anti-PD-L1 antibodies are provided comprising an Fc domain comprising one or more mutations which enhance or diminish antibody binding to the FcRn receptor, e.g., at acidic pH as compared to neutral pH. For example, the present invention includes anti-PD-L1 antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present invention includes anti-PD-L1 antibodies comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 257I and 311I (e.g., P257I and Q311I); 257I and 434H (e.g., P257I and N434H); 376V and 434H (e.g., D376V and N434H); 307A, 380A and 434A (e.g., T307A, E380A and N434A); and 433K and 434F (e.g., H433K and N434F). In one embodiment, the present invention includes anti-PD-L1 antibodies comprising an Fc domain comprising a S108P mutation in the hinge region of IgG4 to promote dimer stabilization. All possible combinations of the foregoing Fc domain mutations, and other mutations within the antibody variable domains disclosed herein, are contemplated within the scope of the present invention.

The present invention also includes anti-PD-L1 antibodies comprising a chimeric heavy chain constant (C_{H}) region, wherein the chimeric C_{H} region comprises segments derived from the C_{H} regions of more than one immunoglobulin isotype. For example, the antibodies of the invention may comprise a chimeric C_{H} region comprising part or all of a C_{H}2 domain derived from a human IgG1, human IgG2 or human IgG4 molecule, combined with part or all of a C_{H}3 domain derived from a human IgG1, human IgG2 or human IgG4 molecule. According to certain embodiments, the antibodies of the invention comprise a chimeric C_{H} region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric C_{H} region as described herein may, in certain embodiments, exhibit modified Fc effector functions without adversely affecting the therapeutic or pharmacokinetic properties of the antibody. (See, e.g., U.S. Ser. No. 14/170,166, filed Jan. 31, 2014, the disclosure of which is hereby incorporated by reference in its entirety).

### Biological Characteristics of the Antibodies

In general, the antibodies of the present invention function by binding to PD-L1. The present invention includes anti-PD-L1 antibodies and antigen-binding fragments thereof that bind soluble monomeric or dimeric PD-L1 molecules with high affinity. For example, the present invention includes antibodies and antigen-binding fragments of antibodies that bind monomeric PD-L1 (e.g., at 25° C. or at 37° C.) with a K_{D} of less than about 318 pM as measured by surface plasmon resonance, e.g., using the assay format as defined in Example 3 herein. In certain embodiments, the antibodies or antigen-binding fragments thereof bind monomeric PD-L1 with a K_{D} of less than about 300 pM, less than about 250 pM, less than about 150 pM, less than about 100 pM, or less than about 50 pM, as measured by surface plasmon resonance, e.g., using the assay format as defined in Example 3 herein, or a substantially similar assay.

The present invention also includes antibodies and antigen-binding fragments thereof that bind dimeric PD-L1 (e.g., at 25° C. or at 37° C.) with a K_{D} of less than about 15 pM as measured by surface plasmon resonance, e.g., using the assay format as defined in Example 3 herein. In certain embodiments, the antibodies or antigen-binding fragments thereof bind dimeric PD-L1 with a K_{D} of less than about 12 pM, less than about 10 pM, less than about 8 pM, or less than about 5 pM, as measured by surface plasmon resonance, e.g., using the assay format as defined in Example 3 herein, or a substantially similar assay.

The present invention also includes antibodies or antigen-binding fragments thereof that bind cynomolgus (Macaca fascicularis) PD-L1 (e.g., at 25° C. or at 37° C.) with a K_{D} of less than about 28 nM as measured by surface plasmon resonance, e.g., using the assay format as defined in Example 3 herein. In certain embodiments, the antibodies or antigen-binding fragments thereof bind cynomolgus PD-L1 with a K_{D} of less than about 25 nM, less than about 20 nM, less than about 15 nM, less than about 10 nM, or less than about 5 nM, as measured by surface plasmon resonance, e.g., using the assay format as defined in Example 3 herein, or a substantially similar assay.

The present invention also includes antibodies and antigen-binding fragments thereof that bind PD-L1 with a dissociative half-life (t½) of greater than about 1 minute as measured by surface plasmon resonance at 25° C. or 37° C., e.g., using an assay format as defined in Example 3 herein, or a substantially similar assay. In certain embodiments, the antibodies or antigen-binding fragments of the present invention bind PD-L1 with a t½ of greater than about 5 minutes, greater than about 10 minutes, greater than about 30 minutes, greater than about 50 minutes, greater than about 60 minutes, greater than about 70 minutes, greater than about 80 minutes, greater than about 90 minutes, greater than about 100 minutes, greater than about 200 minutes, greater than about 300 minutes, greater than about 400 minutes, greater than about 500 minutes, greater than about 600 minutes, greater than about 700 minutes, or greater than about 800 minutes, as measured by surface plasmon resonance at 25° C. or 37° C., e.g., using an assay format as defined in Example 3 herein (e.g., mAb-capture or antigen-capture format), or a substantially similar assay.

The present invention also includes antibodies or antigen-binding fragments thereof that block PD-L1 binding to PD-1 with an IC₅₀ of less than about 770 pM as determined using a ELISA-based immunoassay assay, e.g., as shown in Example 4, or a substantially similar assay. The present invention also includes antibodies or antigen-binding fragments thereof that block PD-L1 binding to B7-1 with an IC₅₀ of less than about 10 nM as determined using a ELISA-based immunoassay assay, e.g., as shown in Example 4, or a substantially similar assay. The present invention also includes antibodies and antigen-binding fragments thereof that bind to PD-L1 and enhance the binding of PD-L1 to PD-1 or to B7-1.

In some embodiments, the antibodies of the present invention may bind to the extracellular domain of PD-L1 or to a fragment of the domain. In some embodiments, the antibodies of the present invention may bind to more than one domain (cross-reactive antibodies). In certain embodiments, the antibodies of the present invention may bind to an epitope located in the extracellular domain comprising amino acid residues 19-239 of NP_054862.1 (SEQ ID NO: 351). In some embodiments, the antibodies may bind to an epitope comprising one or more amino acids selected from the group consisting of amino acid residues 1-221 of SEQ ID NOs: 345-348, or 353.

In certain embodiments, the antibodies of the present invention may function by blocking or inhibiting the PD-1-binding or the B7-1-binding activity associated with PD-L1 by binding to any other region or fragment of the full length protein, the amino acid sequence of which is shown in SEQ ID NO: 351. In certain embodiments, the antibodies may attenuate or modulate the interaction between PD-L1 and PD-1/B7-1.

In certain embodiments, the antibodies of the present invention may be bi-specific antibodies. The bi-specific antibodies of the invention may bind one epitope in one domain and may also bind a second epitope in a different domain of PD-L1. In certain embodiments, the bi-specific antibodies of the invention may bind two different epitopes in the same domain. In one embodiment, the multi-specific antigen-binding molecule comprises a first antigen-binding specificity wherein the first binding specificity comprises the extracellular domain or fragment thereof of PD-1; and a second antigen-binding specificity to another epitope of PD-L1. In another embodiment, the multi-specific antigen-binding molecule comprises a first antigen-binding specificity wherein the first binding specificity comprises the extracellular domain or fragment thereof of B7-1; and a second antigen-binding specificity to another epitope of PD-L1.

In one embodiment, the invention provides a fully human monoclonal antibody or antigen-binding fragment thereof that binds to PD-L1, wherein the antibody or fragment thereof exhibits one or more of the following characteristics: (i) comprises a HCVR having an amino acid sequence selected from the group consisting of SEQ ID NO: 2, 18, 34, 50, 66, 82, 98, 114, 130, 146, 162, 178, 186, 202, 218, 234, 250, 258, 266, 274, 282, 290, 298, 306, 314, 322, 330 and 338, or a substantially similar sequence thereof having at least 90%, at least 95%, at least 98% or at least 99% sequence identity; (ii) comprises a LCVR having an amino acid sequence selected from the group consisting of SEQ ID NO: 10, 26, 42, 58, 74, 90, 106, 122, 138, 154, 170, 194, 210, 226, 242, 258, and 274, or a substantially similar sequence thereof having at least 90%, at least 95%, at least 98% or at least 99% sequence identity; (iii) comprises a HCDR3 domain having an amino acid sequence selected from the group consisting of SEQ ID NO: 8, 24, 40, 56, 72, 88, 104, 120, 136, 152, 168, 184, 192, 208, 224, 240, 256, 272, 280, 288, 296, 304, 312, 320, 328, 336 and 344, or a substantially similar sequence thereof having at least 90%, at least 95%, at least 98% or at least 99% sequence identity; and a LCDR3 domain having an amino acid sequence selected from the group consisting of SEQ ID NO: 16, 32, 48, 64, 80, 96, 112, 128, 144, 160, 176, 200, 216, 232, 248, 264, and 280, or a substantially similar sequence thereof having at least 90%, at least 95%, at least 98% or at least 99% sequence identity; (iv) comprises a HCDR1 domain having an amino acid sequence selected from the group consisting of SEQ ID NO: 4, 20, 36, 52, 68, 84, 100, 116, 132, 148, 164, 180, 188, 204, 220, 236, 252, 268, 284, 292, 300, 308, 316, 324, 332, and 340, or a substantially similar sequence thereof having at least 90%, at least 95%, at least 98% or at least 99% sequence identity; a HCDR2 domain having an amino acid sequence selected from the group consisting of SEQ ID NO: 6, 22, 38, 54, 70, 86, 102, 118, 134, 150, 166, 182, 190, 206, 222, 238, 254, 270, 286, 294, 302, 310, 318, 326, 334, and 342, or a substantially similar sequence thereof having at least 90%, at least 95%, at least 98% or at least 99% sequence identity; a LCDR1 domain having an amino acid sequence selected from the group consisting of SEQ ID NO: 12, 28, 44, 60, 76, 92, 108, 124, 140, 156, 172, 196, 212, 228, 244, 260, and 276, or a substantially similar sequence thereof having at least 90%, at least 95%, at least 98% or at least 99% sequence identity; and a LCDR2 domain having an amino acid sequence selected from the group consisting of SEQ ID NO: 14, 30, 46, 62, 78, 94, 110, 126, 142, 158, 174, 198, 214, 230, 246, 262, and 278, or a substantially similar sequence thereof having at least 90%, at least 95%, at least 98% or at least 99% sequence identity; (v) is a multi-specific antigen-binding molecule comprising a first binding specificity to PD-L1 and a second binding specificity to an antigen selected from the group consisting of PD-L1, a tumor specific antigen, a virally infected cell antigen, and a T-cell co-inhibitor; (vi) binds to human PD-L1 with a K_{D} of about 4 pM to about 645 nM; (vii) binds to cynomolgus PD-L1 with a K_{D} of about 70 pM to about 400 nM; (viii) blocks or enhances the binding of PD-L1 to PD-1 with an IC_{50 ≤ 770} pM; (ix) blocks or enhances the binding of PD-L1 to B7-1 with an IC_{50 ≤ 10} nM; (x) blocks PD-1-induced T-cell down-regulation and/or rescues T-cell signaling in a T-cell/APC luciferase reporter assay; (xi) stimulates T-cell proliferation and activity in a mixed lymphocyte reaction (MLR) assay; (xii) induces IL-2 and/or IFNγ production in a MLR assay; and (xiii) suppresses tumor growth and increases survival in subjects with cancer.

In one embodiment, the invention provides a fully human monoclonal antibody or antigen-binding fragment thereof that blocks PD-L1 binding to PD-1 or to B7-1, wherein the antibody or fragment thereof exhibits one or more of the following characteristics: (i) comprises a HCVR having an amino acid sequence selected from the group consisting of SEQ ID NO: 82, 98, 146, 162, 290, 306, 314, and 330, or a substantially similar sequence thereof having at least 90%, at least 95%, at least 98% or at least 99% sequence identity; (ii) comprises a LCVR having an amino acid sequence selected from the group consisting of SEQ ID NO: 90, 106, 154, 170, and 274, or a substantially similar sequence thereof having at least 90%, at least 95%, at least 98% or at least 99% sequence identity; (iii) comprises a HCDR3 domain having an amino acid sequence selected from the group consisting of SEQ ID NO: 88, 104, 152, 168, 296, 312, 320, and 336, or a substantially similar sequence thereof having at least 90%, at least 95%, at least 98% or at least 99% sequence identity; and a LCDR3 domain having an amino acid sequence selected from the group consisting of SEQ ID NO: 96, 112, 160, 176, and 280, or a substantially similar sequence thereof having at least 90%, at least 95%, at least 98% or at least 99% sequence identity; (iv) comprises a HCDR1 domain having an amino acid sequence selected from the group consisting of SEQ ID NO: 84, 100, 148, 164, 292, 308, 316, and 332, or a substantially similar sequence thereof having at least 90%, at least 95%, at least 98% or at least 99% sequence identity; a HCDR2 domain having an amino acid sequence selected from the group consisting of SEQ ID NO: 86, 102, 150, 166, 294, 310, 318, and 334, or a substantially similar sequence thereof having at least 90%, at least 95%, at least 98% or at least 99% sequence identity; a LCDR1 domain having an amino acid sequence selected from the group consisting of SEQ ID NO: 92, 108, 156, 172, and 276, or a substantially similar sequence thereof having at least 90%, at least 95%, at least 98% or at least 99% sequence identity; and a LCDR2 domain having an amino acid sequence selected from the group consisting of SEQ ID NO: 94, 110, 158, 174, and 278, or a substantially similar sequence thereof having at least 90%, at least 95%, at least 98% or at least 99% sequence identity; (v) is a multi-specific antigen-binding molecule comprising a first binding specificity to PD-L1 and a second binding specificity to an antigen selected from the group consisting of a different epitope of PD-L1, a tumor specific antigen, a virally-infected cell antigen, and a T-cell co-inhibitor; (vi) binds to human PD-L1 with a K_{D}≤10⁻¹⁰M; (vii) binds to cynomolgus PD-L1 with a K_{D}≤10⁻⁷M; (viii) blocks the binding of PD-L1 to PD-1 or to B7-1; (ix) blocks PD-1-induced T-cell down-regulation and/or rescues T-cell signaling in a T-cell/APC luciferase reporter assay; (xi) stimulates T-cell proliferation and activity in a mixed lymphocyte reaction (MLR) assay; (xii) induces IL-2 and/or IFNγ production in a MLR assay; and (xiii) suppresses tumor growth and increases survival in subjects with cancer.

The antibodies of the present invention may possess one or more of the aforementioned biological characteristics, or any combinations thereof. Other biological characteristics of the antibodies of the present invention will be evident to a person of ordinary skill in the art from a review of the present disclosure including the working Examples herein.

### Species Selectivity and Species Cross-Reactivity

According to certain embodiments of the invention, the anti-PD-L1 antibodies bind to human PD-L1 but not to PD-L1 from other species. Alternatively, the anti-PD-L1 antibodies of the invention, in certain embodiments, bind to human PD-L1 and to PD-L1 from one or more non-human species. For example, the anti-PD-L1 antibodies of the invention may bind to human PD-L1 and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee PD-L1. In certain embodiments, the anti-PD-L1 antibodies of the invention may bind to human and cynomolgus PD-L1 with the same affinities or with different affinities.

### Epitope Mapping and Related Technologies

The present invention includes anti-PD-L1 antibodies which interact with one or more amino acids found within one or more domains of the PD-L1 molecule including, e.g., extracellular (IgV-like) domain, the extracellular IgC-like domain, a transmembrane domain, and an intracellular domain. The epitope to which the antibodies bind may consist of a single contiguous sequence of 3 or more (e.g., 3, 4, 5, 6, 7, 8, 9, 10, 11, 12, 13, 14, 15, 16, 17, 18, 19, 20 or more) amino acids located within any of the aforementioned domains of the PD-L1 molecule (e.g. a linear epitope in a domain). Alternatively, the epitope may consist of a plurality of non-contiguous amino acids (or amino acid sequences) located within either or both of the aforementioned domains of the PD-L1 molecule (e.g. a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, NY). Other methods include alanine scanning mutational analysis, peptide blot analysis (Reineke (2004) Methods Mol. Biol. 248: 443-63), peptide cleavage analysis crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the invention into groups of antibodies binding different epitopes.

In certain embodiments, the anti-PD-L1 antibodies or antigen-binding fragments thereof bind an epitope within any one or more of the regions exemplified in PD-L1, either in natural form, as exemplified in SEQ ID NO: 351, or recombinantly produced, as exemplified in SEQ ID NOS: 345-348, or to a fragment thereof. In some embodiments, the antibodies of the invention bind to an extracellular region comprising one or more amino acids selected from the group consisting of amino acid residues 19-239 of PD-L1. In some embodiments, the antibodies of the invention bind to a region comprising one or more amino acids selected from the group consisting of amino acid residues 1-221 of cynomolgus PD-L1, as exemplified by SEQ ID NO: 346.

In certain embodiments, the antibodies of the invention, as shown in Table 1, interact with at least one amino acid sequence selected from the group consisting of amino acid residues ranging from about position 19 to about position 130 of SEQ ID NO: 351; or amino acid residues ranging from about position 130 to about position 153 of SEQ ID NO: 351; or amino acid residues ranging from about position 153 to about position 210 of SEQ ID NO: 351; or to amino acid residues ranging from about position 210 to about position 239 of SEQ ID NO: 351. These regions are partially exemplified in SEQ ID NOs: 345-348.

The present invention includes anti-PD-L1 antibodies that bind to the same epitope, or a portion of the epitope, as any of the specific exemplary antibodies described herein in Table 1, or an antibody having the CDR sequences of any of the exemplary antibodies described in Table 1. Likewise, the present invention also includes anti-PD-L1 antibodies that compete for binding to PD-L1 or a PD-L1 fragment with any of the specific exemplary antibodies described herein in Table 1, or an antibody having the CDR sequences of any of the exemplary antibodies described in Table 1. For example, the present invention includes anti-PD-L1 antibodies that cross-compete for binding to PD-L1 with one or more antibodies as defined in Example 6 herein (e.g., H2aM8309N, H1H9329P, H1H9336P, H2aM8314N, H2aM8316N, H2AM8718N, H1H9387P2, H1H9351P2, H1H9364P2, H1H9373P2, and H2aM8306N). The present invention also includes anti-PD-L1 antibodies that cross-compete for binding to PD-L1 with one or more antibodies as defined in Example 6 herein (e.g., H1H9396P2, H2aM8317N, H2aM8321N, H1H9323P, H1H9382P2, H1H9344P2, H1H9345P2 and H1H9354P2).

One can easily determine whether an antibody binds to the same epitope as, or competes for binding with, a reference anti-PD-L1 antibody by using routine methods known in the art. For example, to determine if a test antibody binds to the same epitope as a reference anti-PD-L1 antibody of the invention, the reference antibody is allowed to bind to a PD-L1 protein or peptide under saturating conditions. Next, the ability of a test antibody to bind to the PD-L1 molecule is assessed. If the test antibody is able to bind to PD-L1 following saturation binding with the reference anti-PD-L1 antibody, it can be concluded that the test antibody binds to a different epitope than the reference anti-PD-L1 antibody. On the other hand, if the test antibody is not able to bind to the PD-L1 protein following saturation binding with the reference anti-PD-L1 antibody, then the test antibody may bind to the same epitope as the epitope bound by the reference anti-PD-L1 antibody of the invention.

To determine if an antibody competes for binding with a reference anti-PD-L1 antibody, the above-described binding methodology is performed in two orientations: In a first orientation, the reference antibody is allowed to bind to a PD-L1 protein under saturating conditions followed by assessment of binding of the test antibody to the PD-L1 molecule. In a second orientation, the test antibody is allowed to bind to a PD-L1 molecule under saturating conditions followed by assessment of binding of the reference antibody to the PD-L1 molecule. If, in both orientations, only the first (saturating) antibody is capable of binding to the PD-L1 molecule, then it is concluded that the test antibody and the reference antibody compete for binding to PD-L1. As will be appreciated by a person of ordinary skill in the art, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50: 1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

### Immunoconjugates

The invention encompasses a human anti-PD-L1 monoclonal antibody conjugated to a therapeutic moiety (“immunoconjugate”), such as a cytotoxin or a chemotherapeutic agent to treat cancer. As used herein, the term “immunoconjugate” refers to an antibody which is chemically or biologically linked to a cytotoxin, a radioactive agent, a cytokine, an interferon, a target or reporter moiety, an enzyme, a toxin, a peptide or protein or a therapeutic agent. The antibody may be linked to the cytotoxin, radioactive agent, cytokine, interferon, target or reporter moiety, enzyme, toxin, peptide or therapeutic agent at any location along the molecule so long as it is able to bind its target. Examples of immunoconjugates include antibody drug conjugates and antibody-toxin fusion proteins. In one embodiment, the agent may be a second different antibody to PD-L1. In certain embodiments, the antibody may be conjugated to an agent specific for a tumor cell or a virally infected cell. The type of therapeutic moiety that may be conjugated to the anti-PD-L1 antibody and will take into account the condition to be treated and the desired therapeutic effect to be achieved. Examples of suitable agents for forming immunoconjugates are known in the art; see for example, WO 05/103081.

### Multi-Specific Antibodies

The antibodies of the present invention may be mono-specific, bi-specific, or multi-specific. Multi-specific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244.

In one aspect, the present invention includes multi-specific antigen-binding molecules or antigen-binding fragments thereof wherein one antigen-binding specificity of an immunoglobulin is specific for an epitope within the extracellular domain of PD-L1 (e.g., in the IgV-like region), or a fragment thereof, and the other antigen-binding specificity of the immunoglobulin is specific for binding to a different epitope in the extracellular domain of PD-L1 (e.g., in the IgC-like region), or a second therapeutic target, or is conjugated to a therapeutic moiety. In certain embodiments, the first antigen-binding specificity may comprise PD-1 or B7-1 or a fragment thereof. In one embodiment, the first antigen-binding specificity that binds to PD-L1 comprises the extracellular domain of PD-1. In certain embodiments of the invention, one antigen-binding specificity of an immunoglobulin is specific for an epitope within amino acid residues 19-239 of PD-L1 (SEQ ID NO: 351) or a fragment thereof, and the other specificity of the immunoglobulin is specific for a second target antigen. The second target antigen may be on the same cell as PD-L1 or on a different cell. In one embodiment, the second target cell is on an immune cell other than a T-cell such as a B-cell, antigen-presenting cell, monocyte, macrophage, or dendritic cell. In some embodiments, the second target antigen may be present on a tumor cell or on a virally infected cell.

In another aspect, the invention provides multi-specific antigen-binding molecules or antigen-binding fragments thereof comprising a first antigen-binding specificity that binds to PD-L1 and a second antigen-binding specificity that binds specifically to a target antigen on a tumor cell. In various embodiments, the tumor-specific antigen is one of CA9, CA125, melanoma-associated antigen (MAGE), carcinoembryonic antigen (CEA), vimentin, tumor-M2-PK, prostate-specific antigen (PSA), MART-1, or CA19-9. Non-limiting examples of other specific tumor-associated antigens include, e.g., AFP, ALK, BAGE proteins, B-catenin, brc-abl, BRCA1, BORIS, carbonic anhydrase IX, caspase-8, CCR5, CD19, CD20, CD30, CD40, CDK4, CTLA4, cyclin-B1, CYP1B1, EGFR, EGFRvIII, ErbB2/Her2, ErbB3, ErbB4, ETV6-AML, EpCAM, EphA2, Fra-1, FOLR1, GAGE proteins (e.g., GAGE-1,-2), GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and-12), HLA-A2, MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, Steap-1, Steap-2, survivin, TAG-72, TGF-β, TMPRSS2, Tn, TRP-1, TRP-2, tyrosinase, and uroplakin-3. In other embodiments, the second antigen-binding specificity binds to a tumor antigen that is present on tumor cells specific to, but not limited to, renal cell carcinoma, prostate cancer, colorectal cancer, melanoma, breast cancer, kidney cancer, ovarian cancer, and pancreatic cancer. The antibodies of the invention, in this aspect, may inhibit the activity of PD-L1.

In another aspect, the invention provides multi-specific antigen-binding molecules or antigen-binding fragments thereof wherein the second antigen-binding specificity binds to an antigen specific to a virally-infected cell. In certain embodiments, the second antigen-binding specificity binds to an antigen specific to a cell infected with a virus selected from the group consisting of HIV, HBV, HCV, HPV, LCMV and SIV.

In another aspect, the invention provides multi-specific antigen-binding molecules or antigen-binding fragments thereof comprising a first antigen-binding specificity that binds to PD-L1 and a second antigen-binding specificity that binds to a T-cell co-inhibitor such as PD-1, LAG-3, TIM3, B7-1, CTLA-4, BTLA, CD-28, 2B4, LY108, TIGIT, LAIR1, ICOS and CD160.

Any of the multi-specific antigen-binding molecules, or variants thereof, may be constructed using standard molecular biological techniques (e.g., recombinant DNA and protein expression technology), as will be well known to a person of ordinary skill in the art.

In some embodiments, PD-L1-specific antibodies are generated in a bi-specific format (a “bi-specific”) in which variable regions binding to distinct domains of PD-L1 are linked together to confer dual-domain specificity within a single binding molecule. Appropriately designed bi-specifics may enhance overall PD-L1 inhibitory efficacy through increasing both specificity and binding avidity. Variable regions with specificity for individual domains, (e.g., segments of the N-terminal domain), or that can bind to different regions within one domain, are paired on a structural scaffold that allows each region to bind simultaneously to the separate epitopes, or to different regions within one domain. In one example for a bi-specific, heavy chain variable regions (V_{H}) from a binder with specificity for one domain are recombined with light chain variable regions (V_{L}) from a series of binders with specificity for a second domain to identify non-cognate V_{L} partners that can be paired with an original V_{H} without disrupting the original specificity for that V_{H}. In this way, a single V_{L} segment (e.g., V_{L}1) can be combined with two different V_{H} domains (e.g., V_{H}1 and V_{H}2) to generate a bi-specific comprised of two binding “arms” (V_{H}1-V_{L}1 and V_{H}2-V_{L}1). Use of a single V_{L} segment reduces the complexity of the system and thereby simplifies and increases efficiency in cloning, expression, and purification processes used to generate the bi-specific (See, for example, U.S. Ser. No. 13/022,759 and US2010/0331527).

Alternatively, antibodies that bind more than one domains and a second target, such as, but not limited to, for example, a second different anti-PD-L1 antibody, may be prepared in a bi-specific format using techniques described herein, or other techniques known to those skilled in the art. Antibody variable regions binding to distinct regions may be linked together with variable regions that bind to relevant sites on, for example, the extracellular domain of PD-L1, to confer dual-antigen specificity within a single binding molecule. Appropriately designed bi-specifics of this nature serve a dual function. Variable regions with specificity for the extracellular domain are combined with a variable region with specificity for outside the extracellular domain and are paired on a structural scaffold that allows each variable region to bind to the separate antigens.

An exemplary bi-specific antibody format that can be used in the context of the present invention involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bi-specific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies. Variations on the bi-specific antibody format described above are contemplated within the scope of the present invention.

Other exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab2 bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

### Therapeutic Administration and Formulations

The invention provides therapeutic compositions comprising the anti-PD-L1 antibodies or antigen-binding fragments thereof of the present invention. Therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody may vary depending upon the age and the size of a subject to be administered, target disease, conditions, route of administration, and the like. When an antibody of the present invention is used for treating a disease or disorder in an adult patient, or for preventing such a disease, it is advantageous to administer the antibody of the present invention normally at a single dose of about 0.1 to about 100 mg/kg body weight, more preferably about 5 to about 80, about 10 to about 60, or about 20 to about 50 mg/kg body weight. Depending on the severity of the condition, the frequency and the duration of the treatment can be adjusted. In certain embodiments, the antibody or antigen-binding fragment thereof of the invention can be administered as an initial dose of at least about 0.1 mg to about 800 mg, about 1 to about 500 mg, about 5 to about 300 mg, or about 10 to about 200 mg, to about 100 mg, or to about 50 mg. In certain embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can be also delivered in a vesicle, in particular a liposome (see, for example, Langer (1990) Science 249:1527-1533).

The use of nanoparticles to deliver the antibodies of the present invention is also contemplated herein. Antibody-conjugated nanoparticles may be used both for therapeutic and diagnostic applications. Antibody-conjugated nanoparticles and methods of preparation and use are described in detail by Arruebo, M., et al. 2009 (“Antibody-conjugated nanoparticles for biomedical applications” in J. Nanomat. Volume 2009, Article ID 439389, 24 pages, doi: 10.1155/2009/439389), incorporated herein by reference. Nanoparticles may be developed and conjugated to antibodies contained in pharmaceutical compositions to target tumor cells or autoimmune tissue cells or virally infected cells. Nanoparticles for drug delivery have also been described in, for example, U.S. Pat. Nos. 8,257,740, or 8,246,995, each incorporated herein in its entirety.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous, intracranial, intraperitoneal and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but certainly are not limited to the SOLOSTART pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.) and the HUMIRA™ Pen (Abbott Labs, Abbott Park, IL), to name only a few.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 500 mg per dosage form in a unit dose; especially in the form of injection, it is preferred that the antibody is contained in about 5 to about 100 mg and in about 10 to about 250 mg for the other dosage forms.

### Therapeutic Uses of the Antibodies

The antibodies of the present invention are useful for the treatment, prevention, and/or amelioration of disease or disorder or condition such as cancer, autoimmune disease or a viral infection and/or for ameliorating at least one symptom associated with such disease, disorder or condition. In some embodiments of the invention, the antibodies described herein are useful for treating subjects suffering from primary or recurrent cancer, including for example, renal cell carcinoma, prostate cancer, ovarian cancer, kidney cancer, colorectal cancer, gastric cancer, breast cancer, head and neck cancer, non-small-cell lung cancer, brain cancer, multiple myeloma, and melanoma. The antibodies may be used to treat early stage or late-stage symptoms of cancer. In one embodiment, an antibody or fragment thereof of the invention may be used to treat metastatic cancer. The antibodies are useful in reducing or inhibiting or shrinking tumor growth of both solid tumors and blood cancers. In certain embodiments, the antibodies may be used to prevent relapse of a tumor. In certain embodiments, treatment with an antibody or antigen-binding fragment thereof of the invention may lead to more than 50% regression, more than 60% regression, more than 70% regression, more than 80% regression or more than 90% regression of a tumor in a subject. In certain embodiments, the antibodies may be used to increase survival of a subject suffering from cancer.

In certain embodiments, the antibodies of the invention are useful to treat subjects suffering from a chronic viral infection. In some embodiments, the antibodies of the invention are useful in decreasing viral titers in the host and/or rescuing exhausted T-cells. In one embodiment, an antibody or antigen-binding fragment thereof the invention may be administered at a therapeutic dose to a patient with an infection by human immunodeficiency virus (HIV) or human papilloma virus (HPV) or hepatitis B/C virus (HBV/HCV). In a related embodiment, an antibody or antigen-binding fragment thereof of the invention may be used to treat an infection by simian immunodeficiency virus (SIV) in a simian subject such as cynomolgus. In another embodiment, an antibody or fragment thereof of the invention may be used to treat chronic viral infection by lymphocytic choriomeningitis virus (LCMV).

In certain embodiments, a blocking antibody of the present invention may be administered in a therapeutically effective amount to a subject suffering from cancer or a viral infection.

In certain embodiments, the antibodies of the invention are useful for treating an autoimmune disease, including but not limited to, alopecia areata, autoimmune hepatitis, celiac disease, Graves' disease, Guillain-Barre syndrome, Hashimoto's disease, hemolytic anemia, inflammatory bowel disease, inflammatory myopathies, multiple sclerosis, primary biliary cirrhosis, psoriasis, rheumatoid arthritis, scleroderma, Sjögren's syndrome, systemic lupus erthyematosus, vitiligo, autoimmune pancreatitis, autoimmune urticaria, autoimmune thrombocytopenia purpura, Crohn's disease, diabetes type I, eosinophilic fasciitis, eosinophilic enterogastritis, Goodpasture's syndrome, myasthenia gravis, psoriatic arthritis, rheumatic fever, ulcerative colitis, vasculitis and Wegener's granulomatosis. In certain embodiments, an activating antibody of the invention may be used to treat a subject suffering from autoimmune disease.

One or more antibodies of the present invention may be administered to relieve or prevent or decrease the severity of one or more of the symptoms or conditions of the disease or disorder.

It is also contemplated herein to use one or more antibodies of the present invention prophylactically to patients at risk for developing a disease or disorder such as cancer, and chronic viral infection.

In a further embodiment of the invention the present antibodies are used for the preparation of a pharmaceutical composition for treating patients suffering from cancer, autoimmune disease or viral infection. In another embodiment of the invention, the present antibodies are used as adjunct therapy with any other agent or any other therapy known to those skilled in the art useful for treating cancer, autoimmune disease or viral infection.

### Combination Therapies

Combination therapies may include an anti-PD-L1 antibody of the invention and any additional therapeutic agent that may be advantageously combined with an antibody of the invention, or with a biologically active fragment of an antibody of the invention.

The antibodies of the present invention may be combined synergistically with one or more anti-cancer drugs or therapy used to treat cancer, including, for example, renal cell carcinoma, ovarian cancer, prostate cancer, colorectal cancer, non-small-cell lung cancer, and melanoma. It is contemplated herein to use anti-PD-L1 antibodies of the invention in combination with immunostimulatory and/or immunosupportive therapies to inhibit tumor growth, and/or enhance survival of cancer patients. The immunostimulatory therapies include direct immunostimulatory therapies to augment immune cell activity by either “releasing the brake” on suppressed immune cells or “stepping on the gas” to activate an immune response. Examples include targeting other checkpoint receptors, vaccination and adjuvants. The immunosupportive modalities may increase antigenicity of the tumor by promoting immunogenic cell death, inflammation or have other indirect effects that promote an anti-tumor immune response. Examples include radiation, chemotherapy, anti-angiogenic agents, and surgery.

In various embodiments, one or more antibodies of the present invention may be used in combination with a second antibody to PD-L1, an antibody to PD-1 (e.g., nivolumab), a LAG-3 inhibitor, a CTLA-4 inhibitor (e.g., ipilimumab), a TIM3 inhibitor, a BTLA inhibitor, a TIGIT inhibitor, a CD47 inhibitor, an antagonist of another T-cell co-inhibitor or ligand (e.g., an antibody to CD-28, 2B4, LY108, LAIR1, ICOS, CD160 or VISTA), an indoleamine-2,3-dioxygenase (IDO) inhibitor, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a transforming growth factor beta (TGFβ) inhibitor, an epidermal growth factor receptor (EGFR) inhibitor (e.g., erlotinib, cetuximab), an agonist to a co-stimulatory receptor (e.g., an agonist to glucocorticoid-induced TNFR-related protein), an antibody to a tumor-specific antigen (e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9), a vaccine (e.g., Bacillus Calmette-Guerin, a cancer vaccine), an adjuvant to increase antigen presentation (e.g., granulocyte-macrophage colony-stimulating factor), a bispecific antibody (e.g., CD3xCD20 bispecific antibody, PSMAxCD3 bispecific antibody), a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), cyclophosphamide, radiotherapy, an IL-6R inhibitor (e.g., sarilumab), an IL-4R inhibitor (e.g., dupilumab), an IL-10 inhibitor, a cytokine such as IL-2, IL-7, IL-21, and IL-15, an antibody-drug conjugate (ADC) (e.g., anti-CD19-DM4 ADC, and anti-DS6-DM4 ADC), an anti-inflammatory drug (e.g., corticosteroids, and non-steroidal anti-inflammatory drugs), a dietary supplement such as anti-oxidants or any palliative care to treat cancer. In certain embodiments, the anti-PD-L1 antibodies of the present invention may be used in combination with cancer vaccines including dendritic cell vaccines, oncolytic viruses, tumor cell vaccines, etc. to augment the anti-tumor response. Examples of cancer vaccines that can be used in combination with anti-PD-L1 antibodies of the present invention include MAGE3 vaccine for melanoma and bladder cancer, MUC1 vaccine for breast cancer, EGFRv3 (e.g., Rindopepimut) for brain cancer (including glioblastoma multiforme), or ALVAC-CEA (for CEA+ cancers). In certain embodiments, the anti-PD-L1 antibodies of the present invention may be used in combination with a dietary supplement such as anti-oxidants or any palliative care to treat cancer.

In certain embodiments, the anti-PD-L1 antibodies of the invention may be administered in combination with radiation therapy in methods to generate long-term durable anti-tumor responses and/or enhance survival of patients with cancer. In some embodiments, the anti-PD-L1 antibodies of the invention may be administered prior to, concomitantly or after administering radiation therapy to a cancer patient. For example, radiation therapy may be administered in one or more doses to tumor lesions followed by administration of one or more doses of anti-PD-L1 antibodies of the invention. In some embodiments, radiation therapy may be administered locally to a tumor lesion to enhance the local immunogenicity of a patient's tumor (adjuvinating radiation) and/or to kill tumor cells (ablative radiation) followed by systemic administration of an anti-PD-L1 antibody of the invention. For example, intracranial radiation may be administered to a patient with brain cancer (e.g., glioblastoma multiforme) along with systemic administration of an anti-PD-L1 antibody of the invention. In certain embodiments, the anti-PD-L1 antibodies of the invention may be administered in combination with radiation therapy and a chemotherapeutic agent (e.g., temozolomide) or a VEGF antagonist (e.g., aflibercept).

The antibodies or fragments thereof of the invention may be administered in combination with one or more anti-viral drugs known in the art, including but not limited to, zidovudine, lamivudine, abacavir, ribavirin, lopinavir, efavirenz, cobicistat, tenofovir, rilpivirine and corticosteroids. In some embodiments, the anti-PD-L1 antibodies of the invention may be administered in combination with a LAG3 inhibitor, a CTLA-4 inhibitor, a PD-1 inhibitor or any antagonist of another T-cell co-inhibitor to treat chronic viral infection.

The antibodies of fragments thereof of the invention may be used in combination with any drug or therapy known in the art (e.g., corticosteroids and other immunosuppressants) to treat an autoimmune disease or disorder including, but not limited to, alopecia areata, autoimmune hepatitis, celiac disease, Graves' disease, Guillain-Barre syndrome, Hashimoto's disease, hemolytic anemia, inflammatory bowel disease, inflammatory myopathies, multiple sclerosis, primary biliary cirrhosis, psoriasis, rheumatoid arthritis, scleroderma, Sjögren's syndrome, systemic lupus erthyematosus, vitiligo, autoimmune pancreatitis, autoimmune urticaira, autoimmune thrombocytopenia purpura, Crohn's disease, diabetes type I, eosinophilic fasciitis, eosinophilic enterogastritis, Goodpasture's syndrome, myasthenia gravis, psoriatic arthritis, rheumatic fever, ulcerative colitis, vasculitis and Wegener's granulomatosis.

The additional therapeutically active component(s) may be administered prior to, concurrent with, or after the administration of the anti-PD-L1 antibody of the present invention. For purposes of the present disclosure, such administration regimens are considered the administration of an anti-PD-L1 antibody “in combination with” a second therapeutically active component.

The additional therapeutically active component(s) may be administered to a subject prior to administration of an anti-PD-L1 antibody of the present invention. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, 15 minutes before, 10 minutes before, 5 minutes before, or less than 1 minute before administration of the second component. In other embodiments, the additional therapeutically active component(s) may be administered to a subject after administration of an anti-PD-L1 antibody of the present invention. For example, a first component may be deemed to be administered “after” a second component if the first component is administered 1 minute after, 5 minutes after, 10 minutes after, 15 minutes after, 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after administration of the second component. In yet other embodiments, the additional therapeutically active component(s) may be administered to a subject concurrent with administration of an anti-PD-L1 antibody of the present invention. “Concurrent” administration, for purposes of the present invention, includes, e.g., administration of an anti-PD-L1 antibody and an additional therapeutically active component to a subject in a single dosage form (e.g., co-formulated), or in separate dosage forms administered to the subject within about 30 minutes or less of each other. If administered in separate dosage forms, each dosage form may be administered via the same route (e.g., both the anti-PD-L1 antibody and the additional therapeutically active component may be administered intravenously, subcutaneously, etc.); alternatively, each dosage form may be administered via a different route (e.g., the anti-PD-L1 antibody may be administered intravenously, and the additional therapeutically active component may be administered subcutaneously). In any event, administering the components in a single dosage from, in separate dosage forms by the same route, or in separate dosage forms by different routes are all considered “concurrent administration,” for purposes of the present disclosure. For purposes of the present disclosure, administration of an anti-PD-L1 antibody “prior to”, “concurrent with,” or “after” (as those terms are defined herein above) administration of an additional therapeutically active component is considered administration of an anti-PD-L1 antibody “in combination with” an additional therapeutically active component).

The present invention includes pharmaceutical compositions in which an anti-PD-L1 antibody of the present invention is co-formulated with one or more of the additional therapeutically active component(s) as described elsewhere herein using a variety of dosage combinations.

In exemplary embodiments in which an anti-PD-L1 antibody of the invention is administered in combination with a VEGF antagonist (e.g., a VEGF trap such as aflibercept), including administration of co-formulations comprising an anti-PD-L1 antibody and a VEGF antagonist, the individual components may be administered to a subject and/or co-formulated using a variety of dosage combinations. For example, the anti-PD-L1 antibody may be administered to a subject and/or contained in a co-formulation in an amount selected from the group consisting of 0.01 mg, 0.02 mg, 0.03 mg, 0.04 mg, 0.05 mg, 0.1 mg, 0.2 mg, 0.3 mg, 0.4 mg, 0.5 mg, 0.6 mg, 0.7 mg, 0.8 mg, 0.9 mg, 1.0 mg, 1.5 mg, 2.0 mg, 2.5 mg, 3.0 mg, 3.5 mg, 4.0 mg, 4.5 mg, 5.0 mg, 6.0 mg, 7.0 mg, 8.0 mg, 9.0 mg, and 10.0 mg; and the VEGF antagonist (e.g., a VEGF trap such as aflibercept) may be administered to the subject and/or contained in a co-formulation in an amount selected from the group consisting of 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg and 3.0 mg. The combinations/co-formulations may be administered to a subject according to any of the administration regimens disclosed elsewhere herein, including, e.g., twice a week, once every week, once every 2 weeks, once every 3 weeks, once every month, once every 2 months, once every 3 months, once every 4 months, once every 5 months, once every 6 months, etc.

### Administrative Regimens

According to certain embodiments of the present invention, multiple doses of an anti-PD-L1 antibody (or a pharmaceutical composition comprising a combination of an anti-PD-L1 antibody and any of the additional therapeutically active agents mentioned herein) may be administered to a subject over a defined time course. The methods according to this aspect of the invention comprise sequentially administering to a subject multiple doses of an anti-PD-L1 antibody of the invention. As used herein, “sequentially administering” means that each dose of anti-PD-L1 antibody is administered to the subject at a different point in time, e.g., on different days separated by a predetermined interval (e.g., hours, days, weeks or months). The present invention includes methods which comprise sequentially administering to the patient a single initial dose of an anti-PD-L1 antibody, followed by one or more secondary doses of the anti-PD-L1 antibody, and optionally followed by one or more tertiary doses of the anti-PD-L1 antibody. The anti-PD-L1 antibody may be administered at a dose of between 0.1 mg/kg to about 100 mg/kg.

The terms “initial dose,” “secondary doses,” and “tertiary doses,” refer to the temporal sequence of administration of the anti-PD-L1 antibody of the invention. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of anti-PD-L1 antibody, but generally may differ from one another in terms of frequency of administration. In certain embodiments, however, the amount of anti-PD-L1 antibody contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present invention, each secondary and/or tertiary dose is administered 1 to 26 (e.g., 1, 1 ½, 2, 2 ½, 3, 3 ½, 4, 4 ½, 5, 5 ½, 6, 6 ½, 7, 7 ½, 8, 8 ½, 9, 9 ½, 10, 10 ½, 11, 11 ½, 12, 12 ½, 13, 13 ½, 14, 14 ½, 15, 15 ½, 16, 16 ½, 17, 17 ½, 18, 18 ½, 19, 19 ½, 20, 20 ½, 21, 21 ½, 22, 22 ½, 23, 23 ½, 24, 24 ½, 25, 25 ½, 26, 26 ½, or more) weeks after the immediately preceding dose. The phrase “the immediately preceding dose,” as used herein, means, in a sequence of multiple administrations, the dose of anti-PD-L1 antibody which is administered to a patient prior to the administration of the very next dose in the sequence with no intervening doses.

The methods according to this aspect of the invention may comprise administering to a patient any number of secondary and/or tertiary doses of an anti-PD-L1 antibody. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in embodiments involving multiple tertiary doses, each tertiary dose may be administered at the same frequency as the other tertiary doses. For example, each tertiary dose may be administered to the patient 2 to 12 weeks after the immediately preceding dose. In certain embodiments of the invention, the frequency at which the secondary and/or tertiary doses are administered to a patient can vary over the course of the treatment regimen. The frequency of administration may also be adjusted during the course of treatment by a physician depending on the needs of the individual patient following clinical examination.

The present invention includes administration regimens in which 2 to 6 loading doses are administered to a patient at a first frequency (e.g., once a week, once every two weeks, once every three weeks, once a month, once every two months, etc.), followed by administration of two or more maintenance doses to the patient on a less frequent basis. For example, according to this aspect of the invention, if the loading doses are administered at a frequency of, e.g., once a month (e.g., two, three, four, or more loading doses administered once a month), then the maintenance doses may be administered to the patient once every five weeks, once every six weeks, once every seven weeks, once every eight weeks, once every ten weeks, once every twelve weeks, etc.).

Diagnostic Uses of the Antibodies

The anti-PD-L1 antibodies of the present invention may be used to detect and/or measure PD-L1 in a sample, e.g., for diagnostic purposes. Some embodiments contemplate the use of one or more antibodies of the present invention in assays to detect a disease or disorder such as cancer, autoimmune disease or chronic viral infection. Exemplary diagnostic assays for PD-L1 may comprise, e.g., contacting a sample, obtained from a patient, with an anti-PD-L1 antibody of the invention, wherein the anti-PD-L1 antibody is labeled with a detectable label or reporter molecule or used as a capture ligand to selectively isolate PD-L1 from patient samples. Alternatively, an unlabeled anti-PD-L1 antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure PD-L1 in a sample include enzyme-linked immunosorbent assay (ELISA), radioimmunoassay (RIA), and fluorescence-activated cell sorting (FACS).

Samples that can be used in PD-L1 diagnostic assays according to the present invention include any tissue or fluid sample obtainable from a patient, which contains detectable quantities of either PD-L1 protein, or fragments thereof, under normal or pathological conditions. Generally, levels of PD-L1 in a particular sample obtained from a healthy patient (e.g., a patient not afflicted with cancer or an autoimmune disease) will be measured to initially establish a baseline, or standard, level of PD-L1. This baseline level of PD-L1 can then be compared against the levels of PD-L1 measured in samples obtained from individuals suspected of having a cancer-related condition, or symptoms associated with such condition.

The antibodies specific for PD-L1 may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface.

Aspects of the invention relate to use of the disclosed antibodies as markers for predicting prognosis of cancer or an autoimmune disorder in patients. Antibodies of the present invention may be used in diagnostic assays to evaluate prognosis of cancer in a patient and to predict survival.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Generation of Human Antibodies to PD-L1

Human antibodies to PD-L1 were generated using a fragment of PD-L1 that ranges from about amino acids 19-239 of SEQ ID NO: 351 (Genbank Accession No. NP_054862.1). The immunogen was administered directly, with an adjuvant to stimulate the immune response, to a VELOCIMMUNE® mouse comprising DNA encoding human Immunoglobulin heavy and kappa light chain variable regions. The antibody immune response was monitored by a PD-L1-specific immunoassay. When a desired immune response was achieved splenocytes were harvested and fused with mouse myeloma cells to preserve their viability and form hybridoma cell lines. The hybridoma cell lines were screened and selected to identify cell lines that produce PD-L1-specific antibodies. Using this technique, and the immunogen described above, several anti-PD-L1 chimeric antibodies (i.e., antibodies possessing human variable domains and mouse constant domains) were obtained; exemplary antibodies generated in this manner were designated as H2M8306N, H2M8307N, H2M8309N, H2M8310N, H2M8312N, H2M8314N, H2M8316N, H2M8317N, H2M8321N, H2M8323N, H2M8718N, H2M8718N2, and H2M8719N.

Anti-PD-L1 antibodies were also isolated directly from antigen-positive B cells without fusion to myeloma cells, as described in U.S. 2007/0280945A1, herein specifically incorporated by reference in its entirety. Using this method, several fully human anti-PD-L1 antibodies (i.e., antibodies possessing human variable domains and human constant domains) were obtained; exemplary antibodies generated in this manner were designated as follows: H1H9323P, H1H9327P, H1H9329P, H1H9336P, H1H9344P2, H1H9345P2, H1H9351P2, H1H9354P2, H1H9364P2, H1H9373P2, H1H9382P2, H1H9387P2, and H1H9396P2.

The biological properties of the exemplary antibodies generated in accordance with the methods of this Example are described in detail in the Examples set forth below.

### Example 2: Heavy and Light Chain Variable Region Amino Acid and Nucleotide Sequences

Table 1 sets forth the amino acid sequence identifiers of the heavy and light chain variable regions and CDRs of selected anti-PD-L1 antibodies of the invention. The corresponding nucleic acid sequence identifiers are set forth in Table 2.

TABLE 1
Amino Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H2M8306N | 2 | 4 | 6 | 8 | 10 | 12 | 14 | 16 |
| H2M8307N | 18 | 20 | 22 | 24 | 26 | 28 | 30 | 32 |
| H2M8309N | 34 | 36 | 38 | 40 | 42 | 44 | 46 | 48 |
| H2M8310N | 50 | 52 | 54 | 56 | 58 | 60 | 62 | 64 |
| H2M8312N | 66 | 68 | 70 | 72 | 74 | 76 | 78 | 80 |
| H2M8314N | 82 | 84 | 86 | 88 | 90 | 92 | 94 | 96 |
| H2M8316N | 98 | 100 | 102 | 104 | 106 | 108 | 110 | 112 |
| H2M8317N | 114 | 116 | 118 | 120 | 122 | 124 | 126 | 128 |
| H2M8321N | 130 | 132 | 134 | 136 | 138 | 140 | 142 | 144 |
| H2M8323N | 146 | 148 | 150 | 152 | 154 | 156 | 158 | 160 |
| H2M8718N | 162 | 164 | 166 | 168 | 170 | 172 | 174 | 176 |
| H2M8718N2 | 178 | 180 | 182 | 184 | 170 | 172 | 174 | 176 |
| H2M8719N | 186 | 188 | 190 | 192 | 194 | 196 | 198 | 200 |
| H1H9323P | 202 | 204 | 206 | 208 | 210 | 212 | 214 | 216 |
| H1H9327P | 218 | 220 | 222 | 224 | 226 | 228 | 230 | 232 |
| H1H9329P | 234 | 236 | 238 | 240 | 242 | 244 | 246 | 248 |
| H1H9336P | 250 | 252 | 254 | 256 | 258 | 260 | 262 | 264 |
| H1H9344P2 | 266 | 268 | 270 | 272 | 274 | 276 | 278 | 280 |
| H1H9345P2 | 282 | 284 | 286 | 288 | 274 | 276 | 278 | 280 |
| H1H9351P2 | 290 | 292 | 294 | 296 | 274 | 276 | 278 | 280 |
| H1H9354P2 | 298 | 300 | 302 | 304 | 274 | 276 | 278 | 280 |
| H1H9364P2 | 306 | 308 | 310 | 312 | 274 | 276 | 278 | 280 |
| H1H9373P2 | 314 | 316 | 318 | 320 | 274 | 276 | 278 | 280 |
| H1H9382P2 | 322 | 324 | 326 | 328 | 274 | 276 | 278 | 280 |
| H1H9387P2 | 330 | 332 | 334 | 336 | 274 | 276 | 278 | 280 |
| H1H9396P2 | 338 | 340 | 342 | 344 | 274 | 276 | 278 | 280 |

TABLE 2
Nucleic Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H2M8306N | 1 | 3 | 5 | 7 | 9 | 11 | 13 | 15 |
| H2M8307N | 17 | 19 | 21 | 23 | 25 | 27 | 29 | 31 |
| H2M8309N | 33 | 35 | 37 | 39 | 41 | 43 | 45 | 47 |
| H2M8310N | 49 | 51 | 53 | 55 | 57 | 59 | 61 | 63 |
| H2M8312N | 65 | 67 | 69 | 71 | 73 | 75 | 77 | 79 |
| H2M8314N | 81 | 83 | 85 | 87 | 89 | 91 | 93 | 95 |
| H2M8316N | 97 | 99 | 101 | 103 | 105 | 107 | 109 | 111 |
| H2M8317N | 113 | 115 | 117 | 119 | 121 | 123 | 125 | 127 |
| H2M8321N | 129 | 131 | 133 | 135 | 137 | 139 | 141 | 143 |
| H2M8323N | 145 | 147 | 149 | 151 | 153 | 155 | 157 | 159 |
| H2M8718N | 161 | 163 | 165 | 167 | 169 | 171 | 173 | 175 |
| H2M8718N2 | 177 | 179 | 181 | 183 | 169 | 171 | 173 | 175 |
| H2M8719N | 185 | 187 | 189 | 191 | 193 | 195 | 197 | 199 |
| H1H9323P | 201 | 203 | 205 | 207 | 209 | 211 | 213 | 215 |
| H1H9327P | 217 | 219 | 221 | 223 | 225 | 227 | 229 | 231 |
| H1H9329P | 233 | 235 | 237 | 239 | 241 | 243 | 245 | 247 |
| H1H9336P | 249 | 251 | 253 | 255 | 257 | 259 | 261 | 263 |
| H1H9344P2 | 265 | 267 | 269 | 271 | 273 | 275 | 277 | 279 |
| H1H9345P2 | 281 | 283 | 285 | 287 | 273 | 275 | 277 | 279 |
| H1H9351P2 | 289 | 291 | 293 | 295 | 273 | 275 | 277 | 279 |
| H1H9354P2 | 297 | 299 | 301 | 303 | 273 | 275 | 277 | 279 |
| H1H9364P2 | 305 | 307 | 309 | 311 | 273 | 275 | 277 | 279 |
| H1H9373P2 | 313 | 315 | 317 | 319 | 273 | 275 | 277 | 279 |
| H1H9382P2 | 321 | 323 | 325 | 327 | 273 | 275 | 277 | 279 |
| H1H9387P2 | 329 | 331 | 333 | 335 | 273 | 275 | 277 | 279 |
| H1H9396P2 | 337 | 339 | 341 | 343 | 273 | 275 | 277 | 279 |

Antibodies are typically referred to herein according to the following nomenclature: Fc prefix (e.g. “H1H,” “H2M,” “H2aM,” etc.), followed by a numerical identifier (e.g. “8306,” “9323,” etc., as shown in Table 1), followed by a “P,” “N,” “P2,” or “N2” suffix. Thus, according to this nomenclature, an antibody may be referred to herein as, e.g., “H2M8306N,” “H1H9344P2,” etc. The H1H, H2M and H2aM prefixes on the antibody designations used herein indicate the particular Fc region isotype of the antibody. For example, an “H1H” antibody has a human IgG1 Fc, an “H1M” antibody has a mouse IgG1 Fc, and an “H2M” or “H2aM” antibody has a mouse IgG2 Fc, (all variable regions are fully human as denoted by the first ‘H’ in the antibody designation). As will be appreciated by a person of ordinary skill in the art, an antibody having a particular Fc isotype can be converted to an antibody with a different Fc isotype (e.g., an antibody with a mouse IgG1 Fc can be converted to an antibody with a human IgG4, etc.), but in any event, the variable domains (including the CDRs)—which are indicated by the numerical identifiers shown in Table 1—will remain the same, and the binding properties to antigen are expected to be identical or substantially similar regardless of the nature of the Fc domain.

### Example 3: Antibody Binding to PD-L1 as Determined by Surface Plasmon Resonance

Binding association and dissociation rate constants (kₐ and k_{d}, respectively), equilibrium dissociation constants and dissociation half-lives (K_{D} and t_{1/2}, respectively) of antigen binding to purified anti-PD-L1 monoclonal antibodies were determined using a real-time surface plasmon resonance biosensor assay on a Biacore 4000 instrument. The Biacore sensor surface was either derivatized with polyclonal rabbit anti-mouse antibody (GE, #BR-1008-38) or with monoclonal mouse anti-human Fc antibody (GE, #BR-1008-39) to capture approximately 200-300 RUs of anti-PD-L1 monoclonal antibodies, expressed with either a mouse Fc or with human Fc, respectively. The PD-L1 reagents tested for binding to the anti-PD-L1 antibodies included recombinant human PD-L1 (amino acids 19-239 of accession number NP_054862.1) expressed with a C-terminal myc-myc-hexahistidine tag (hPD-L1-MMH; SEQ ID: 345), recombinant cynomolgus monkey PD-L1 expressed with a C-terminal myc-myc-hexahistidine tag (MfPD-L1-MMH; SEQ ID: 346), recombinant human PD-L1 (amino acids 19-239 of accession number NP_054862.1) expressed with either a C-terminal human IgG1 Fc tag (hPD-L1-hFc; SEQ ID: 347) or with a C-terminal mouse IgG2a Fc tag (hPD-L1-mFc; SEQ ID: 348), and recombinant cynomolgus monkey PD-L1 expressed with a C-terminal mouse IgG2a Fc tag (MfPD-L1-mFc; SEQ ID: 353). Different concentrations of PD-L1 reagents were injected over the anti-PD-L1 monoclonal antibody captured surface at a flow rate of 30 μL/min. The binding of the PD-L1 reagents to the captured monoclonal antibodies was monitored for 3 to 4 minutes while the dissociation of antibody bound PD-L1 reagents was monitored for 10 minutes in HBST running buffer (0.01 M HEPES pH 7.4, 0.15 M NaCl, 3 mM EDTA, 0.05% v/v Surfactant tween-20). Experiments were performed at either 25° C. or 37° C. Kinetic association (kₐ) and dissociation (k_{d}) rate constants were determined by processing and fitting the data to a 1:1 binding model using Scrubber 2.0c curve fitting software. Binding dissociation equilibrium constants (K_{D}) and dissociative half-lives (t½) were then calculated from the kinetic rate constants as: K_{D}(M)=k_{d}/kₐ and t½ (min)=[In2/(60*k_{d})]. Binding kinetics parameters for different anti-PD-L1 monoclonal antibodies binding to different PD-L1 reagents at 25° C. and 37° C. are tabulated in Tables 3-8.

TABLE 3
Binding Kinetics parameters of anti-PD-L1
monoclonal antibodies binding to
hPD-L1-MMH at 25° C.
hPD-L1-MMH Monomer Binding at 25° C.

| | kₐ | k_{d} | K_{D} | t½ |
|---|---|---|---|---|
| Antibody | (1/Ms) | (1/s) | (M) | (min) |
| H2aM8306N | 1.44E+05 | 1.00E−02 | 6.96E−08 | 1.2 |
| H2aM8307N | 3.27E+04 | 1.47E−04 | 4.50E−09 | 79 |
| H2aM8309N | 6.42E+05 | 1.04E−02 | 1.63E−08 | 1.1 |
| H2aM8310N | 8.86E+04 | 3.10E−04 | 3.50E−09 | 37 |
| H2aM8312N | 7.59E+04 | 7.22E−04 | 9.51E−09 | 16 |
| H2aM8314N | 9.25E+05 | 5.17E−05 | 5.58E−11 | 224 |
| H2aM8316N | 9.57E+05 | 1.12E−03 | 1.17E−09 | 10 |
| H2aM8317N | 9.40E+04 | 3.95E−02 | 4.21E−07 | 0.3 |
| H2aM8321N | 1.03E+05 | 3.59E−03 | 3.49E−08 | 3.2 |
| H2aM8323N | 9.37E+05 | 2.23E−04 | 2.38E−10 | 52 |
| H2aM8718N | 9.27E+05 | 8.01E−05 | 8.63E−11 | 144 |
| H2aM8719N | 8.64E+04 | 2.26E−03 | 2.62E−08 | 5.1 |
| H1H9323P | 5.51E+04 | 1.88E−02 | 3.41E−07 | 0.6 |
| H1H9327P | 4.19E+05 | 2.59E−04 | 6.19E−10 | 45 |
| H1H9329P | 1.77E+06 | 1.34E−01 | 7.58E−08 | 0.1 |
| H1H9336P | 7.92E+05 | 3.90E−04 | 4.92E−10 | 30 |
| H1H9345P2 | 9.02E+04 | 1.49E−02 | 1.65E−07 | 0.8 |
| H1H9351P2 | 4.56E+05 | 8.96E−04 | 1.96E−09 | 13 |
| H1H9354P2 | 4.76E+04 | 3.83E−04 | 8.04E−09 | 30 |
| H1H9364P2 | 9.32E+05 | 2.99E−04 | 3.21E−10 | 39 |
| H1H9373P2 | 2.65E+06 | 2.91E−04 | 1.10E−10 | 40 |
| H1H9382P2 | 7.90E+04 | 4.79E−03 | 6.06E−08 | 2.4 |
| H1H9387P2 | 2.64E+06 | 5.82E−02 | 2.21E−08 | 0.2 |
| H1H9396P2 | 1.72E+05 | 2.18E−03 | 1.27E−08 | 5.3 |

TABLE 4
Binding Kinetics parameters of anti-PD-L1 monoclonal
antibodies binding to hPD-L1-MMH at 37° C.

| | hPD-L1-MMH Monomer Binding at 37° C. | | | |
|---|---|---|---|---|
| | kₐ | k_{d} | K_{D} | t_{1/2} |
| Antibody | (1/Ms) | (1/s) | (M) | (min) |
| H2aM8306N | 1.80E+05 | 3.10E−02 | 1.72E−07 | 0.4 |
| H2aM8307N | 5.35E+04 | 8.79E−04 | 1.64E−08 | 13 |
| H2aM8309N | 1.06E+06 | 3.14E−02 | 2.97E−08 | 0.4 |
| H2aM8310N | 1.32E+05 | 1.28E−03 | 9.70E−09 | 9.0 |
| H2aM8312N | 8.89E+04 | 4.03E−03 | 4.53E−08 | 2.9 |
| H2aM8314N | 1.07E+06 | 1.50E−04 | 1.40E−10 | 77 |
| H2aM8316N | 1.01E+06 | 5.30E−03 | 5.24E−09 | 2.2 |
| H2aM8317N | 9.03E+04 | 5.85E−02 | 6.47E−07 | 0.2 |
| H2aM8321N | 1.01E+05 | 9.29E−03 | 9.16E−08 | 1.2 |
| H2aM8323N | 1.38E+06 | 6.84E−04 | 4.97E−10 | 17 |
| H2aM8718N | 1.08E+06 | 1.55E−04 | 1.44E−10 | 74 |
| H2aM8719N | 1.50E+05 | 5.76E−03 | 3.84E−08 | 2.0 |
| H1H9323P | 1.21E+05 | 4.25E−02 | 3.52E−07 | 0.3 |
| H1H9327P | 5.21E+05 | 4.29E−04 | 8.24E−10 | 27 |
| H1H9329P | 2.82E+06 | 3.29E−01 | 1.17E−07 | 0.04 |
| H1H9336P | 1.07E+06 | 7.88E−04 | 7.33E−10 | 15 |
| H1H9345P2 | 1.72E+05 | 3.40E−02 | 1.97E−07 | 0.3 |
| H1H9351P2 | 6.82E+05 | 1.68E−03 | 2.47E−09 | 6.9 |
| H1H9354P2 | 7.39E+04 | 1.14E−03 | 1.54E−08 | 10 |
| H1H9364P2 | 1.35E+06 | 5.63E−04 | 4.17E−10 | 21 |
| H1H9373P2 | 3.09E+06 | 5.58E−04 | 1.80E−10 | 21 |
| H1H9382P2 | 9.97E+04 | 1.07E−02 | 1.07E−07 | 1.1 |
| H1H9387P2 | 3.49E+06 | 1.37E−01 | 3.91E−08 | 0.08 |
| H1H9396P2 | 3.44E+05 | 1.13E−02 | 3.30E−08 | 1.0 |

TABLE 5
Binding Kinetics parameters of anti-PD-L1 monoclonal
antibodies binding to human PD-L1 Dimer at 25° C.
Human PD-L1 Dimer Binding at 25° C.

| | kₐ | k_{d} | K_{D} | t_{1/2} |
|---|---|---|---|---|
| Antibody | (1/Ms) | (1/s) | (M) | (min) |
| H2aM8306N | 7.39E+05 | 3.09E−04 | 4.18E−10 | 37 |
| H2aM8307N | 1.89E+04 | 2.89E−05 | 1.54E−09 | 399 |
| H2aM8309N | 3.36E+06 | 1.44E−04 | 4.29E−11 | 80 |
| H2aM8310N | 3.13E+05 | 5.71E−05 | 1.83E−10 | 202 |
| H2aM8312N | 2.47E+05 | 1.02E−04 | 4.13E−10 | 113 |
| H2aM8314N | 3.16E+06 | 1.35E−05 | 4.26E−12 | 859 |
| H2aM8316N | 3.08E+06 | 1.44E−04 | 4.68E−11 | 80 |
| H2aM8317N | 3.59E+05 | 4.50E−04 | 1.25E−09 | 26 |
| H2aM8321N | 8.13E+05 | 2.87E−04 | 3.53E−10 | 40 |
| H2aM8323N | 2.91E+06 | 2.05E−05 | 7.04E−12 | 565 |
| H2aM8718N | 3.20E+06 | 1.62E−05 | 5.06E−12 | 713 |
| H2aM8719N | 3.42E+05 | 2.62E−04 | 7.67E−10 | 44 |
| H1H9323P | 2.24E+05 | 1.69E−04 | 7.54E−10 | 68 |
| H1H9327P | 4.66E+05 | 7.87E−05 | 1.69E−10 | 147 |
| H1H9329P | 2.97E+06 | 7.68E−04 | 2.59E−10 | 15 |
| H1H9336P | 1.38E+06 | 1.09E−04 | 7.86E−11 | 106 |
| H1H9345P2 | 5.00E+05 | 2.37E−04 | 4.74E−10 | 49 |
| H1H9351P2 | 9.16E+05 | 1.53E−04 | 1.67E−10 | 76 |
| H1H9354P2 | 1.68E+05 | 1.16E−04 | 6.89E−10 | 100 |
| H1H9364P2 | 2.42E+06 | 1.06E−04 | 4.37E−11 | 109 |
| H1H9373P2 | 4.08E+06 | 1.06E−04 | 2.60E−11 | 109 |
| H1H9382P2 | 2.23E+05 | 1.48E−04 | 6.63E−10 | 78 |
| H1H9387P2 | 5.07E+06 | 2.17E−04 | 4.27E−11 | 53 |
| H1H9396P2 | 7.76E+05 | 1.90E−04 | 2.45E−10 | 61 |

TABLE 6
Binding Kinetics parameters of anti-PD-L1 monoclonal
antibodies binding to human PD-L1 Dimer at 37° C.
Human PD-L1 Dimer Binding at 37° C.

| | kₐ | k_{d} | K_{D} | t_{1/2} |
|---|---|---|---|---|
| Antibody | (1/Ms) | (1/s) | (M) | (min) |
| H2aM8306N | 9.97E+05 | 4.16E−04 | 4.17E−10 | 28 |
| H2aM8307N | 4.41E+04 | 2.21E−04 | 5.00E−09 | 52 |
| H2aM8309N | 4.41E+06 | 1.66E−04 | 3.76E−11 | 70 |
| H2aM8310N | 4.81E+05 | 1.24E−04 | 2.58E−10 | 93 |
| H2aM8312N | 3.57E+05 | 2.61E−04 | 7.32E−10 | 44 |
| H2aM8314N | 3.89E+06 | 2.30E−05 | 5.91E−12 | 503 |
| H2aM8316N | 4.06E+06 | 2.37E−04 | 5.85E−11 | 49 |
| H2aM8317N | 4.81E+05 | 6.57E−04 | 1.36E−09 | 18 |
| H2aM8321N | 9.46E+05 | 2.69E−04 | 2.85E−10 | 43 |
| H2aM8323N | 4.32E+06 | 1.21E−04 | 2.80E−11 | 96 |
| H2aM8718N | 3.72E+06 | 1.98E−05 | 5.32E−12 | 584 |
| H2aM8719N | 4.37E+05 | 2.91E−04 | 6.66E−10 | 40 |
| H1H9323P | 5.19E+05 | 2.03E−04 | 3.91E−10 | 57 |
| H1H9327P | 6.83E+05 | 1.36E−04 | 2.00E−10 | 85 |
| H1H9329P | 3.87E+06 | 2.67E−03 | 6.89E−10 | 4.3 |
| H1H9336P | 2.75E+06 | 8.31E−05 | 3.02E−11 | 139 |
| H1H9345P2 | 6.82E+05 | 2.03E−04 | 2.97E−10 | 57 |
| H1H9351P2 | 1.25E+06 | 1.46E−04 | 1.17E−10 | 79 |
| H1H9354P2 | 4.56E+05 | 1.45E−04 | 3.17E−10 | 80 |
| H1H9364P2 | 3.34E+06 | 6.96E−05 | 2.08E−11 | 166 |
| H1H9373P2 | 5.12E+06 | 8.97E−05 | 1.75E−11 | 129 |
| H1H9382P2 | 4.92E+05 | 1.37E−04 | 2.78E−10 | 84 |
| H1H9387P2 | 6.12E+06 | 3.92E−04 | 6.39E−11 | 29 |
| H1H9396P2 | 1.09E+06 | 2.58E−04 | 2.37E−10 | 45 |

TABLE 7
Binding Kinetics parameters of anti-PD-L1 monoclonal
antibodies binding to MfPD-L1-MMH at 25° C.
MfPD-L1-MMH Monomer Binding at 25° C.

| | kₐ | k_{d} | K_{D} | t_{1/2} |
|---|---|---|---|---|
| Antibody | (1/Ms) | (1/s) | (M) | (min) |
| H2aM8306N | 1.65E+05 | 7.88E−03 | 4.77E−08 | 1.5 |
| H2aM8307N | 2.61E+04 | 1.07E−03 | 4.09E−08 | 11 |
| H2aM8309N | 8.70E+05 | 1.30E−02 | 1.49E−08 | 0.9 |
| H2aM8310N | 1.03E+05 | 3.06E−04 | 2.97E−09 | 38 |
| H2aM8312N | 7.09E+04 | 5.97E−04 | 8.42E−09 | 19 |
| H2aM8314N | 9.66E+05 | 7.00E−05 | 7.24E−11 | 165 |
| H2aM8316N | 9.71E+05 | 1.64E−03 | 1.69E−09 | 7.0 |
| H2aM8317N | 1.06E+05 | 2.38E−02 | 2.26E−07 | 0.5 |
| H2aM8321N | 1.34E+05 | 4.02E−03 | 2.99E−08 | 2.9 |
| H2aM8323N | 5.47E+05 | 8.68E−03 | 1.59E−08 | 1.3 |
| H2aM8718N | 9.04E+05 | 6.64E−05 | 7.35E−11 | 174 |
| H2aM8719N | 8.17E+04 | 2.68E−03 | 3.28E−08 | 4.3 |
| H1H9323P | 8.22E+04 | 2.40E−02 | 2.92E−07 | 0.5 |
| H1H9327P | 3.59E+05 | 3.33E−04 | 9.28E−10 | 35 |
| H1H9329P | 1.76E+06 | 1.35E−01 | 7.69E−08 | 0.09 |
| H1H9336P | 6.79E+05 | 5.94E−04 | 8.76E−10 | 19 |
| H1H9345P2 | 1.10E+05 | 8.50E−03 | 7.73E−08 | 1.4 |
| H1H9351P2 | 3.49E+05 | 1.11E−03 | 3.19E−09 | 10 |
| H1H9354P2 | 4.60E+04 | 3.05E−04 | 6.64E−09 | 38 |
| H1H9364P2 | 7.57E+05 | 3.12E−04 | 4.12E−10 | 37 |
| H1H9373P2 | 2.21E+06 | 2.82E−04 | 1.27E−10 | 41 |
| H1H9382P2 | 8.22E+04 | 1.29E−02 | 1.57E−07 | 0.9 |
| H1H9387P2 | 2.37E+06 | 6.04E−02 | 2.55E−08 | 0.2 |
| H1H9396P2 | 2.06E+05 | 2.52E−03 | 1.22E−08 | 4.6 |

TABLE 8
Binding Kinetics parameters of anti-PD-L1 monoclonal
antibodies binding to MfPD-L1-MMH at 37° C.
MfPD-L1-MMH Monomer Binding at 37° C.

| | kₐ | k_{d} | K_{D} | t_{1/2} |
|---|---|---|---|---|
| Antibody | (1/Ms) | (1/s) | (M) | (min) |
| H2aM8306N | 2.74E+05 | 3.68E−02 | 1.34E−07 | 0.3 |
| H2aM8307N | 1.87E+05 | 1.43E−03 | 7.63E−09 | 8.1 |
| H2aM8309N | 1.07E+06 | 2.81E−02 | 2.63E−08 | 0.4 |
| H2aM8310N | 4.71E+05 | 1.07E−03 | 2.27E−09 | 11 |
| H2aM8312N | 1.01E+05 | 3.32E−03 | 3.27E−08 | 3.5 |
| H2aM8314N | 1.07E+06 | 1.52E−04 | 1.42E−10 | 76 |
| H2aM8316N | 1.02E+06 | 5.38E−03 | 5.27E−09 | 2.1 |
| H2aM8317N | 2.66E+05 | 4.76E−02 | 1.79E−07 | 0.2 |
| H2aM8321N | 1.59E+05 | 8.16E−03 | 5.11E−08 | 1.4 |
| H2aM8323N | 9.56E+05 | 2.82E−02 | 2.95E−08 | 0.4 |
| H2aM8718N | 1.10E+06 | 1.46E−04 | 1.33E−10 | 79 |
| H2aM8719N | 1.35E+05 | 6.99E−03 | 5.19E−08 | 1.7 |
| H1H9323P | 1.25E+05 | 4.99E−02 | 3.98E−07 | 0.2 |
| H1H9327P | 4.77E+05 | 5.34E−04 | 1.12E−09 | 22 |
| H1H9329P | 2.66E+06 | 3.64E−01 | 1.37E−07 | 0.03 |
| H1H9336P | 9.09E+05 | 1.25E−03 | 1.38E−09 | 9.2 |
| H1H9345P2 | 1.64E+05 | 1.97E−02 | 1.21E−07 | 0.6 |
| H1H9351P2 | 5.60E+05 | 2.01E−03 | 3.59E−09 | 5.8 |
| H1H9354P2 | 8.59E+04 | 8.44E−04 | 9.82E−09 | 14 |
| H1H9364P2 | 1.12E+06 | 6.33E−04 | 5.66E−10 | 18 |
| H1H9373P2 | 2.81E+06 | 5.69E−04 | 2.03E−10 | 20 |
| H1H9382P2 | 1.30E+05 | 2.45E−02 | 1.89E−07 | 0.5 |
| H1H9387P2 | 3.20E+06 | 1.57E−01 | 4.89E−08 | 0.07 |
| H1H9396P2 | 3.94E+05 | 1.20E−02 | 3.05E−08 | 1.0 |

TABLE 9
Binding Kinetics parameters of anti-PD-L1 monoclonal
antibodies binding to monkey PD-L1-mFc at 25° C.
MfPD-L1-mFc Binding at 25° C.

| | kₐ | k_{d} | K_{D} | t_{1/2} |
|---|---|---|---|---|
| Antibody | (1/Ms) | (1/s) | (M) | (min) |
| H1H8314N | 2.58E+06 | 6.77E−05 | 2.63E−11 | 171 |
| H1H8316N | 2.71E+06 | 5.23E−05 | 1.93E−11 | 221 |
| H1H8323N | 2.67E+06 | 6.16E−05 | 2.31E−11 | 188 |
| H1H9351P2 | 7.81E+05 | 7.19E−05 | 9.22E−11 | 161 |
| H1H9364P2 | 1.32E+06 | 7.75E−05 | 5.87E−11 | 149 |
| H1H9373P2 | 2.85E+06 | 4.96E−05 | 1.74E−11 | 233 |
| H1H9387P2 | 3.55E+06 | 1.61E−04 | 4.52E−11 | 72 |
| H1H9351P2 | 8.64E+05 | 7.80E−05 | 9.03E−11 | 148 |
| H1H9364P2 | 1.25E+06 | 5.80E−05 | 4.62E−11 | 199 |
| H1H9373P2 | 3.27E+06 | 6.55E−05 | 2.00E−11 | 176 |
| H1H9387P2 | 2.90E+06 | 2.12E−04 | 7.30E−11 | 55 |
| H1H9323P | 2.80E+05 | 1.45E−04 | 5.19E−10 | 79 |
| H1H9327P | 3.48E+05 | 1.02E−04 | 2.94E−10 | 113 |
| H1H9329P | 1.74E+06 | 6.72E−04 | 3.86E−10 | 17 |
| H1H9336P | 9.90E+05 | 6.16E−05 | 6.22E−11 | 188 |
| H1H9344P2 | 3.50E+05 | 1.38E−04 | 3.93E−10 | 84 |
| H1H9345P2 | 2.96E+05 | 1.51E−04 | 5.12E−10 | 76 |
| H1H9354P2 | 2.05E+05 | 9.14E−05 | 4.47E−10 | 126 |
| H1H9382P2 | 2.13E+05 | 1.33E−04 | 6.26E−10 | 87 |
| H1H9396P2 | 8.37E+05 | 1.88E−04 | 2.25E−10 | 61 |

TABLE 10
Binding Kinetics parameters of anti-PD-L1 monoclonal
antibodies binding to monkey PD-L1-mFc at 37° C.
MfPD-L1-mFc Binding at 37° C.

| | kₐ | k_{d} | K_{D} | t_{1/2} |
|---|---|---|---|---|
| Antibody | (1/Ms) | (1/s) | (M) | (min) |
| H1H8314N | 2.89E+06 | 9.60E−05 | 3.33E−11 | 120 |
| H1H8316N | 2.96E+06 | 6.16E−05 | 2.08E−11 | 187 |
| H1H8323N | 2.99E+06 | 1.35E−04 | 4.51E−11 | 86 |
| H1H9351P2 | 1.06E+06 | 9.46E−05 | 8.91E−11 | 122 |
| H1H9364P2 | 2.36E+06 | 1.11E−04 | 4.71E−11 | 104 |
| H1H9373P2 | 3.15E+06 | 8.59E−05 | 2.73E−11 | 134 |
| H1H9387P2 | 3.41E+06 | 4.74E−04 | 1.39E−10 | 24 |
| H1H9351P2 | 1.61E+06 | 1.04E−04 | 6.47E−11 | 111 |
| H1H9364P2 | 2.41E+06 | 6.76E−05 | 2.80E−11 | 171 |
| H1H9373P2 | 3.86E+06 | 1.23E−04 | 3.19E−11 | 94 |
| H1H9387P2 | 2.90E+06 | 4.65E−04 | 1.61E−10 | 25 |
| H1H9323P | 3.84E+05 | 2.44E−04 | 6.36E−10 | 47 |
| H1H9327P | 7.64E+05 | 2.94E−04 | 3.85E−10 | 39 |
| H1H9329P | 2.18E+06 | 1.54E−03 | 7.08E−10 | 8 |
| H1H9336P | 1.86E+06 | 4.60E−05 | 2.47E−11 | 251 |
| H1H9344P2 | 9.05E+05 | 2.17E−04 | 2.40E−10 | 53 |
| H1H9345P2 | 8.61E+05 | 2.92E−04 | 3.39E−10 | 40 |
| H1H9354P2 | 2.72E+05 | 2.03E−04 | 7.46E−10 | 57 |
| H1H9382P2 | 2.84E+05 | 2.35E−04 | 8.25E−10 | 49 |
| H1H9396P2 | 1.57E+06 | 5.02E−04 | 3.19E−10 | 23 |

As shown in Table 3, at 25° C., all 25 anti-PD-L1 antibodies of the invention bound to hPD-L1-MMH with K_{D} values ranging from 55.8 pM to 421 nM. As shown in Table 4, at 37° C., all 25 anti-PD-L1 antibodies of the invention bound to hPD-L1-MMH with K_{D} values ranging from 140 pM to 647 nM. As shown in Table 5, at 25° C., all 25 anti-PD-L1 antibodies of the invention bound to hPD-L1 dimer with K_{D} values ranging from 4.26 pM to 1.54 nM. As shown in Table 6, at 37° C., all 25 anti-PD-L1 antibodies of the invention bound to hPD-L1 dimer with K_{D} values ranging from 5.32 pM to 5.0 nM. As shown in Table 7, at 25° C., all 25 anti-PD-L1 antibodies of the invention bound to MfPD-L1-MMH with K_{D} values ranging from 72.4 pM to 292 nM. As shown in Table 8, at 37° C., all 25 anti-PD-L1 antibodies of the invention bound to MfPD-L1-MMH with K_{D} values ranging from 133 pM to 398 nM. As shown in Table 9, at 25° C., all 20 anti-PD-L1 antibodies of the invention tested bound to MfPD-L1-mFc with K_{D} values ranging from 17.4 pM to 626 pM. As shown in Table 10, at 37° C., all 20 anti-PD-L1 antibodies of the invention tested bound to MfPD-L1-mFc with K_{D} values ranging from 20.8 pM to 825 pM.

### Example 4: Blocking of PD-L1 Binding to PD-1 as Determined by ELISA

The ability of monoclonal anti-PD-L1 antibodies to block human PD-L1 from binding to its binding partners, the human PD-1 and the human B7-1 receptors, was measured using two competition sandwich ELISA formats.

A dimeric human PD-1 protein comprised of a portion of the extracellular domain (amino acids 25-170 of accession number NP_005009.2 with a C93S change) that was expressed with a C-terminal hFc tag (hPD-1-hFc; SEQ ID: 350) and a dimeric human B7-1 protein comprised of a portion of the extracellular domain expressed with C-terminal hFc tag and hexahistidine tags (hB7-1-hFc-6His; R&D Systems, #140-B1) were separately coated at 2 μg/mL on a 96-well microtiter plate in a PBS buffer overnight at 4° C. Nonspecific binding sites were subsequently blocked using a 0.5% (w/v) solution of BSA in PBS. A constant amount of 0.5 nM or 8.0 nM of a dimeric hPD-L1 protein comprised of a portion of the human PD-L1 extracellular domain that was expressed with a C-terminal mFc tag (hPD-L1-mFc; SEQ ID: 348) was separately titrated with concentrations of anti-PD-1 antibodies and isotype control antibodies ranging between 0-210 nM in serial dilution. These antibody-protein complexes were then incubated for 1 hour at room temperature (RT). Complexes with 0.5 nM constant hPD-L1-mFc were subsequently transferred to microtiter plates coated with hPD-1-hFc, and complexes with 8 nM constant hPD-L1-mFc were transferred to hB7-1-hFc-6His coated plates. The complexes were allowed to bind to the coated plates for 1 hour at RT. After the 1 hour incubation, the wells were washed and plate-bound hPD-L1-mFc was detected with an anti-mFc polyclonal antibody conjugated with horse-radish peroxidase (Jackson ImmunoResearch, #115-035-164). Samples were developed with a TMB solution (BD Biosciences, #51-2606KC and #51-2607KC) to produce a colorimetric reaction and neutralized with 1M sulfuric acid before measuring absorbance at 450 nm on a Victor X5 plate reader. Data analysis used a sigmoidal dose-response model within Prism™M software. The calculated IC₅₀ value, defined as the concentration of antibody required to block 50% of hPD-L1-mFc binding to hPD-1-hFc or hB7-1-hFc-6His, was used as an indicator of blocking potency. Maximum blocking values represent the ability of the antibodies to block hPD-L1-mFc binding relative to baseline. The absorbance measured at the constant amount of hPD-L1 on the dose curve is defined as 0% blocking and the absorbance with no added hPD-L1 is defined as 100% blocking. The absorbance values of the wells containing the highest concentration for each antibody were used to determine the blocking percent at maximum concentration antibody tested. Antibodies with a maximum percent blockade below 25% were characterized as non-blockers, and their IC₅₀ values were not reported in Table 11. Antibodies with a maximum percent blockade below-25% were characterized as non-blockers/enhancers.

TABLE 11
ELISA blocking of hPD-L1-mFc binding to hPD-1-hFc and hB7-1-hFc-6His by anti-
PD-L1 antibodies

| | | | | | | Blocking 8 |
|---|---|---|---|---|---|---|
| | | | | | | nM of |
| | | | Blocking 0.5 | Highest | | hPD-L1- |
| | Highest | Blocking 0.5 | nM of | Antibody | Blocking 8 nM | mFc |
| | Antibody | nM of | hPD-L1-mFc | concentration | of | binding to |
| | concentration | hPD-L1-mFc | binding to | (nM) tested | hPD-L1-mFc | hB7-1-hFc- |
| | (nM) tested | binding to | hPD-1-hFc, | on hB7-1- | binding to | 6His, % |
| | on hPD-1- | hPD-1-hFc, | % maximum | hFc-6His | hB7-1-hFc- | maximum |
| Antibody | hFc coat | IC₅₀ (M) | blocking | coat | 6His, IC₅₀ (M) | blocking |
| H2aM8306N | 50 | <2.5E−10 (*) | 99 | 150 | <4.0E−09 (*) | 93 |
| H2aM8307N | 50 | IC | 29 | 150 | NBI/enhancer | −44 |
| H2aM8309N | 50 | <2.5E−10 (*) | 98 | 150 | <4.0E−09 (*) | 98 |
| H2aM8310N | 50 | 3.9E−10 | 76 | 150 | <4.0E−09 (*) | 98 |
| H2aM8312N | 50 | NBI | 23 | 150 | <4.0E−09 (*) | 98 |
| H2aM8314N | 50 | <2.5E−10 (*) | 93 | 150 | <4.0E−09 (*) | 97 |
| H2aM8316N | 50 | <2.5E−10 (*) | 96 | 150 | <4.0E−09 (*) | 98 |
| H2aM8317N | 50 | NBI | −11 | 150 | NBI/enhancer | −166 |
| H2aM8321N | 50 | NBI | 10 | 150 | NBI/enhancer | −124 |
| H2aM8323N | 50 | <2.5E−10 (*) | 100 | 150 | <4.0E−09 (*) | 99 |
| H2aM8718N | 50 | <2.5E−10 (*) | 97 | 150 | 1.0E−08 | 57 |
| H2aM8719N | 50 | 7.7E−10 | 95 | 150 | NBI/enhancer | −50 |
| H1H9323P | 50 | <2.5E−10 (*) | 43 | 210 | NBI/enhancer | −25 |
| H1H9327P | 50 | NBI/enhancer | −28 | 210 | IC | 41 |
| H1H9329P | 50 | <2.5E−10 (*) | 100 | 210 | 4.9E−09 | 100 |
| H1H9336P | 50 | <2.5E−10 (*) | 100 | 210 | 4.4E−09 | 99 |
| H1H9344P2 | 50 | NBI | 15 | 210 | NBI/enhancer | −51 |
| H1H9345P2 | 50 | <2.5E−10 (*) | 26 | 210 | NBI/enhancer | −34 |
| H1H9351P2 | 50 | <2.5E−10 (*) | 100 | 210 | 4.4E−09 | 100 |
| H1H9354P2 | 50 | 5.3E−10 | 34 | 210 | NBI | −13 |
| H1H9364P2 | 50 | <2.5E−10 (*) | 100 | 210 | 4.1E−09 | 101 |
| H1H9373P2 | 50 | <2.5E−10 (*) | 100 | 210 | 4.6E−09 | 101 |
| H1H9382P2 | 50 | <2.5E−10 (*) | 39 | 210 | NBI/enhancer | −30 |
| H1H9387P2 | 50 | <2.5E−10 (*) | 100 | 210 | <4.0E−09 (*) | 100 |
| H1H9396P2 | 50 | <2.5E−10 (*) | 59 | 210 | NBI | 8 |
| Isotype | 50 | NBI | −11 | 210 | NBI | −3 |
| control- | | | | | | |
| human IgG1 | | | | | | |
| Isotype | 50 | NBI | 5 | 210 | NBI | −3 |
| control- | | | | | | |
| mouse | | | | | | |
| IgG2a | | | | | | |

(*)-below theoretical bottom of the assay; Assay theoretical bottom is 2.5E−10 M for hPD-1-hFc coat and 4.0E−09 M for hB7-1-hFc-6His coat;
NBI—non-blocker;
IC—inconclusive

As shown in Table 11, 19 of the 25 anti-PD-L1 antibodies of the invention blocked 0.5 nM of hPD-L1-mFc from binding to hPD-1-hFc with IC₅₀ values ranging from less than 250 pM to 770 pM with maximum percent blockade ranging from 26% to 100%. Four of the 25 anti-PD-L1 antibodies tested were characterized as non-blockers of hPD-L1-mFc binding to hPD-1-hFc, while one antibody tested (H1H9327P) was characterized as a non-blocker/enhancer of hPD-L1-mFc binding to hPD-1-hFc. One antibody (H2aM8307N) demonstrated weak blocking of hPD-L1-mFc binding to hPD-1-hFc with a maximum percent blockade of 29%; however the IC₅₀ value could not be determined for this sample.

Further, 14 of the 25 anti-PD-L1 antibodies of the invention blocked 8nM of hPD-L1-mFc from binding to hB7-1-hFc-6His with IC₅₀ values ranging from <4 nM to 10 nM with maximum percent blockade ranging from 57% to 101%. Two of the 25 anti-PD-L1 antibodies tested were characterized as non-blockers of hPD-L1-mFc binding to hB7-1-hFc-6His, while 8 antibodies tested were characterized as non-blockers/enhancers of hPD-L1-mFc binding to hB7-1-hFc-6His. One antibody (H1H9327P) demonstrated weak blocking of hPD-L1-mFc binding to hB7-1-hFc-6His with a maximum percent blockade of 41%; however, the IC₅₀ value could not be determined for this sample.

### Example 5: Blocking of PD-L1 Binding to PD-1 as Determined by Biosensor Assay and by Surface Plasmon Resonance

Inhibition of human PD-L1 from binding to human PD-1 by different anti-PD-L1 monoclonal antibodies was studied either using real time bio-layer interferometry assay on an Octet Red96 biosensor instrument (Fortebio Inc.) or using a real-time surface plasmon resonance biosensor assay on a Biacore 3000 instrument.

Inhibition studies for anti-PD-L1 monoclonal antibodies expressed with mouse Fc were performed on an Octet Red96 instrument. First, 100 nM of a recombinant human PD-L1 expressed with a C-terminal mouse IgG2a Fc tag (hPD-L1-mFc; SEQ ID: 348) was incubated with 500 nM of each anti-PD-L1 monoclonal antibody for at least 1 hour before running the inhibition assay. Approximately 0.8 nm to 1.2 nm of recombinant human PD-1 expressed with a C-terminal human IgG1 Fc tag (hPD-1-hFc; SEQ ID: 350) was captured using anti-human IgG Fc capture biosensors. The Octet biosensors captured with hPD-1-hFc were subsequently submerged into wells containing the mixture of hPD-L1-mFc and different anti-PD-L1 monoclonal antibodies. The entire experiment was performed at 25° C. in Octet HBST buffer (0.01 M HEPES pH7.4, 0.15M NaCl, 3 mM EDTA, 0.05% v/v Surfactant P20, 0.1 mg/mL BSA) with a plate shaking at a speed of 1000 rpm. The biosensors were washed in Octet HBST buffer in between each step of the experiment. The real-time binding response was monitored during the course of the experiment and the binding response at the end of every step was recorded. Binding of hPD-L1-mFc to the captured hPD-1-hFc was compared in the presence and absence of different anti-PD-L1 monoclonal antibodies and was used to determine the blocking behavior of the tested antibodies as shown in Table 12.

TABLE 12
Inhibition of human PD-1 binding to human PD-L1 by different anti-PD-L1 monoclonal
antibodies expressed with mouse Fc performed on Octet Red96 instrument.

| | | Binding of the mixture of | |
|---|---|---|---|
| Anti-PD-L1 | Amount of | 100 nM hPD-L1-mFc and | |
| monoclonal | hPD-1-hFc | 500 nM anti-PD-L1 | % |
| antibody | Captured (nm) | monoclonal antibody (nm) | Blocking |
| No antibody | 1.05 | 0.31 | 0 |
| H2aM8306N | 0.98 | 0.02 | 94 |
| H2aM8307N | 1.01 | 0.46 | −48 |
| H2aM8309N | 0.89 | 0.02 | 94 |
| H2aM8310N | 0.95 | 0.13 | 58 |
| H2aM8312N | 1.06 | 0.52 | −68 |
| H2aM8314N | 0.99 | 0.02 | 94 |
| H2aM8316N | 1.06 | 0.01 | 97 |
| H2aM8317N | 0.92 | 0.58 | −87 |
| H2aM8321N | 1.04 | 0.60 | −94 |
| H2aM8323N | 1.00 | 0.02 | 94 |
| H2aM8718N | 1.08 | 0.01 | 97 |
| H2aM8719N | 0.93 | 0.11 | 65 |
| Isotype control antibody | 1.10 | 0.35 | −13 |

As shown in Table 12, 8 of the 12 anti-PD-L1 antibodies tested on the Octet Red96 instrument demonstrated blocking of hPD-L1-mFc from binding to hPD-1-hFc ranging from 58% to 97%. Four anti-PD-L1 antibodies tested demonstrated the ability to enhance the binding of hPD-L1-mFc to hPD-1-hFc.

Next, inhibition studies for anti-PD-L1 monoclonal antibodies expressed with human Fc were performed on Biacore 3000 instrument. First, 100nM of recombinant human PD-L1 expressed with a C-terminal human IgG1 Fc tag (hPD-L1-hFc; SEQ ID: 350) was incubated with 500nM of each anti-PD-L1 monoclonal antibody for at least 1 hour before running the inhibition assay. A CM5 Biacore sensor surface was first derivatized with anti-mouse IgG2a specific polyclonal antibody (Southern Biotech, #1080-01) using the standard EDC-NHS chemistry. Around 230 RUs of recombinant human PD-1 expressed with a C-terminal mouse IgG2a Fc tag (hPD-1-mFc; SEQ ID: 348) was then captured and was followed by an injection of 100nM of hPD-L1-hFc in the presence and absence of different anti-PD-L1 monoclonal antibodies at a flow rate of 25L/min for 2 minutes. The entire experiment was performed at 25° C. in HBST running buffer (0.01 M HEPES pH7.4, 0.15M NaCl, 3 mM EDTA, 0.05% v/v Surfactant P20).

The real-time binding responses were monitored during the entire course of the experiment and the binding response at the end of every step was recorded. Binding of hPD-L1-hFc to the captured hPD-1-mFc was compared in the presence and absence of different anti-PD-L1 monoclonal antibodies and was used to determine the blocking behavior of the tested antibodies as shown in Table 13.

TABLE 13
Inhibition of human PD-1 binding to human PD-L1 by different anti-PD-L1 monoclonal
antibodies expressed with human Fc performed on Biacore 3000 instrument.

| | | Binding of the mixture of | |
|---|---|---|---|
| Anti-PD-L1 | Amount of | 100 nM hPD-L1-hFc and | |
| monoclonal | hPD-1-mFc | 500 nM anti-PD-L1 | % |
| antibody | Captured (RU) | monoclonal antibody (nm) | Blocking |
| No mAb | 222 | 56 | 0 |
| H1H9323P | 224 | 262 | −368 |
| H1H9327P | 226 | 172 | −207 |
| H1H9329P | 227 | 2 | 97 |
| H1H9336P | 227 | 9 | 84 |
| H1H9345P2 | 229 | 292 | −422 |
| H1H9351P2 | 227 | 6 | 90 |
| H1H9354P2 | 229 | 296 | −428 |
| H1H9364P2 | 228 | 8 | 86 |
| H1H9373P2 | 227 | 6 | 89 |
| H1H9382P2 | 228 | 307 | −448 |
| H1H9387P2 | 228 | 5 | 91 |
| H1H9396P2 | 228 | 164 | −193 |
| Isotype control antibody | 228 | 56 | 0 |

As shown in Table 13, 6 out of 12 anti-PD-L1 antibodies of the invention tested on the Biacore 3000 instrument demonstrated blocking of hPD-L1-hFc from binding to hPD-1-mFc ranging from 84% to 97%. Six anti-PD-L1 antibodies tested demonstrated the ability to enhance the binding of hPD-L1-hFc to hPD-1-mFc.

Example 6: Octet cross-competition between anti-PD-L1 antibodies

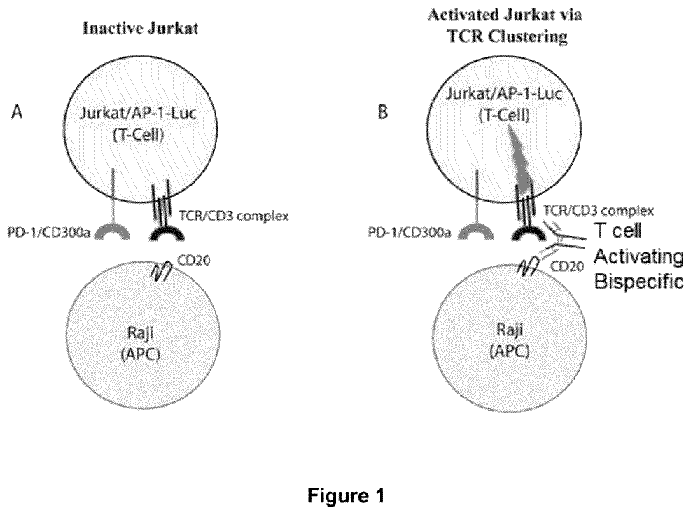

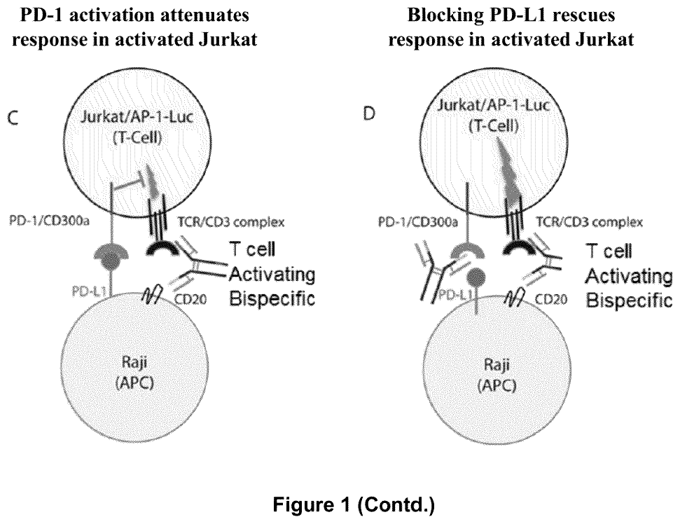

Binding competition between anti-PD-L1 monoclonal antibodies was determined using a real time, label-free bio-layer interferometry assay on an Octet RED384 biosensor (Pall ForteBio Corp.). The entire experiment was performed at 25° C. in Octet HBST buffer (0.01 M HEPES pH 7.4, 0.15M NaCl, 3 mM EDTA, 0.05% v/v Surfactant P20, 0.1 mg/mL BSA) with the plate shaking at the speed of 1000rpm. To assess whether 2 antibodies were able to compete with one another for binding to their respective epitopes on the recombinant human PD-L1 expressed with a C-terminal myc-myc-hexahistidine tag (hPD-L1-MMH; SEQ ID: 345), around ˜0.3 nm of hPD-L1-MMH was first captured onto anti-Penta-His antibody coated Octet biosensor tips (Fortebio Inc, #18-5079) by submerging the tips for 5 minutes into well containing 20 μg/mL solution of hPD-L1-MMH. The antigen captured biosensor tips were then saturated with first anti-PD-L1 monoclonal antibody (subsequently referred to as mAb-1) by dipping into wells containing 50 μg/mL solution of mAb-1 for 5 minutes. The biosensor tips were then subsequently dipped into wells containing 50 μg/mL solution of a second anti-PD-L1 monoclonal antibody (subsequently referred to as mAb-2). The biosensor tips were washed in Octet HBST buffer in between every step of the experiment. The real-time binding response was monitored during the course of the experiment and the binding response at the end of every step was recorded as shown in FIG. 1. The response of mAb-2 binding to hPD-L1-MMH pre-complexed with mAb-1 was compared and competitive/non-competitive behavior of different anti-PD-L1 monoclonal antibodies was determined.

Under the experimental conditions used in this Example, (a) H2aM8309N, H1H9329P, H1H9336P, H2aM8314N, H2aM8316N, H2aM8718N, H1H9387P2, H1H9351P2, H1H9364P2, H1H9373P2, and H2aM8306N cross-competed with each other; (b) H2aM8310N, H2aM8321N and H2aM8312N cross-competed with each other; (c) H1H9396P2, H2aM8317N, H2aM8321N, H1H9323P, H1H9382P2, H1H9344P2, H1H9345P2, and H1H9354P2 cross-competed with each other; and (d) H1H9327P and H2aM8307N cross-competed with each other. In one instance, competition was observed in one orientation but not in the opposite orientation: i.e., H2aM8307N when applied first competed with H2aM8309N, H1H9329P, H1H9336P, H2aM8314N, H2aM8316N, H2aM8718N, H1H9387P2, H1H9351P2, H1H9364P2, H1H9373P2, and H2aM8306N; however, in the opposite orientation, H2aM8309N, H1H9329P, H1H9336P, H2aM8314N, H2aM8316N, H2aM8718N, H1H9387P2, H1H9351P2, H1H9364P2, H1H9373P2, and H2aM8306N when applied first did not compete with H2aM8307N.

### Example 7: Antibody Binding to Cells Overexpressing PD-L1

The binding of anti-PD-L1 antibodies to a human embryonic kidney cell line (HEK293; ATCC, #CRL-1573) stably transfected with full length human PD-L1 (amino acids 1 to 290 of accession number NP_054862.1) (HEK293/hPD-L1) was determined by FACS.

For the assay, adherent cells were detached using enzyme-free dissociation buffer and blocked with complete medium. Cells were centrifuged and resuspended at a concentration of 2.8×10\hat{ }6 cells/mL in cold PBS containing 2% FBS. HEK293 parental and HEK293/hPD-L1 cells were then incubated for 15 to 30 minutes on ice with 100 nM of each anti-PD-L1 antibody or an isotype control antibody. Unbound antibodies were removed by washing with D-PBS containing 2% FBS, and cells were subsequently incubated with a phycoerythrin-conjugated secondary Fcγ fragment specifically recognizing either human Fc (Jackson ImmunoResearch, #109-116-170) or mouse Fc (Jackson ImmunoResearch, #115-115-164) for 15 to 30 minutes on ice. Cells were washed with D-PBS containing 2% FBS to remove unbound secondary detection reagents and fluorescence measurements were acquired using a HyperCyte (IntelliCyt, Inc.) flow cytometer. Data was analyzed using HyperCyte software.

TABLE 14
FACS binding of anti-PD-L1 antibodies to
HEK293/hPD-L1 cells and parental HEK293 cells

| | FACS on | FACS on | Ratio of |
|---|---|---|---|
| | HEK293 | HEK293/ | HEK293/hPD-L1 |
| | parental cells | hPD-L1 cells | to HEK293 |
| Antibody | [MFI] | [MFI] | parental cells |
| H1H9323P | 1909 | 154992 | 81 |
| H1H9327P | 2120 | 317592 | 150 |
| H1H9329P | 1504 | 282088 | 188 |
| H1H9336P | 2263 | 379009 | 168 |
| H1H9344P2 | 1691 | 200976 | 119 |
| H1H9345P2 | 1885 | 228406 | 121 |
| H1H9351P2 | 1685 | 289523 | 172 |
| H1H9354P2 | 2204 | 275839 | 125 |
| H1H9364P2 | 2066 | 323663 | 157 |
| H1H9373P2 | 2151 | 333236 | 155 |
| H1H9382P2 | 1473 | 205563 | 140 |
| H1H9387P2 | 1232 | 323793 | 263 |
| H1H9396P2 | 2340 | 227961 | 97 |
| H2aM8306N | 1286 | 316485 | 246 |
| H2aM8307N | 1382 | 73976 | 54 |
| H2aM8309N | 1160 | 192678 | 166 |
| H2aM8310N | 1357 | 14918 | 11 |
| H2aM8312N | 1380 | 158331 | 115 |
| H2aM8314N | 2053 | 194832 | 95 |
| H2aM8316N | 1601 | 172104 | 108 |
| H2aM8317N | 1270 | 67600 | 53 |
| H2aM8321N | 1322 | 112495 | 85 |
| H2aM8323N | 2250 | 163497 | 73 |
| H2aM8718N | 2225 | 194341 | 87 |
| H2aM8719N | 1272 | 133399 | 105 |
| mouse IgG | 1273 | 1115 | 0.9 |
| Isotype control | | | |
| human IgG1 | 1179 | 1099 | 0.9 |
| Isotype control | | | |
| human IgG4 | 1991 | 1839 | 0.9 |
| Isotype control | | | |

As shown in Table 14, all 25 anti-PD-L1 antibodies of the invention showed strong binding to the HEK293/hPD-L1 cells compared to binding on the parental HEK293 line.

### Example 8: Blocking of PD-L1-Induced T-Cell Down-Regulation in a T-Cell/APC Luciferase Reporter Assay

T-cell activation is achieved by stimulating T-cell receptors (TcR) that recognize specific peptides presented by major histocompatibility complex class I or Il proteins on antigen-presenting cells (APC). Activated TcRs in turn initiate a cascade of signaling events that can be monitored by reporter genes driven by transcription factors such as activator-protein 1 (AP-1), Nuclear Factor of Activated T-cells (NFAT) or Nuclear factor kappa-light-chain-enhancer of activated B cells (NFκb). T-cell response is modulated via engagement of co-receptors expressed either constitutively or inducibly on T-cells. One such receptor is programmed cell death protein 1 (PD-1), a negative regulator of T-cell activity. PD-1 interacts with its ligand, PD-L1, which is expressed on target cells including APCs or cancer cells, and this interaction results in the delivery of inhibitory signals by recruiting phosphatases to the TcR signalosome, resulting in the suppression of positive signaling.

A bioassay was developed to measure T cell signaling induced by interaction between APC and T cells by utilizing a mixed culture derived from two mammalian cell lines: Jurkat cells (an immortalized T cell line) and Raji cells (a B cell line) (FIG. 1). For the first component of the bioassay, Jurkat Clone E6-1 cells (ATCC, #TIB-152) were transduced with the Cignal Lenti AP-1 Luc Reporter (Qiagen-Sabiosciences, #CLS-011L) as per the manufacturer's instructions. The lentivirus encodes the firefly luciferase gene under the control of a minimal CMV promoter, tandem repeats of the TPA-inducible transcriptional response element (TRE) and a puromycin resistance gene. The engineered Jurkat cell line was subsequently transduced with a PD-1 chimera comprising the extracellular domain of human PD-1 (amino acids from 1 to 170 of human PD1; accession number NP_005009.2) and the trans-membrane and cytoplasmic domains of human CD300a (amino acids from 181 to 299 of human CD300a; accession number NP_009192.2). The resulting stable cell line (Jurkat/AP1-Luc/hPD1-hCD300a) was selected and maintained in RPMI/10% FBS/penicillin/streptomycin/glutamine supplemented with 500 ug/mL G418+1 ug/mL puromycin.

For the second component of the bioassay, Raji cells (ATCC, #CCL-86) were transduced with human PD-L1 gene (amino acids 1-290 of accession number NP_054862.1) that had been cloned into a lentiviral (pLEX) vector system (Thermo Scientific Biosystems, #OHS4735). Raji cells, positive for PD-L1 (Raji/hPD-L1) were isolated by FACS using a PD-L1 antibody and maintained in Iscove/10% FBS/penicillin/streptomycin/glutamine supplemented with 1 ug/mL puromycin.

To simulate the APC/T cell interaction, a bispecific antibody composed of one Fab arm that bindings to CD3 on T cells and the other one Fab arm binding that binds to CD20 on Raji cells (CD3xCD20 bispecific antibody; e.g., as disclosed in US20140088295) was utilized. The presence of the bispecific molecule in the assay results in the activation of the T cell and APC by bridging the CD3 subunits on T-cells to CD20 endogenously expressed on Raji cells. Ligation of CD3 with anti-CD3 antibodies has been demonstrated to lead to activation of T cells. In this bioassay, antibodies blocking the PD1/PD-L1 interaction rescue T-cell activity by disabling the inhibitory signaling and subsequently leading to increased AP1-Luc activation.

In the luciferase-based bioassay, RPMI 1640 supplemented with 10% FBS and penicillin/streptomycin/glutamine was used as assay medium to prepare cell suspensions and antibody dilutions to carry out the screening of anti-PD-L1 monoclonal antibodies (mAbs). On the day of the screening, EC₅₀ values of anti-PD-L1 mAbs, in the presence of a fixed concentration of CD3xCD20 bispecific antibody (30 pM), as well as the EC₅₀ of the bispecific antibody alone, were determined. In the following order, cells and reagents were added to 96 well white, flat-bottom plates. For the anti-PD-L1 mAb EC₅₀ determinations, first a fixed concentration of CD3xCD20 bispecific antibody (final 30 pM) was prepared and added to the microtiter plate wells. Twelve-point serial dilutions of anti-PD-L1 mAbs and controls were then added (final concentrations ranging from 1.7 pM to 100 nM; plus wells with assay medium alone). For the bispecific antibody (alone) EC₅₀ determination, the bispecific antibody, at final concentrations ranging from 0.17 pM to 10 nM (plus wells with assay medium alone), was added to the microtiter plate wells. Subsequently, a 2.5×10\hat{ }6/mL Raji/hPD-L1 cell suspension was prepared and 20 uL per well was added (final cell number/well 5×10\hat{ }4 cells). Plates were left at room temperature (15-20 minutes), while a suspension of 2.5×10\hat{ }6/mL of Jurkat/AP1-Luc/hPD1 (ecto)-hCD300a (TM-Cyto) was prepared. 20 uL of the Jurkat suspension (final cell number/well 5×10\hat{ }4 cells) was added per well. Plates containing the co-culture were incubated for 5 to 6 hours at 37° C. in 5% CO₂. Luciferase activity was then detected after the addition of ONE-Glo™ (Promega, #E6051) reagent and relative light units (RLUs) were measured on a Victor luminometer. All samples were tested in duplicates.

RLU values for each screened antibody were normalized by setting the assay condition with fixed (30 pM) concentration of the CD3/CD20 bispecific antibody, but without anti-PD-L1 antibody to 100%. This condition corresponds to the maximal AP1-Luc response elicited by the bispecific molecule in the presence of the PD-1/PD-L1 inhibitory signal. Upon addition of the anti-PD-L1 antibody, the inhibitory signal is suppressed, and the increased stimulation is shown here as Eₘₐₓ, the percentage increase in the signal in the presence of the highest antibody dose tested (100 nM). To compare potency of the anti-PD-L1 antibodies tested, the concentration of antibody at which the normalized RLU value reached 125% activation was determined from a four-parameter logistic equation over a 12-point response curve using GraphPad Prism. The results are summarized in Table 15.

TABLE 15
Anti-PD-L1 antibody blocking PD-1/PD-L1
dependent inhibition of AP1-Luc signaling

| | Antagonistic assay | Antagonistic assay |
|---|---|---|
| | Concentration [M] of | Emax mean |
| | Antibody at 125% | [%] @ |
| Antibody | activation | 100 nM |
| H2aM8306N | 3.78E−09 | 166.8 |
| H2aM8307N | N/A | 87.7 |
| H2aM8309N | 3.09E−10 | 180.9 |
| H2aM8310N | N/A | 112.8 |
| H2aM8312N | N/A | 75.6 |
| H2aM8314N | 1.44E−11 | 234.9 |
| H2aM8316N | 1.47E−10 | 177.4 |
| H2aM8317N | N/A | 109.2 |
| H2aM8321N | N/A | 116.8 |
| H2aM8323N | 2.20E−10 | 173.3 |
| H2aM8718N | 1.51E−10 | 182.0 |
| H1H9323P | N/A | 101.1 |
| H1H9327P | N/A | 77.1 |
| H1H9329P | N/A | 124.5 |
| H1H9336P | 9.81E−11 | 162.6 |
| H1H9344P2 | N/A | 98.3 |
| H1H9345P2 | N/A | 89.2 |
| H1H9351P2 | 3.44E−10 | 154.4 |
| H1H9354P2 | N/A | 89.9 |
| H1H9364P2 | 7.93E−11 | 164.5 |
| H1H9373P2 | 1.34E−10 | 150.2 |
| H1H9382P2 | N/A | 86.5 |
| H1H9387P2 | 1.86E−11 | 141.2 |
| H1H9396P2 | N/A | 102.6 |
| H1H8314N | 6.57E−11 | 147.9 |
| H1H9364P2 | 1.62E−10 | 158.1 |
| H1H9373P2 | 7.07E−11 | 152.5 |
| mlgG2a isotype control | N/A | 80.2 |
| hlgG1 isotype control | N/A | 96.8 |
| hlgG4 isotype control | N/A | 87.6 |

N/A = not applicable because at the concentrations tested these antibodies did not activate 125%

As shown in Table 15, 14 out of the 27 anti-PD-L1 antibodies of the invention tested blocked PD-1/PD-L1 inhibition with Eₘₐₓ values ranging from 234.9 to 138.1. Thirteen out of the 27 anti-PD-L1 antibodies of the invention did not demonstrate substantial blockade of PD1/PD-L1 interaction when tested in this assay. Isotype controls did not interfere with the PD1/PD-L1interaction.

### Example 9: In Vivo Efficacy of Anti-PD-L1 Antibodies

To determine the effect of a select number of anti-PD-L1 antibodies of the invention in a relevant in vivo model, an MC38.ova tumor growth study, involving subcutaneous injection of tumor cells and started on different days, was conducted in mice that were homozygous for the expression of the extracellular domain of human PD-L1 in place of extracellular domain of mouse PD-L1 (PD-L1 HumIn mice) on a 75% C57/BI6/25% 129 strain background. MC38.Ova (mouse colon adenocarcinoma) cells were engineered to express chicken ovalbumin in order to increase tumor immunogenicity, and to allow monitoring of the T-cell immune responses to well-defined antigenic ovalbumin peptides. In a second step, MC38.Ova cells were transduced with a lentiviral vector expressing hPD-L1 under SFFV promoter. MC38.Ova cells positive for hPD-L1 (MC38.Ova/hPD-L1) were isolated by FACS using hPD-L1-specific antibody. The cells were found to express low level of endogenous mouse PD-L1.

For a first study (study #1), mice were divided evenly according to body weight into 5 treatment or control groups (n=5 to 8 mice per group). At day 0, mice were anesthetized by isoflurane inhalation and then injected subcutaneously into the right flank with 1×10⁶ MC38.ova/hPD-L1 cells in suspension of 100 uL of DMEM. Treatment groups were intraperitoneally injected with 500 ug of either one of three anti-PD-L1 antibodies of the invention, or one of two isotype control antibodies with irrelevant specificity on days 3, 7, 10, 14, and 17 of the experiment, while one group of mice was left untreated.

In a second study (study #2), PD-L1 humanized mice were randomized into 7 treatment groups (n=5 to 6 mice). On day 0, mice were subcutaneously implanted with 1×10\hat{ }6 MC38.Ova/hPD-L1 cells. Mice were intraperitoneally administered with REGN a-PD-L1 ab (H1H8314N or H1H9364P2 or H1H9373P2), or isotype control abs hlgG4 mut or hlgG1 at doses of 10 mg/kg or 5 mg/kg. Groups of mice were administered antibody on days 3, 7, 10, 14, and 17. Tumor volumes were monitored by caliper measurement twice per week for the duration of the experiment (21 days). Experimental dosing and treatment protocol for groups of mice are shown in Table 16.

TABLE 16
Experimental dosing and treatment protocol for groups of mice

| | Samples | Dosage amount at each | | |
|---|---|---|---|---|
| Study # | tested | dosage time point | | Dosing interval |
| 1 | Isotype control 1 | 500 | μg | Days 3, 7, 10, 14, 17 |
| | H1H8314N | 500 | μg | Days 3, 7, 10, 14, 17 |
| | H1H9364P2 | 500 | μg | Days 3, 7, 10, 14, 17 |
| | H1H9373P2 | 500 | μg | Days 3, 7, 10, 14, 17 |
| | Isotype control 2 | 500 | μg | Days 3, 7, 10, 14, 17 |
| 2 | Isotype control 1 | 10 | mg/kg | Days 3, 7, 10, 14, 17 |
| | H1H8314N | 10 | mg/kg | Days 3, 7, 10, 14, 17 |
| | H1H8314N | 5 | mg/kg | Days 3, 7, 10, 14, 17 |
| | H1H9364P2 | 10 | mg/kg | Days 3, 7, 10, |
| | | | | 14, 17 |
| | H1H9364P2 | 5 | mg/kg | Days 3, 7, 10, |
| | | | | 14, 17 |
| | H1H9373P2 | 10 | mg/kg | Days 3, 7, 10, |
| | | | | 14, 17 |
| | H1H9373P2 | 5 | mg/kg | Days 3, 7, 10, |
| | | | | 14, 17 |

For the studies, average tumor volumes were monitored by caliper measurement twice per week for the duration of the experiment (17 days) and percent survival was recorded at the end of the experiment. In addition, the number of tumor-free mice was also assessed at the end of the study. Results, expressed as mean tumor volume (mm³) (±SD), percent survival, and number of tumor-free mice are shown in Tables 17 and 18.

TABLE 17
Mean tumor volume, percent survival and numbers of
tumor-free mice in each treatment group from study #1

| | Tumor Volume, mm³ | | | | Tumor-Free |
|---|---|---|---|---|---|
| | mean (±SD) | | Survival, % | | Mice |
| Antibody | Days 10 | Day 17 | Day 10 | Day 17 | Day 17 |
| Isotype Control 1 | 65 (±27) | 148 (±109) | 100% | 100% | 0/5 (0%) |
| Isotype Control 2 | 54 (±44) | 80 (±63) | 100% | 100% | 0/5 (0%) |
| H1H8314N | 6 (±10) | 2 (±5) | 100% | 100% | 4/5 (80%) |
| H1H9364P2 | 16 (±17) | 0 (±0) | 100% | 100% | 5/5 (100%) |
| H1H9373P2 | 13 (±14) | 0 (±0) | 100% | 100% | 5/5 (100%) |

As shown in Table 17 for study #1, all three anti-PD-L1 antibodies of the invention were efficacious in promoting tumor regression at the dosage of 500 ug/mouse with all mice from treatment groups that received two of the antibodies, H1H9364P2 and H1H9373P2, being tumor free at day 17. In the treatment group that received one of the anti-PD-L1 antibodies of the invention, H1H8314N, 4 out of 5 mice were tumor free by day 17, whereas 0 out of 5 animals were tumor-free in the isotype control groups. One-way ANOVA with Dunnett's multiple comparison post-test revealed a significant difference in tumor volumes between treatments with anti-PD-L1 antibodies of the invention and the isotype control antibody with a p value <0.05.

TABLE 18
Mean tumor volume, percent survival and numbers of tumor-free mice in each
treatment group from study #2

| | Tumor Volume, mm3 | | | | | | | | Tumor- | Tumor- |
|---|---|---|---|---|---|---|---|---|---|---|
| | Mean (SD) | | | | Survival, % | | | | free mice | free mice |
| | Day 10 | Day 10 | Day 21 | Day 21 | Day 10 | Day 10 | Day 21 | Day 21 | Day 21 | Day 21 |
| Antibody | 10 MPK | 5 MPK | 10 MPK | 5 MPK | 10 MPK | 5 MPK | 10 MPK | 5 MPK | 10 MPK | 5 MPK |
| Isotype | 55 (37) | N/A | 534 | N/A | 100% | N/A | 100% | N/A | 0/6 (0%) | N/A |
| control 1 | | | (356) | | | | | | | |
| H1H8314N | 14 (15) | 17 (4) | 19 | 108 | 100% | 100% | 100% | 100% | 3/6 | 2/5 (40%) |
| | | | 922) | (101) | | | | | (50%) | |
| H1H9364P2 | 18 (10) | 23 (10) | 34 (81) | 231 | 100% | 100% | 100% | 100% | 5/6 | 1/5 (20%) |
| | | | | (238) | | | | | (83%) | |
| H1H9373P2 | 10 (8) | 25 929) | 7 (16) | 37 (59) | 100% | 100% | 100% | 100% | 5/6 | 3/5 (60%) |
| | | | | | | | | | (83%) | |

As shown in Table 18, in study #2, administration of the selected anti-PD-L1 antibodies resulted in inhibition of tumor growth promoting tumor regression. All three anti-PD-L1 antibodies were efficacious at the 10 mg/kg dose and 5 mg/kg dose and promoted tumor regression in treated mice in a dose dependent manner throughout the course of the experiment, whereas 0 out of 5 animals were tumor-free in the control group. One-way ANOVA with Tukey's multiple comparison post-test revealed a significant difference in tumor volumes between treatments with the anti-PD-L1 antibodies and isotype control antibody with p value <0.05 or lower.

### Example 10: Anti-Tumor Effects of a Combination of an Anti-PD-L1 Antibody and a VEGF Antagonist in a Mouse Early-Treatment Tumor Model

An early-treatment tumor model was developed to test the efficacy of a combination of an anti-PD-L1 antibody and a VEGF antagonist. In this model, the combination therapy is administered shortly after tumor implantation. The experiment also used an anti-PD-1 antibody alone and in combination with the VEGF antagonist. The anti-PD-L1 antibody used in this experiment was an anti-PD-L1 monoclonal antibody with V_{H}/V_{L} sequences of antibody “YW243.55S70” according to US20100203056A1 (Genentech, Inc.), with mouse IgG2a and which was cross-reactive with mouse PD-L1. The VEGF antagonist used in this experiment was aflibercept (a VEGF receptor-based chimeric molecule, also known as “VEGF-trap” or “VEGFR1R2-FcΔC1 (a),” a full description of which is provided elsewhere herein). The anti-PD-1 antibody used in this experiment was anti-mouse PD-1 clone “RPMI-14” with rat IgG2b (Bio X Cell, West Lebanon, NH).

For this experimental model, 1.0×10⁶ Colon-26 tumor cells were implanted sub-cutaneously into BALB/c mice at Day 0. Starting on Day 3, prior to the establishment of measurable tumors, mice were treated with one of the mono-or combination therapies, or control combination, as set forth in Table 19.

TABLE 19
Experimental Dosing and Treatment Groups

| Treatment Group | First Agent | Second Agent |
|---|---|---|
| Control | IgG2a isotype | hFc control |
| Combination | control (250 μg, IP) | (250 μg, SC) |
| VEGF Trap | IgG2a isotype | Aflibercept |
| only | control (250 μg, IP) | (10 mg/kg, SC) |
| anti-PD-1 | anti-PD-1 mAb | hFc control |
| only | RPMI-14 (250 μg, IP) | (250 μg, SC) |
| anti-PD-L1 | anti-PD-L1 mAb | hFc control |
| only | (250 μg, IP) | (250 μg, SC) |
| VEGF Trap + | anti-PD-1 mAb | Aflibercept |
| anti-PD-1 | RPMI-14 (250 μg, IP) | (10 mg/kg, SC) |
| VEGF Trap + | anti-PD-L1 mAb | Aflibercept |
| anti-PD-L1 | (250 μg, IP) | (10 mg/kg, SC) |

The various therapies were administered at five different time points over a two week period (i.e., injections at Day 3, Day 6, Day 10, Day 13 and Day 19).

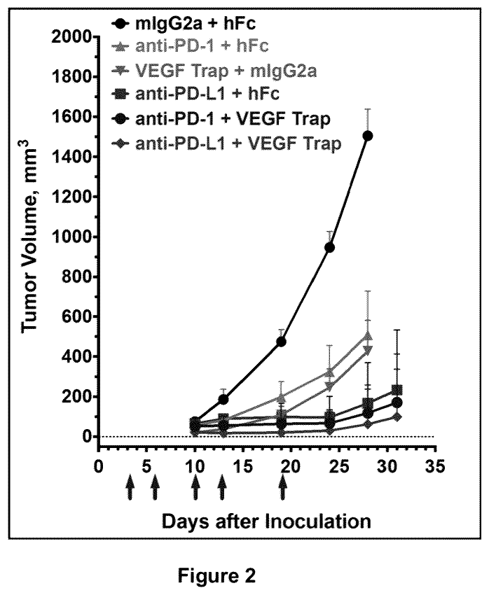

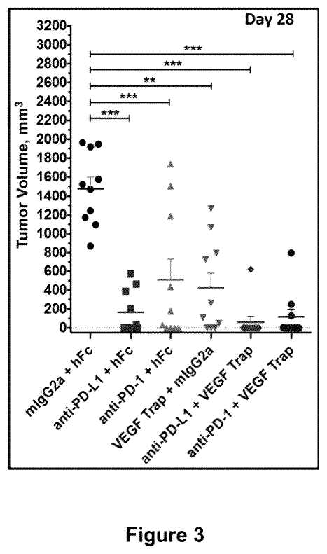

Animals in each therapy group were evaluated in terms of tumor incidence, tumor volume, median survival time, and number of tumor-free animals at Day 50. The extent of tumor growth is summarized in FIG. 2 (tumor growth curves) and FIG. 3 (tumor volume at Day 28). Results are also summarized in Table 20.

TABLE 20
Tumor-free mice upon treatment

| | No. of Tumor-Free |
|---|---|
| Treatment Group | Animals by Day 50 |
| Control Combination | 0/10 |
| VEGF Trap only | 3/10 |
| anti-PD-1 only | 4/10 |
| anti-PD-L1 only | 5/10 |
| VEGF Trap + anti-PD-1 | 7/10 |
| VEGF Trap + anti-PD-L1 | 9/10 |

Tumor growth was substantially reduced in animals treated with the combination of VEGF Trap+anti-PD-L1 antibody as compared with treatment regimens involving either therapeutic agent alone (see FIGS. 2 and 3). Furthermore, survival was substantially increased in the VEGF Trap+anti-PD-L1 antibody group, with 90% of animals surviving to at least day 50 after tumor implantation. By contrast, for the anti-PD-L1 and VEGF Trap monotherapy groups, survival to Day 50 was only 50% and 30% respectively (see FIG. 3 and Table 20).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Sequences having a length that is below the minimum length permitted under ST.26 format are provided in the following table:

| SEQ ID NO | Sequence |
|---|---|
| 13 | aaggcgtct |
| 14 | KAS |
| 29 | actgcatcc |
| 30 | TAS |
| 45 | gctgcatcc |
| 46 | AAS |
| 61 | aaggtttct |
| 62 | KVS |
| 77 | aagatttct |
| 78 | KIS |
| 93 | gttgcatcc |
| 94 | VAS |
| 109 | gttgcatcc |
| 110 | VAS |
| 125 | gctgcatcc |
| 126 | AAS |
| 141 | gctgcatcc |
| 142 | AAS |
| 157 | ggtgcatcc |
| 158 | GAS |
| 173 | gctgcatcc |
| 174 | AAS |
| 197 | gctgcatcc |
| 198 | AAS |
| 213 | gctgcatcc |
| 214 | AAS |
| 229 | gctgcatcc |
| 230 | AAS |
| 245 | gctgcatcc |
| 246 | AAS |
| 261 | gttgtgtcc |
| 262 | VVS |
| 277 | gctgcatcc |
| 278 | AAS |

1-42. (canceled)

43. A method of producing an isolated antibody or antigen-binding fragment thereof that binds specifically to programmed death-ligand 1 (PD-L1), comprising:
growing a host cell under conditions permitting production of the isolated antibody or antigen-binding fragment, and recovering the isolated antibody or antigen-binding fragment so produced,
wherein the host cell expresses a vector comprising: a first polynucleotide molecule, which comprises a first polynucleotide sequence that encodes three heavy chain complementarity determining regions (CDRs) (HCDR1, HCDR2, and HCDR3) of the isolated antibody or antigen-binding fragment thereof contained within a heavy chain variable region (HCVR) comprising an amino acid sequence of SEQ ID NO: 90; and a second polynucleotide molecule, which comprises a second polynucleotide sequence that encodes three light chain CDRs (LCDR1, LCDR2, and LCDR3) of the isolated antibody or antigen-binding fragment thereof contained within a light chain variable region (LCVR) comprising an amino acid sequence of SEQ ID NO: 82.

44. The method of claim 43, wherein HCDR1 comprises an amino acid sequence of SEQ ID NO: 84, HCDR2 comprises an amino acid sequence of SEQ ID NO: 86, HCDR3 comprises an amino acid sequence of SEQ ID NO: 88, LCDR1 comprises an amino acid sequence of SEQ ID NO: 92, LCDR2 comprises an amino acid sequence of SEQ ID NO: 94, and LCDR3 comprises an amino acid sequence of SEQ ID NO: 96.

45. The method of claim 43, wherein the HCVR comprises an amino acid sequence of SEQ ID NO: 82.

46. The method of claim 43, wherein the LCVR comprises an amino acid sequence of SEQ ID NO: 90.

47. The method of claim 43, wherein the HCVR comprises an amino acid sequence of SEQ ID NO: 82 and the LCVR comprises an amino acid sequence of SEQ ID NO: 90.

48. The method of claim 43, wherein the isolated antibody or antigen-binding fragment thereof comprises an IgG1 or IgG4 isotype.

49. The method of claim 43, wherein the isolated antibody or antigen-binding fragment thereof is a multi-specific antigen-binding molecule.

50. The method of claim 43, wherein the host cell is a CHO cell.

51. A method of producing a pharmaceutical composition comprising an isolated antibody or antigen-binding fragment thereof that binds specifically to programmed death-ligand 1 (PD-L1), comprising: producing the isolated antibody or antigen-binding fragment according to the method of claim 43; and formulating the isolated antibody or antigen-binding fragment thereof with a pharmaceutically acceptable carrier or diluent to produce the pharmaceutical composition.
